(19) United States
(12) Patent Application Publication
(10) Pub. No.: US 2005/0118603 A1
(43) Pub. Date: Jun. 2, 2005

(54) Target detection system having a conformationally sensitive probe comprising a nucleic acid based signal transducer

(75) Inventors: Keun Ho Chun, Seoul (KR); Hyun Jin Hwang, Seoul (KR)

Correspondence Address: EDWARDS & ANGELL, LLP, P.O. BOX 55874, BOSTON, MA, 02205, US

(73) Assignee: Ahram Biosystems Inc.

(21) Appl. No.: 10/684,346

(22) Filed: Oct. 10, 2003

Related U.S. Application Data
(63) Continuation-in-part of application No. 10/684,230, filed on Oct. 10, 2003, now abandoned.
(60) Provisional application No. 60/417,864, filed on Oct. 11, 2002.

(51) Int. Cl. C12Q 1/68; C07H 21/04

(52) U.S. Cl. 435/6; 536/24.3

(57) ABSTRACT

Disclosed is a system for detecting at least one target agent in a sample. The system generally includes at least one probe adapated to relate presence of the target agent to a detectable change in probe conformation. Preferred probes include a conformationally responsive signal transducer that reports association of the target agent and the probe by detectably shifting from one hybridization state to another. The invention has a wide spectrum of important applications including use in the rapid detection of target agents in biological, industrial, and environmental samples.

### CROSS-REFERENCE TO RELATED APPLICATION

The present application is a continuation-in-part of U.S. Ser. No. ______ as filed on Oct. 10, 2003 which application has the same title as the present application and which claims priority to provisional application Ser. No. 60/417,864 as filed on Oct. 11, 2002 by Chun, K. H and H. J. Hwang. The present application also claims priority to the 60/417,864 provisional application. The disclosures of the U.S. Ser. No. ______ and 60/417,864 applications are each incorporated by reference.

### FIELD OF THE INVENTION

The present invention generally relates to a system for detecting at least one target agent in a sample. The system includes a probe adapted to relate presence of the target agent to a change in probe conformation. Typical probes include a nucleic acid based signal transducer that reports association of the target agent and the probe by shifting from one hybridization state to another in a detectable manner. The invention has important applications including use for detecting a wide spectrum of target agents in a biological, pharmaceutical, industrial, or environmental sample.

### BACKGROUND OF THE INVENTION

The term “probe” has been used to refer to a composition that incorporates a target recognition element coupled to an appropriate signaling system. Certain probes can be used to detect chemical or biological target agents that specifically interact with the recognition element. Such probes are often designed to work reversibly and continuously to monitor the target agents in real time.

There have been efforts to develop a wide range of probes for use in the fields of chemistry, biology, medicine, pharmaceuticals, industrial and environmental monitoring, public safety and other areas (See e.g., Lakowicz, J. R. (1994); Leiner, M. J. P. and Hartmann, P. (1993); Aizawa, M. (1995)). For some probes, a large variety of biochemical and biological entities can be used as the recognition element. For instance, antigen-antibody interactions, enzyme-substrate interactions, and receptor-ligand interactions have all been suggested. There has been recognition that synthetic or semi-synthetic interactions may also serve as a basis for developing recognition elements (e.g., association of crown ethers and calixaren with other molecules or ions). See e.g., Shinbo, T. et al. U.S. Pat. No. 4,942,149 and Leiner, M. J. P. et al. U.S. Pat. No. 5,952,491.

There have been efforts to use some known recognition elements as a basis for developing probe-based target recognition approaches. Probes for use in such approaches can be divided into labeled and unlabeled probes.

There have been reports that unlabeled probes require use of complex detection strategies and often cumbersome probe immobilization steps. For example, surface plasmon resonance (SPR) (Liedberg, B. et al. (1995); Taremi, S. S. and Prosise, W. W. U.S. Pat. No. 5,981,167), evanescent-field technique (Sutherland, R. M. et al. (1984); Hirschfeld, T. E. U.S. Pat. No. 4,447,546), quartz crystal microbalance technique (Carter, R. M. et al. (1995); Ebersole, R. C. et al. U.S. Pat. No. 5,658,732.), and TOF mass spectrometry (Griffin, T. J. et al. (1999)) have all been reported. In these examples, complex instrumentation serves as a signal transducer to detect binding of a target agent to an unlabeled probe.

Other types of probes require detectable labels incorporated in the probe as a component. For instance, such probes can be designed to register interaction between the target agent and the recognition element in the probe. A widely used signaling technique called ELISA (Van Vunakis H. (1980); Hampar, B. et al. U.S. Pat. No. 4,764,459.) utilizes an enzyme-labeled antibody to generate an amplified signal in response to binding of a target antigen (or antibody) to an immobilized probe antibody (or antigen). Nucleic acid hybridization probes (Caskey, C. T. and Gibbs, R. A. L. U.S. Pat. No. 5,578,458; Chehab, F. F. U.S. Pat. No. 5,489,507; Lucas, J. N. et al. U.S. Pat. No. 5,616,465) utilize fluorescently labeled oligonucleotides to detect target nucleic acid sequences.

There have been efforts to make probes that signal interaction between a desired target agent and the probe recognition element via a detectable conformation change. For example, U.S. Pat. No. 5,925,517 discloses dual conformation nucleic acid hybridization probes having light-generating interactive label pairs for detection of target nucleic acid sequences. As disclosed, the label pairs are in close proximity with each other (“off” state) in the absence of the target nucleic acid sequence. However when the probe binds to the target nucleic acid sequence, the probe changes conformation in a way that separates the label pairs (“on” state).

Other probe systems have been reported including a ligand-conjugated quencher-fluorescent polymer complex system for detection of target receptors. See Chen, L. et al. (1999); and Whitten, D. G. et al. PCT Pub. No. WO 01/85997 A1.

Other probe types have been reported. See Comoglio, S. and Celada, F. (1976); Gonnelli, M. et al. (1981); and Henderson, D. R. U.S. Pat. No. 4,708,929.

However many of the prior probes and probe-based detection strategies have substantial drawbacks. For instance, most of the probes are formatted specifically to detect nucleic acid or related targets. Other targets of potential interest such as those including amino acid sequences (e.g., polypeptides, ligand receptors, antibodies and enzymes) and synthetic molecules (e.g., crown ethers, dioxins, etc.) are not easily detected using these approaches. Moreover, most dual conformation probes are limited to use of interacting label pairs i.e., labels that require reversible association with each other to generate a detectable signal. Unfortunately, such probes are not readily used with other label types, thus constraining probe design and detection choices. In addition, the unitary feature of many reported dual conformation probes limits detection strategies to those involving intramolecular conformational changes. That is, the probes are not designed to register intermolecular conformational changes between two or more independent probes. These and other shortcomings are believed to exclude detection of a wide variety of target agents in a reproducible, sensitive and cost effective manner.

### SUMMARY OF THE INVENTION

The present invention generally relates to a system for detecting at least one target agent in a sample. In one aspect, the system includes at least one probe adapted to relate presence of the target agent in the sample to a detectable change in probe conformation. The probe includes a nucleic acid based signal transducer that reports association of the target agent and the probe by detectably shifting from one hybridization state to another. The invention has a broad spectrum of important applications including use in detecting target agents in a variety of samples of biological, industrial, or environmental interest.

Typical probes in accord with the invention include a confornationally sensitive nucleic acid based signal transducer that includes as operably linked components: (1) one or two pairs of complementary nucleic acid sequences and typically but not exclusively, (2) at least one detectable label. In one invention embodiment and under assay conditions in the absence of a desired target agent, the signal transducer assumes an “off” hybridization state. Presence of the target agent in the sample however induces the signal transducer to conformationally shift to an “on” hybridization state. As will be discussed below, the nucleic acid based signal transducer is compatible with a range of detection strategies and is not limited to any prior labeling approach.

More particular probes of the invention further include, as a third operably linked component (3), at least one target agent recognition element which is preferably in association with at least one of the complementary nucleic acid sequences e.g., one or both pairs, preferably one pair thereof. Such a recognition element is suitably coupled, either directly or indirectly, to one or both pairs of the complementary nucleic acid sequences. A particular probe recognition element is suitably adapted to provide one or more interaction sites for at least one of the desired target agents. Such a recognition element is further positioned within or near the probe and particularly the nucleic acid based signal transducer to induce (or help induce) the transducer to shift conformation in the presence of the target agent associated with the recognition element.

Typically, interaction of the target agent with the recognition element facilitates a destabilizing force that is passed to the nucleic acid based signal transducer, thereby inducing the transducer to shift conformation from the “off” hybridization state (absence of the target agent) to the “on” hybridization state (presence of the target agent). The destabilizing force is more specifically referred to herein as a “nucleic acid hybridization destabilizing force” in embodiments in which the signal transducer includes (whole or in part) a nucleotide sequence. In some embodiments however, the interaction between the target agent and the recognition element is sufficient to reduce or eliminate a hybridization destabilizing force that is already a feature of the nucleic acid based signal transducer. In this instance, reduction or elimination of such force induces the transducer to shift conformation from the “off” hybridization state to the “on” state. Probes according to the invention are particularly well-suited to convert interaction of a wide variety of target agents into a readily detectable transducer signal e.g., those arising from steric hindrance, hydrophobic, hydrophilic or charge-charge repulsion arising directly or indirectly by association of the target agent and the probe. Interaction forces generated whole or in part by secondary molecules associating with the target agent, the probe, or both are also envisioned and are detectable by the present target detection system. Examples include, but are not limited to, those target agents that form specific interacting (or interaction) pairs including receptor-ligand, antibody-antigen, enzyme-substrate pairs and the like.

More specific recognition elements in accord with the invention are optionally separable from the probe, particularly from the nucleic acid based signal transducer. In one embodiment, such a recognition element includes at least one adaptor, preferably less than five adaptors and typically one or two of same, that are intended to release at least part of the recognition element from the probe including substantially all of the recognition element upon interaction of the target agent. Alternatively, or in addition, the adaptor can serve to join the probe to another probe or probe component, including at least part of another recognition element that is optionally bound to at least one of the target agents. This optionally separable feature of the recognition element provides advantages and significantly expands the utility of the present detection system.

For example, in embodiments in which the recognition element is not separable from the probe, interaction of the target agent thereto typically produces an interaction force (i.e., a nucleic acid hybridization destabilizing force) that is passed to the probe and particularly the nucleic acid based signal transducer. In this invention embodiment, the transducer shifts its conformation from an “off” to an “on” hybridization state upon interaction of the target agent. Such a probe will sometimes be referred to herein as an “affinity” probe.

However, in embodiments in which the recognition element of the probe includes at least one of the adaptors, the recognition element can be irreversibly or reversibly cleaved (separated) from the probe either with or without an associated target agent by interaction of the target agent. In embodiments in which the probe includes such a cleavable adaptor and at least one target agent associated thereto, separation of the recognition element from the probe facilitates a detectable shift in the nucleic acid signal transducer conformation from an “off” hybridization state to one that is “on”. Such probes will sometimes be referred to herein as “cleavage” reaction probes. In this invention embodiment, the target agent will sometimes be referred to as a “reaction inducing agent” or like phrase.

In still other embodiments, the probe adaptor is formatted to specifically bind to another molecule (sometimes referred to herein as a “reaction group”) that is preferably associated with what is referred to herein as a destabilizing agent. For instance, if a particular adaptor is intended to irreversibly or reversibly join the reaction group to the signal transducer by interaction of the target agent (sometimes referred to herein as a “reaction inducing agent”), the probe will be referred to as a “Type I” or “Type II” coupling probe. Association of the destabilizing agent to these probes induces a detectable conformational shift in the signal transducer. In another embodiment however, at least part of the probe adaptor is rendered less reactive or unreactive by the presence of the reaction inducing agent, thereby reducing or blocking joining of the reaction group. This type of probe is often referred to herein as a “Type II(-)” coupling probe. Basis for discriminating between these probe types is discussed in more detail below.

The invention is fully compatible with a variety of joining strategies including those involving what is sometimes referred to as “reaction-inducing agent(s)” which preferably induce (or catalyze) a desired joining reaction. It is noted that a particular reaction-inducing agent is usually a target agent of interest. In most cases, the target agent will not have a reaction group and thus will not usually be joined to the probe directly. However, use of a target agent that itself includes reaction groups (or can be made to include such groups) is within the scope of the present target detection system. More generally, interaction of a desired target agent with a reaction group in this embodiment is not limited to those target agents that have reaction groups i.e., the target agent can be joined to the probe directly or indirectly via the reaction group as needed.

It is an object of the present invention to provide probes that are flexible and can be used in a wide variety of detection strategies. Thus it is important to point out that the “off” and the “on” hybridization states of the nucleic acid based signal transducers are not intended to be associated with any particular probe structure or conformation. More specifically, when the nucleic acid based signal transducer is “off” (indicating absence of the target agent), the transducer can assume a probability distribution among different (typically two) conformations, preferably substantially in favor of one conformation. When the nucleic acid based signal transducer is “on” (indicating presence of the target agent), the transducer can conformationally shift to a detectably different probability distribution, preferably substantially in favor of the other conformation. Choice of whether to use a particular detection strategy will be guided by parameters understood by the user of this invention including the target agent to be detected, labeling approach, the particular recognition element selected, assay conditions employed and like considerations.

In embodiments in which the transducer can assume a substantially double stranded (duplex) or single stranded form depending on the presence or absence of the target agent, such transducers will be typically used in a non-competitive format described below. In addition, a probe that has an “off” hybridization state which is “substantially” in favor of one conformation and an “on” hybridization state which is “substantially” in favor of the other conformation will often be generally preferred.

As will be appreciated, detection of the target agent by the present detection system is closely tied to the ability of the nucleic acid based signal transducer to register and report to the user a detectable conformational shift in the presence or absence of the desired target agent. Moreover, it is not tied to any particular hybridization structure of the transducer either in the presence or absence of that target agent. However an important feature of the present detection system is that the probes do not detectably shift conformation in the absence of the intended target agent(s).

Probes according to the invention including the aforementioned affinity and reaction probes can be further characterized into non-competitive and competitive detection formats as follows.

In one embodiment of the non-competitive probe detection format, the nucleic acid based signal transducer includes (1) a pair of complementary nucleic acid sequences and typically but not exclusively, (2) at least one detectable label. The conformational shift in which the transducer signals presence of the target agent is registered by the probe transducer as a change in intermolecular hybridization between the pair of complementary nucleic acid sequences i.e., as a shift from a substantially single- (double) stranded conformation to a more double- (single) stranded conformation.

In another example of the non-competitive probe detection format, the nucleic acid based signal transducer is featured as a single strand that is preferably at least partially self-complimentary. Sometimes, such a probe will be referred to herein as a “unimolecular” probe. In this embodiment, the signal transducer is “off” in the absence of the target agent indicating substantial intramolecular hybridization (or de-hybridization in the case of the cleavage or Type II(-) cleavage probe) between the self-complimentary portions of the probe. However interaction between the target agent and the recognition element of the unimolecular probe facilitates an intramolecular conformational shift whereby transducer assumes an “on” hybridization state that is indicative of a presence of the target agent and, more specifically, to formation of a substantially single- (or double- in the case of the cleavage or Type II(-) coupling probe) stranded probe. By way of illustration, such a unimolecular probe is labeled with interactive labels i.e., label pairs that are matched such that at least one label group can alter at least one measurable characteristic of another label group when positioned in relatively close proximity in one conformation of the unimolecular probe but not when sufficiently separated in the other conformation. Examples of suitably interactive labels are discussed below.

As mentioned, conformationally sensitive probes intended for use in a competitive detection format are also envisioned.

For instance, and in one embodiment, the nucleic acid based signal transducer is formatted to include what is referred to herein as an “object” sequence and at least two complimentary sequences (sometimes referred to as “first” and “second” complement sequences). The first and second compliment sequences are typically designed to hybridize (either intermolecularly or intramolecularly) to different but overlapping portions of the object sequence. Such overlapping regions of complimentarily to the object sequence provide basis for thermodynamic competition between the first and second complement sequences to hybridize to the object sequence, particularly the overlapping sequence. Preferably, at least one recognition element is associated with the nucleic acid signal transducer so that interaction of the target agent thereto preferentially blocks (or enhances) hybridization of one of the compliment sequences to the object sequence (i.e., the first or the second complement sequence), while not preferentially blocking (or enhancing) hybridization of the other compliment sequence to the object sequence. Thus in one invention example, the nucleic acid based signal transducer reports presence of the target agent in association with the recognition element by shifting conformation sufficient to favor hybridization of the first (or second) compliment sequence to the object sequence while disfavoring hybridization of the second (or first) complement sequence to that object sequence. As discussed, it is preferred that hybridization involves the overlapping element of the object sequence. Accordingly, for most invention embodiments, the recognition element will not be closely associated with the overlapping sequence or otherwise interfere with the intended function of the signal transducer.

Accordingly, and in one aspect, the invention provides a target detection system that includes at least one probe as disclosed herein. In one embodiment, such a probe is particularly useful in an assay performed under conditions that include a detection temperature (or temperature range) that has been optimized to detect in a sample at least one target agent, preferably less then five of same, typically one or two target agents. A particular probe of interest includes at least one and preferably all of the following as operably linked components:
a) at least one nucleic acid based signal transducer comprising a first pair of nucleic acid sequences which pair includes a first object sequence and a first complement sequence in which the first object sequence and the first complement sequence are each independently between about 3 to about 150 nucleotides in length, the first object and first complement sequences being substantially complementary to each other so as to form an “off” hybridization state in the absence of the target agent and an “on” hybridization state in the presence of the target agent;
b) at least one recognition element operably linked to the nucleic acid signal transducer, wherein the recognition element specifically interacts with at least one target agent in the sample to be tested; and
c) optionally, at least one detectable label operably linked to the probe, preferably the nucleic acid signal transducer, wherein interaction of the target agent to the probe, particularly the recognition element conformationally shifts the “off” hybridization state to the “on” hybridization state, the detectable label producing a characteristic signal whose level is indicative of the amount of the “on” hybridization state provided by the probe transducer.

As discussed, the present detection system encompasses probes that can be used in a non-competitive or competitive detection format. In embodiments in which a competitive detection format is desired, such a probe will further include at least one pair of the following operably linked components:
d) a second object sequence embedded in the first pair of nucleic acid sequences and comprising between about 3 to about 150 nucleotides, preferably in association with the first object sequence to produce an overlapping region defined by the first and second object sequences and comprising less than about 40 nucleotides; and
e) a second complement sequence comprising between about 3 to about 150 nucleotides in length, the first and second complement sequences being essentially perfectly complementary to the overlapping region between the first and second object sequences, the second object sequence and the second complement sequence being substantially complimentary to each other over at least a length corresponding to the overlapping region sufficient to produce the “off” hybridization state in the absence of the target agent and the “on” hybridization state in the presence of the target agent.

The second object and second complement sequences are intended to facilitate the conformational shift from the “off” to the “on” state. When the target agent interacts with the nucleic acid based signal transducer of the probe, there is a conformational shift in the transducer from the “off” hybridization state intended to provide competitive binding contact between the overlapping region and the second or first complement sequence depending on the probe format. That shift produced by interaction of the target agent significantly favors thermodynamic exclusion of the first (or second) complement sequence from binding to the first (or second) object sequence and particularly the overlapping sequence. Thus in this invention embodiment, the “on” hybridization state is manifested by competitive binding of the second (or first) complement sequence and the substantial exclusion of the first (or second) complement sequence.

In most embodiments of the present detection system, the “on” hybridization state of the nucleic acid signal transducer is taken to be indicative of the presence of the target agent in the tested sample. Interaction of the target agent with the recognition element detectably alters (usually decreases) the amount of the “off” hybridization state of the nucleic acid signal transducer in favor of the “on” hybridization state. Such a conformational shift corresponds to an alteration (usually increases) in the characteristic signal produced by the detectable label (when present). In some invention embodiments such as those in which the target detection system uses certain affinity and reaction probes, the conformational shift embodied by the nucleic acid based signal transducer is thermodynamically reversible i.e., the transducer shifts conformation between the “off” and “on” hybridization state according to amount of the target agent present in the sample in a given time period.

In another aspect, the invention provides a detection system that includes at least one unimolecular probe as disclosed herein. In one embodiment, such a probe includes at least one and preferably all of the following as operably linked components:
a) at least one nucleic acid based signal transducer comprising a single-stranded nucleic acid sequence which sequence includes a first object sequence and a first complement sequence in which the first object and first complement sequences are each independently between about 3 to about 150 nucleotides in length, the first object and first complement sequences being substantially complementary to each other so as to form an “off” hybridization state in the absence of the target agent and an “on” hybridization state in the presence of the target agent;
b) at least one recognition element operably linked to the nucleic acid signal transducer, wherein the recognition element specifically interacts with at least one target agent; and
c) optionally, at least one detectable label operably linked to the probe, preferably the nucleic acid signal transducer, wherein interaction of the target agent to the probe, particularly the recognition element conformationally shifts the “off” hybridization state to the “on” hybridization state, the detectable label producing a characteristic signal whose level is indicative of the amount of the “on” hybridization state of the transducer.

In embodiments in which a competitive detection format is desired, such a probe will further include at least one pair of complementary nucleic acid sequences consisting of a second object sequence and a second complement sequence.

The invention also provides specific target detection systems in which one or more of the probes disclosed herein are operably linked to at least one, preferably one, two or three, solid supports. In one embodiment, such probes are covalently linked to a given solid support either directly or through one or more linkers preferably spaced between the solid support and the bound probe. Preferred solid supports are known in the field and include those intended for chromatographic, panning, biochip and related applications.

Also provided by the present invention are “pre-probes” and kits comprising those pre-probes. In one embodiment, the pre-probes include the following as operably linked (or operably linkable) components: a) at least one of the aforementioned nucleic acid based signal transducers and b) optionally, at least one of the adaptors. Such “pre-probes” can be readily converted into functional affinity or reaction probes of the invention, for example, by covalently linking one or more desired recognition elements to the nucleic acid based signal transducer with or without the adaptor. Preferred invention kits will include means for converting the pre-probes into desired affinity or reaction probes comprising a pre-determined target agent specificity.

Further provided by the invention are assays that use the detection system described herein. Preferred assays are adapted to detect a wide spectrum of different target agents including those specified below. Examples include, but are not limited to, assays for detecting chemicals including drugs (pharmaceuticals) and amino acid sequences e.g., antibodies, receptors, enzymes, cytokines, toxins, peptide hormones, growth factors, blood factors and the like including binding (or functional) fragments thereof. Also envisioned are assays for detecting lipids including phospholipids and carbohydrates including biologically important sugars such as glucose. For embodiments that cannot include a step or steps that separate labeled portions of the nucleic acid based signal transducer, use of certain of the unimolecular probes described herein will be preferred. Such methods can be performed in vitro, in situ or in vivo as needed. More preferred assays do not usually require extensive washing or separation steps to remove probes (or their components) not associated with target agent however such steps may improve performance of the detection system in some embodiments.

Assays suitable for use with the present detection system typically use at least one of the probes disclosed herein. In one embodiment, such use involves contacting the probe with a sample to be tested which sample has, is suspected of having, or will be made to have at least one target agent for which sensitive and reliable detection is needed. Such contact is conducted under conditions suited to detection format (e.g., the probe type used and the target agent to be detected) to determine if there is a detectable change in the hybridization state of the nucleic acid based signal transducer embedded in the probe when compared to a suitable control i.e., the measurable characteristic of the nucleic acid based signal transducer in the absence of the target agent. It will be apparent that in some embodiments it will be useful to run a control that includes no target agent (or no more than trace quantities thereof) and to compare the response of the sample to be tested to the response of the control. The level of signal produced by the nucleic acid based signal transducer may be measured quantitatively, semi-quantitatively or qualitatively as desired. For example, any detectable change registered by the signal transducer can be qualitatively assessed as being indicative of the presence of the target agent in the sample. It will be appreciated that in embodiments in which the present detection system is used in a pharmaceutical, clinical or diagnostic setting involving automated or semi-automated sample analysis, running the control may not always be necessary. In this instance, preliminary use of the detection system to produce a standard curve that relates presence of a particular amount of the target agent to a signal output (typically signal intensity) will be sufficient.

Accordingly, the invention also provides a method of using the present detection system to detect at least one target agent which is present in a sample to be tested, is suspected to be in that sample, or will be made to be included in that sample. In one embodiment, such a method includes contacting at least one of the probes disclosed herein with the sample under conditions sufficient to associate at least one of the target agents with the probe. Such contact is intended to facilitate a conformational shift in the nucleic acid based signal transducer from the “off” to the “on” hybridization state. Presence of the “on” hybridization state of the signal transducer is taken to be indicative of presence of one or more of the target agents. The transducer preferably outputs the signal arising from the “on” hybridization state to one or more detector implementations that are preferably formatted to retain the signal for future analysis, to report the output to the user in real-time, or both.

The invention also features assays for detecting a wide range of molecules that impact interaction between the target agent and the recognition element. Such molecules (“inhibitors”) will include those that reduce or eliminate the interaction. Alternatively, molecules that enhance the interaction between the target agent and recognition element (“enhancers”) are also fully detectable by the present methods.

One such a method involves adding to the present detection system one or more candidate compounds that are known, suspected to be, or that can be converted to an inhibitor (or enhancer) before, during or after addition to the system. Preferred addition to the target detection system is conductive to decreasing (or increasing) interaction between the target agent and the recognition element of a desired probe format. The method further includes detecting whether or not the selected probe format changes conformation to another hybridization state. Usually, but not exclusively, that change will be measured against a suitable control such as water, buffer and the like. Detection of the probe conformation change in the presence of the candidate compound is taken to be indicative of presence or absence of an inhibitor or enhancer in the target detection system.

It will be understood that the rate of conformational change of nearly any of the probes disclosed herein can be determined by applying standard analytical principles. The present invention is intended to encompass embodiments by which the methods detect presence or absence of desired target agents by detecting an increase or decrease in the rate of conformational change reported by the nucleic acid signal transducer.

Preferred probes according to the invention are sometimes referred to herein as “dual conformation” probes to denote the conformational switching between the “off” and “on” hybridization states of the nucleic acid based signal transducer. Such probes are advantageous in many aspects compared to other types of probes disclosed in the art.

For instance, because the signal transducer unit is incorporated within the probe as a molecular entity, extensive instrumentation or device except for conventional fluorometer or luminometer is not always necessary for generation and detection of the signal. Moreover, the dual conformation probes can be easily adapted to use in homogeneous assays which greatly simplify the detection scheme, for example compared to the methods such as ELISA or those using immobilized probes.

In the present invention, novel dual conformational probes which utilize nucleic acid hybrids conjugated to a variety of recognition elements are disclosed. The probes according to this invention are designed to undergo a conformational change of the nucleic acid hybrid(s), from a hybridized state to a dissociated state or vice versa, which sensitively responds to the interaction between the target agent and the recognition element conjugated to the probe. As the probes of the invention uses nucleic acid hybrids as a signal transducer unit, various recognized labeling methods can be used with the invention and are described below.

Particular probes according to the invention provide other advantages. For instance such probes can be readily adapted to use in detection of various chemical, biochemical, and biological target agents by changing the recognition element. For example, a ligand that can specifically bind to a receptor agent can be incorporated as a recognition element (sometimes more specifically referred to herein as a “probe ligand”) to construct an affinity probe to detect a target receptor agent or a target ligand. A substrate or a reaction site can be incorporated as a recognition element (sometimes more particularly referred to herein as a “probe substrate”) to detect reaction-inducing agents such as enzymes that catalyze cleavage reactions, coupling reactions, and modification reactions. Thus it will be apparent that what is meant by “recognition element” can often be guided by the probe context. As will be discussed in detail, the present invention provides various labeled dual conformation probes having selectivity and sensitivity needed for chemical and biochemical assays.

As discussed, the present target detection system is flexible and can be used in one or a combination of different label formats. As also discussed, most of the probes according to the invention are “optionally” labeled to point out that use of the target system is not always tied to presence of a detectably labeled invention probe.

For instance, and in one embodiment, the target detection system described herein is compatible with recognized signal amplification technologies such as those relying on the well-characterized beta-galactosidase reporter system. One such system reports use of enzyme donor and acceptor pairs as a basis for generating a detectable signal (See e.g., Comoglio, S. and Celada, F. (1976); Gonnelli, M. et al. (1981); Henderson, D. R. U.S. Pat. No. 4,708,929). The present target detection system can be readily adapted to conform to this technology e.g., by making invention probes that include as an interactive label pair the enzyme donor and the acceptor pair. In preferred use, the interactive label pair on the probe desirably amplifies the signal produced by the probe and specifically the nucleic acid based signal transducer.

Alternatively, or in addition, the target detection system of the present invention is compatible with use of standard polymerase chain reaction (PCR) (or polymerization reaction) amplification strategies. In one embodiment, nearly any of the invention probes described herein can be formatted to include one or more sequences that are made to serve as nucleic acid replication templates. More specifically, the forgoing object and complement sequences (first, second, or both) can be readily manipulated to include such sequence. Depending primarily on the conformationally sensitive hybridization state of the probe and particularly the nucleic acid based signal transducer, a polymerase or related enzyme can be used to facilitate a PCR reaction (or a polymerization reaction) and produce products stemming from the amplification. Presence of such amplification products or a detectable decrease in the amount of polymerization reagents (or both), can be taken to be indicative of the presence or absence of the target agent in the sample of interest.

Other advantages and features of the invention are described below.

### BRIEF DESCRIPTION OF THE DRAWINGS

FIG. 1 is a schematic representation of a non-competitive version of an affinity probe for detecting binding of a receptor agent in the hybridized (A) and dissociated (B) conformations, comprising a probe ligand and a pair of complementary nucleic acid sequences consisting of a first object sequence and a first complement sequence.

FIG. 2 is a schematic representation of a competitive version of an affinity probe for detecting binding of a receptor agent in the first (A) and second (B) hybridized conformations, comprising a probe ligand and two competing pairs of complementary nucleic acid sequences consisting of first and second object sequences and first and second complement sequences.

FIG. 3 is a schematic representation of a non-competitive version of a cleavage probe for detecting a cleavage reaction in the dissociated (A) and hybridized (B) conformations, comprising a probe substrate and a pair of complementary nucleic acid sequences consisting of a first object sequence and a first complement sequence.

FIG. 4 is a schematic representation of a competitive version of a cleavage probe for detecting a cleavage reaction in the second (A) and first (B) hybridized conformations, comprising a probe substrate and two competing pairs of complementary nucleic acid sequences consisting of first and second object sequences and first and second complement sequences.

FIG. 5 is a schematic representation of a non-competitive version of a Type I coupling probe for detecting a coupling reaction in the hybridized (A) and dissociated (B) conformations, comprising a probe substrate and a pair of complementary nucleic acid sequences consisting of a first object sequence and a first complement sequence.

FIG. 6 is a schematic representation of a non-competitive version of a Type II coupling probe for detecting a modification reaction in the hybridized (A and B) and dissociated (C) conformations, comprising a probe substrate and a pair of complementary nucleic acid sequences consisting of a first object sequence and a first complement sequence.

FIG. 7 is a schematic representation of a non-competitive version of a Type II(-) coupling probe for detecting a modification reaction in the hybridized (A and B) and dissociated (C) conformations, comprising a probe substrate and a pair of complementary nucleic acid sequences consisting of a first object sequence and a first complement sequence.

FIG. 8 is a schematic representation of a competitive version of a Type I coupling probe for detecting a coupling reaction in the first (A) and second (B) hybridized conformations, comprising a probe substrate and two competing pairs of complementary nucleic acid sequences consisting of first and second object sequences and first and second complement sequences.

FIG. 9 is a schematic representation of a competitive version of a Type II coupling probe for detecting a modification reaction in the first (A and B) and second (C) hybridized conformations, comprising a probe substrate and two competing pairs of complementary nucleic acid sequences consisting of first and second object sequences and first and second complement sequences.

FIG. 10 is a schematic representation of bimolecular probes having a non-interactive label in the hybridized conformations.

FIG. 11 is a schematic representation of immobilized bimolecular probes having a non-interactive label in the hybridized conformations.

FIG. 12 is a schematic representation of a trimolecular probe having two non-interactive labels in the first (A) and second (B) hybridized conformations.

FIG. 13 is a schematic representation of a trimolecular affinity probe used in Example 4 having a non-interactive label in the first (A) and second (B) hybridized conformations.

FIG. 14 is a schematic representation of immobilized trimolecular probes having at least one non-interactive label in the first hybridized conformations.

FIG. 15 is a schematic representation of an immobilized trimolecular affinity probe used in Example 9 having a non-interactive label in the first (A) and second (B) hybridized conformations.

FIG. 16 is a schematic representation of a bimolecular probe having a pair of interactive labels in the hybridized (A) and dissociated (B) conformations.

FIG. 17 is a schematic representation of a unimolecular probe having a pair of interactive labels in the hybridized (A) and dissociated (B) conformations.

FIG. 18 is a schematic representation of a unimolecular affinity probe used in Example 3 having a pair of interactive labels in the hybridized (A) and dissociated (B) conformations.

FIG. 19 is a schematic representation of a trimolecular probe having two pairs of interactive labels in the first (A) and second (B) hybridized conformations.

FIG. 20 is a schematic representation of a bimolecular probe having two pairs of interactive labels in the hybridized (A) and dissociated (B) conformations.

FIG. 21 is a schematic representation of a bimolecular probe having a pair of interactive labels in the hybridized (A) and dissociated (B) conformations.

FIG. 22 is a schematic representation of a trimolecular probe having two pairs of interactive labels in the first (A) and second (B) hybridized conformations.

FIG. 23 depicts the structures of the modified nucleotides used to construct the object and complement sequences used in the examples: a biotin-coupled thymidine (A), a fluorescein-coupled abasic nucleotide (B), a biotin-coupled abasic nucleotide (C), a DABCYL-coupled abasic nucleotide (D), a biotin-coupled abasic nucleotide (E), and a digoxigenin-coupled abasic nucleotide (F).

FIG. 24 depicts the structures of the modified nucleotides used to construct the kinase-specific probes used in Example 21: a carboxyl-coupled thymidine (A), a primary amine-coupled thymidine (B), a PKC recognition sequence-coupled thymidine (C), and a phosphorylated PKC recognition sequence-coupled thymidine (D).

FIG. 25 is a photograph of an acrylamide gel illustrating results of Example 1.

FIG. 26 is a photograph of an acrylamide gel illustrating results of Example 2.

FIG. 27 is a photograph of acrylamide gels illustrating results of Example 3.

FIG. 28 is a photograph of an acrylamide gel illustrating results of Example 4.

FIG. 29 is a photograph of acrylamide gels illustrating results of Example 5.

FIG. 30 is a photograph of an acrylamide gel illustrating results of Example 6.

FIG. 31 is a photograph of an acrylamide gel illustrating results of Example 7.

FIG. 32 is a photograph of an acrylamide gel illustrating results of Example 8.

FIG. 33 is a graph of fluorescence intensity illustrating results of Example 9.

FIG. 34 shows a photograph of microwells (A) and a graph of absorption from the microwells (B) illustrating results of Example 10.

FIG. 35A-F show temperature dependence of the fluorescence signal of the six unimolecular probes, UP3-12B (SEQ ID NO: 138), UP4-14B (SEQ ID NO: 139), UP5-16B (SEQ ID NO: 140), UP6-23B (SEQ ID NO: 141), UP7-33B (SEQ ID NO: 142), and UP8-43B (SEQ ID NO: 143), respectively, as a function of streptavidin concentration.

FIG. 36 shows relative increase of the fluorescence signals of the six unimolecular probes, UP3-12B (SEQ ID NO: 138), UP4-14B (SEQ ID NO: 139), UP5-16B (SEQ ID NO: 140), UP6-23B (SEQ ID NO: 141), UP7-33B (SEQ ID NO: 142), and UP8-43B (SEQ ID NO: 143), in the presence of ¼ molar equivalent of streptavidin relative to those measured in the absence of streptavidin at 25° C.

FIG. 37 shows the melting temperatures of the six unimolecular probes, UP3-12B (SEQ ID NO: 138), UP4-14B (SEQ ID NO: 139), UP5-16B (SEQ ID NO: 140), UP6-23B (SEQ ID NO: 141), UP7-33B (SEQ ID NO: 142), and UP8-43B (SEQ ID NO: 143), in the presence (solid circles) and absence (open circles) of streptavidin.

FIG. 38A-F show temperature dependence of the fluorescence signal of the six unimolecular probes, UP6-23B (SEQ ID NO: 141), UP9-23B (SEQ ID NO: 144), UP10-23B (SEQ ID NO: 145), UP11-23B (SEQ ID NO: 146), UP12-23B (SEQ ID NO: 147), and UP13-23B (SEQ ID NO: 148), respectively, as a function of streptavidin concentration.

FIG. 39 shows relative increase of the fluorescence signals of the six unimolecular probes, UP6-23B (SEQ ID NO: 141), UP9-23B (SEQ ID NO: 144), UP10-23B (SEQ ID NO: 145), UP11-23B (SEQ ID NO: 146), UP12-23B (SEQ ID NO: 147), and UP13-23B (SEQ ID NO: 148), in the presence of ¼ molar equivalent of streptavidin relative to those measured in the absence of streptavidin at 25° C.

FIG. 40 shows a fluorescence image of a microwell plate with each well containing assay solutions of the following compositions: 50 pmol of UP6-23B (SEQ ID NO: 141) in row A, B and C; 50 pmol of UP10-23B (SEQ ID NO: 145) in row D, E and F; 0, 6.25, and 12.5 pmol streptavidin in column 1, 2 and 3, respectively.

FIGS. 41A and B show temperature dependence of the fluorescence signal of the two unimolecular probes, UP1-34B (SEQ ID NO: 127) and UP14-21B (SEQ ID NO: 149), respectively, as a function of streptavidin concentration.

FIGS. 42A and B show temperature dependence of the fluorescence signal of the two unimolecular probes, UP15-23B (SEQ ID NO: 150) and UP16-23B (SEQ ID NO: 151), respectively, as a function of streptavidin concentration.

FIG. 43A-F show temperature dependence of the fluorescence signal of the six unimolecular probes, UP10-25B (SEQ ID NO: 152), UP10-23B (SEQ ID NO: 145), UP10-21B (SEQ ID NO: 153), UP10-19B (SEQ ID NO: 154), UP10-13B (SEQ ID NO: 155), and UP10-10B (SEQ ID NO: 156), respectively, as a function of streptavidin concentration.

FIG. 44A-D show temperature dependence of the fluorescence signal of the unimolecular probe UP6-23B (SEQ ID NO: 141) at different MgCl₂ concentration in the absence (circles) and presence (rectangles) of ¼ molar equivalent of streptavidin.

FIG. 45 shows temperature dependence of the fluorescence signal of the unimolecular probe UP6-23B (SEQ ID NO: 141) at different pH concentration in the absence (open marks) and presence (solid marks) of ¼ molar equivalent of streptavidin.

FIG. 46 shows inhibitor (biotin) concentration dependence of the fluorescence signal of the unimolecular probe UP6-23B (SEQ ID NO: 141) in the presence of ¼ molar equivalent of streptavidin at 25° C.

FIGS. 47A and B show temperature dependence of the fluorescence signal of two bimolecular probes, CM4* (SEQ ID NO: 157)+CM5-11B (SEQ ID NO: 158) and CM4* (SEQ ID NO: 157)+CM6-11B (SEQ ID NO: 159), respectively, as a function of streptavidin concentration.

FIG. 48 shows temperature dependence of the fluorescence signal of the bimolecular probe UP10-13B (SEQ ID NO: 155)+CM5 (SEQ ID NO: 160) as a function of streptavidin concentration.

FIG. 49A shows temperature dependence of the fluorescence signal of the two unimolecular probes, UP10-23PKCp (SEQ ID NO: 167) and UP10-23PKC (SEQ ID NO: 165) in the presence of ½ molar equivalent of anti-phosphothreinine antibody.

FIG. 49B shows inhibitor concentration dependence of the fluorescence signal of UP10-23PKC (SEQ ID NO: 165) after PKC enzyme reaction.

### DETAILED DESCRIPTION OF THE INVENTION

As discussed, the present invention provides a target detection system that includes one or a combination of conformationally sensitive probes as disclosed herein. Preferred probes according to the invention include at least one nucleic acid based signal transducer that is capable of reporting the presence or absence of one or more target agents. The target detection system is highly useful for detecting many naturally-occurring, synthetic (including recombinant) or semi-synthetic target agents including, but not limited to, receptor agents, ligands, environmental toxins, drugs, and reaction-inducing agents (e.g., a catalytic composition such as an enzyme). Also provided are a range of assays and kits for detecting the target agents using one or more probes (or sometimes pre-probes) according to the invention. The present target detection system is particularly useful in assays and especially screens that are designed to detect and optionally identify the target agent (or its inhibitors and enhancers).

By the phrase “target detection system” is meant at least one of the probes disclosed herein, preferably less than five of such probes, and typically one, two, or three of such probes, in combination with one or more other components that are intended to assist use. Such additional components will usually include a suitable aqueous medium for using the probes e.g., saline, buffer, water and the like. Such components can also include in some invention embodiments, a solid support that is operably linked to one or more of such probes. Examples of solid supports include those papers, resins, plastics, polymers, metals, metal oxides, glasses, silicon, silicon oxide, beads/spheres, dipsticks, wells, membranes and other solid supports known to be suitable for many chromatographic, panning, and/or biochip applications. Additionally contemplated components include stabilizers, additives, preservatives and the like that help to facilitate use of the target detection system. These include but are not limited to serum albumin, sodium azide, glycols and the like. If desired, the target detection system can be operably linked to (or combined with) a suitable detector(s) and related devices such as a computer-assisted implementation that is capable of collecting and reporting signal produced by the target detection system (as stored data or in real-time) and especially by the nucleic acid based signal transducer.

By the phrase “nucleic acid based signal transducer” or “signal transducer” including plural forms is meant a conformationally sensitive and responsive portion of a probe described herein that desirably responds to the presence (or absence) of one or more target agents. Particular transducers generally include nucleic acid sequences (e.g., DNA, RNA or combinations thereof), but may also include non-nucleotide sequences and particularly nucleotide analogues (e.g., thio-, aminoally-, and methyl-derivatives of adenosine, thymidine, guanosine, cytidine, and uradine mono-phosphates; inosine, queuosine, wybutosine, and pseudouridine mono-phosphates; and other derivatives thereof). Also envisioned are nucleic acid based signal transducers that include one or more radionucleotides e.g., ³H, ³²P, ³³P, ³⁵S, and ¹²⁵I. Signal transducers in accord with the invention can be single-stranded, double-stranded, or a combination thereof and have a length of typically less than about 250 base pairs, preferably less than about 150 base pairs, more preferably between about 3 to about 100 base pairs.

Most probes according to the invention include nucleic acid sequences that are usually made from DNA (deoxyribonucleic acid), RNA (ribonucleic acid), or mixtures of DNA and RNA. The nucleic acid may contain modified nucleotides including abasic nucleotides and nucleotides with additional chemical moieties linked thereto for the purpose of the invention. The links between the nucleotides may include bonds other than phosphodiester bonds, for example peptide bonds as in PNA (peptide nucleic acid). Modified internucleotide linkages are well known in the art and include methylphosphonates, phosphorothioates, phosphorodithionates, phosphoroamidites, and phosphate ester linkages. Dephospho-linkages are also known and include siloxane, carbonate, carboxylmethyl ester, acetamidate, carbamate, and thioether linkages. Plastic DNA, having for example N-vinyl, methacryloxyethyl, methacrylamide or ethyleneimine intemucleotide linkages can also be used. See e.g., Uhlmann and Peyman (1990). PNA is particularly useful because it is resistant to degradation by nucleases and also because it forms a stronger hybridized duplex with natural nucleic acids. See e.g., Orum et al. (1993) and Egholm, M. et al. (1993). See also U.S. Pat. No. 5,925,517 for related disclosure.

As discussed, preferred nucleic acid based signal transducers are conformationally sensitive and responsive to the presence, absence, or in certain embodiments, a change in amount of at least one target agent e.g., one, two or three of same. For instance, probe transducers disclosed herein are designed to be capable of detectably shifting conformation from an “off” hydridization state to one that is “on”. However, most of the nucleic acid based transducers are “reversible” in that they can also be used in detection strategies that require the transducer to detectably shift conformation from an “on” hybridization state to one that is “off”.

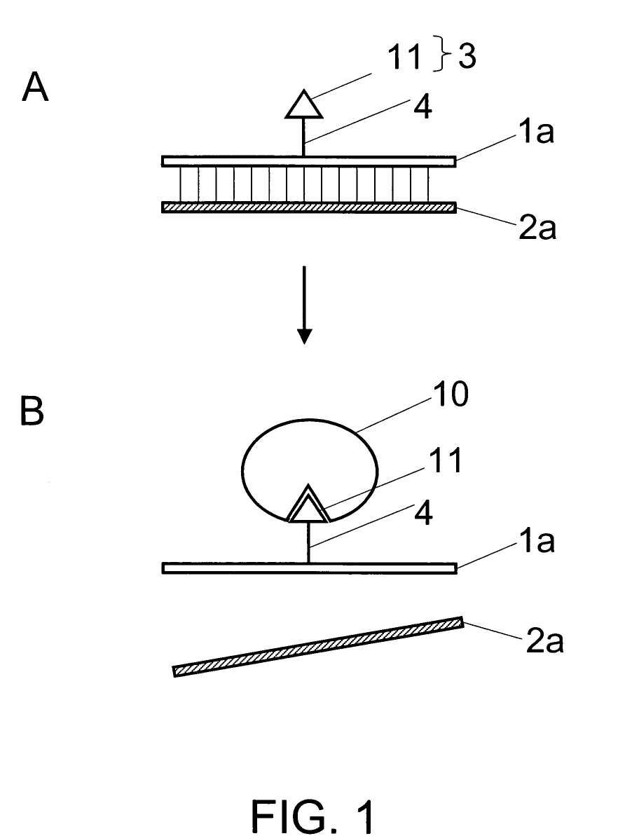

FIG. 1-22 illustrate a variety of particular probes that include specific nucleic acid based signal transducers. The transducers are shown detectably shifting conformation from “off” hybridization states to those that are “on” in the presence of a target agent.

As also discussed, the invention provides for a probe that includes at least one and preferably all of the following as operably linked components:
a) at least one nucleic acid based signal transducer (preferably less than five of same, and typically one or two) comprising a first pair of nucleic acid sequences which pair includes a first object sequence and a first complement sequence in which the first object sequence and the first complement sequence are each independently between about 3 to about 150 nucleotides in length, preferably about 4 to about 100 nucleotides in length, more preferably between from about 4 to about 50 nucleotides in length, the first object and first complement sequences being substantially complementary (preferably over at least 80%, 90% or greater of their respective lengths) to each other so as to form an “off” hybridization state in the absence of the target agent and an “on” hybridization state in the presence of the target agent;
b) at least one recognition element operably linked to the nucleic acid signal transducer, wherein the recognition element specifically interacts with at least one target agent in the sample to be tested; and
c) optionally, at least one detectable label operably linked to the probe, preferably the nucleic acid signal transducer, wherein interaction of the target agent to the probe, particularly the recognition element conformationally shifts the “off” hybridization state to the “on” hybridization state, the detectable label producing a characteristic signal whose level is indicative of the amount of the “on” hybridization state provided by the probe transducer.

By the phrase “operably linked”, “operably associated” or like phrase is meant one or more components of the target detection system that are operationally (i.e., functionally) linked to one or more other components. More specifically, and in embodiments in which one or more of the components of the target detection system include covalent linkage(s), such an association can be through carbon-carbon, carbon-oxygen bonding, phosphodiester bonding, peptide bonding, etc. However in other embodiments one or more of the components can be non-covalently linked by hydrogen bonding, salt bridges, ionic attractions and the like. Target detection systems in which some components are covalently linked and others non-covalently linked are within the scope of this invention.

It will be apparent that in embodiments in which one or more target detection system components are operably linked, character of that linkage can be impacted by factors such as conformational state and probe format. For example, in an invention example in which a pair of complementary nucleic acid sequences are non-covalently linked, for instance hybridized by hydrogen bonding in a hybridized conformation, those sequences can be spaced or separated from each other in a dissociated conformation. In another example of the invention in which one or more intercalating dyes are employed as a detectable label, the label will be non-covalently linked to (associated with) a hybridized conformation (duplex), but, in this illustration, not to a dissociated conformation. In still another embodiment, a destabilizing agent can be coupled to the Type I, II or II(-) “coupling” probe as a part of the probe, particularly as a part of the recognition element. In this example, the destabilizing agent will be initially separated from the probe, but covalently or non-covalently linked to the probe after the desired coupling reaction. Similarly, the destabilizing agent initially linked to the “cleavage” probe will be separated from the probe by interaction of the target reaction-inducing agent.

As used herein, the term “complementary sequence” or related phrases such as “complementary probe” is defined as the subunit sequence, usually comprising DNA or RNA, hybridizes with specific complementary to a nucleic acid sequence or subsequence. Percent complementary can be readily determined by routine experimentation or preferably by use of a suitable computer software program such as BLAST and related programs. Disclosure relating to using the BLAST program can be found at the United States National Center for Biotechnology Information (NCBI). By the phrase “substantially complementary” is meant at least 80%, preferably 90% or greater complementary.

As discussed, preferred probes of the invention include at least one recognition element. By the phrase “recognition element” is meant a probe component that includes, in one embodiment, at least one coupling element operably linked to at least one probe ligand or probe substrate. Typically, the coupling element will be a linking group whose size and number of atoms can change depending on intended use. Although it will generally be preferred to include a coupling element to space the probe ligand or probe substrate from the signal transducer, for some applications the coupling element may not be necessary.

More specifically, a “recognition element” according to the invention can include the probe ligand in embodiments in which an affinity probe is used. However, when a cleavage probe is employed, the recognition element will often include the cleavage site(s) and the destabilizing agent preferably connected by linker. In the Type I, II and II(-) coupling probes, the recognition element will typically include the reaction site. Alternatively, a preferred recognition element for use with Type I, II and II(-) coupling probes includes the reaction site, the destabilizing agent, and the reaction group. Examples of particular recognition elements are provided in the Drawings. Illustrations of particular coupling elements such as linkers are also disclosed herein.

Particular probes of the invention can be categorized into classes, for instance affinity probes for detecting receptor agents or ligands, and reaction probes for detecting reaction-inducing agents. The reaction probes are further categorized into four types depending on the nature of the reaction induced by the reaction-inducing agent. The first type is a cleavage probe for detecting a cleavage reaction in which a substrate is cleaved into at least two fragments by the action of the reaction-inducing agent. The second is a Type I coupling probe for detecting a coupling reaction in which at least two substrates are covalently linked by the action of the reaction-inducing agent. The third is a Type II coupling probe for detecting a modification reaction in which a reaction site in a substrate is converted to a conjugation site by the action of the reaction inducing agent. The last is a Type II(-) coupling probe for detecting a modification reaction in which a reaction site that is already a conjugation site is converted to a non-conjugatable site by the action of the reaction inducing agent. In the Type II and II(-) coupling probes, a second substrate is conjugated to the conjugation site either by a spontaneous binding or by a coupling reaction that takes place spontaneously or by the aid of a coupling reagent.

As used herein, the term “probe” is defined as any oligomer, comprising two or more nucleotide containing subunits (DNA, RNA, PNA), suitable for hybridization in accord with the invention. Such a probe may be labeled with a detectable moiety or may be unlabeled. Further, the probe may be in solution or immobilized to a solid support or solid carrier. More particular probe examples include DNA, RNA, DNA-RNA hybrid molecules, as well as oligomer that include peptide nucleic acids.

Additionally probes within the scope of the present invention include at least one bi- or tri-cyclic nucleoside analog as described in PCT WO 99/14226 to Wengel. Preferably, such probes include substitution with at least one “locked nucleic acid” (LNA) monomer unit. The manufacture and use of LNA and related analogs has been described. See also U.S. Pat. No. 6,268,490. Probes that include LNA and one or more of DNA, RNA and PNA are contemplated.

Further probes include at least one subunit that is a mimetic of a component of RNA and/or DNA. Examples include probes with certain oligonucleotide backbone modifications, sugar group modifications, and base modifications as described in U.S. Pat. No. 6,077,709 and references disclosed therein. Examples of such probes include those with phosphorothioate backbones, oligonucleotides with substituted sugar moieties such as 2′-methoxyribose, and modified nucleobases such as 5-methylcytosine. Conjugated probes are encompassed by the present invention i.e., those in which one probe has been chemically cross-linked with another moiety to another to enhance activity.

Most probes according to the invention include conformationally sensitive and responsive nucleic acid based signal transducers. Certain of such probes can be characterized as “dual-conformation” probes that undergo a conformational change depending on the presence or absence of the target agent. One form of conformational change involves hybridization and dissociation of one or two pairs of substantially complementary nucleic acid sequences induced by interaction between at least one recognition element included in the probe and the target agent(s). The probes having one or two pairs of the nucleic acid sequences will be referred as “non-competitive” or “competitive” probes, respectively, in the view of the non-competitive or competitive nature of the hybridization process.

In embodiments in which a competitive format is desirable, such probes will further include at least one pair of the following operably linked components (referring to components (a)-(c) above):
d) a second object sequence embedded in the first pair of nucleic acid sequences and comprising between about 3 to about 150 nucleotides in length, preferably about 4 to about 100 nucleotides in length, more preferably between from about 4 to about 50 nucleotides in length, preferably in association with the first object sequence to produce an overlapping region defined by the first and second object sequences and comprising less than about 40 nucleotides, preferably between from about 1 to about 30 nucleotides, more preferably about 3 to about 20 nucleotides; and
e) a second complement sequence comprising between about 3 to about 150 nucleotides in length, preferably about 4 to about 100 nucleotides in length, more preferably between from about 4 to about 50 nucleotides in length, the first and second complement sequences being essentially perfectly complementary to the overlapping region between the first and second object sequences, the second object sequence and the second complement sequence being substantially complimentary to each other over at least a length corresponding to the overlapping region sufficient to produce the “off” hybridization state in the absence of the target agent and the “on” hybridization state in the presence of the target agent.

As discussed, the second object and second complement sequences are intended to facilitate the conformational shift from the “off” to the “on” state.

As discussed, the invention probes include, as a third operably linked component, at least one target agent recognition element which is preferably in association with at least one of the object and complement nucleic acid sequences. In an embodiment in which the probe is competitive, it will include both pairs of the nucleic acid sequences. Preferably, the recognition element is conjugated to the first pair (but not the second pair) of the nucleic acid sequences, i.e., preferably to the first object sequence excluding the overlapping region or the first complement sequence.

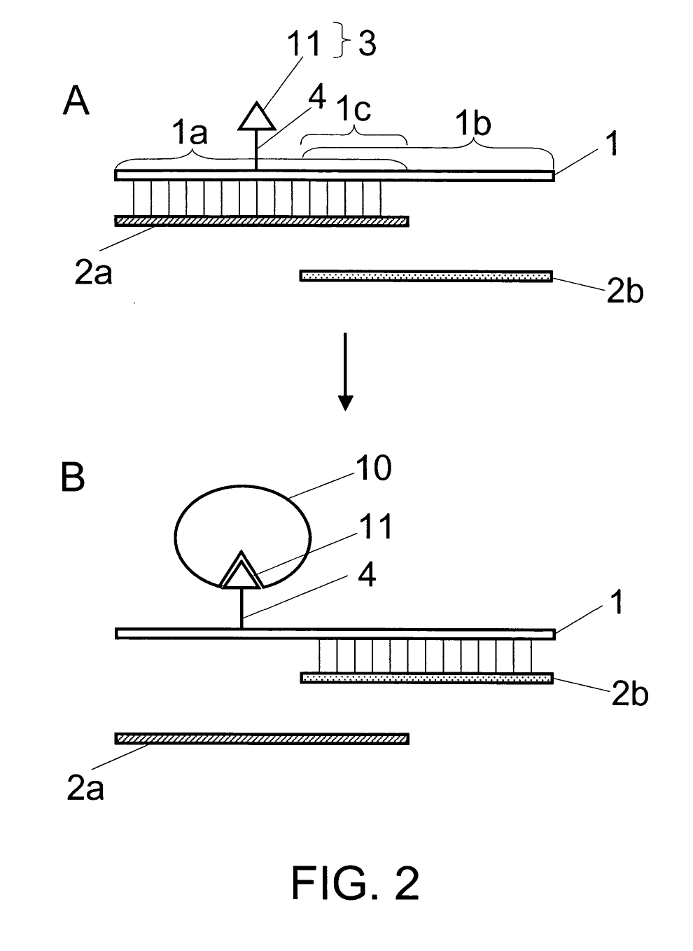

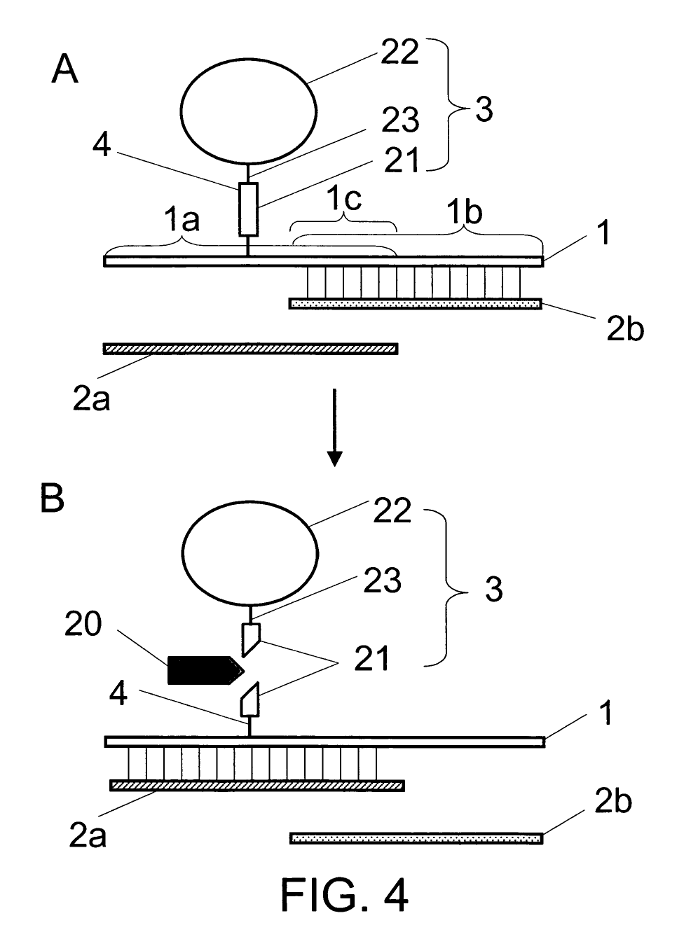

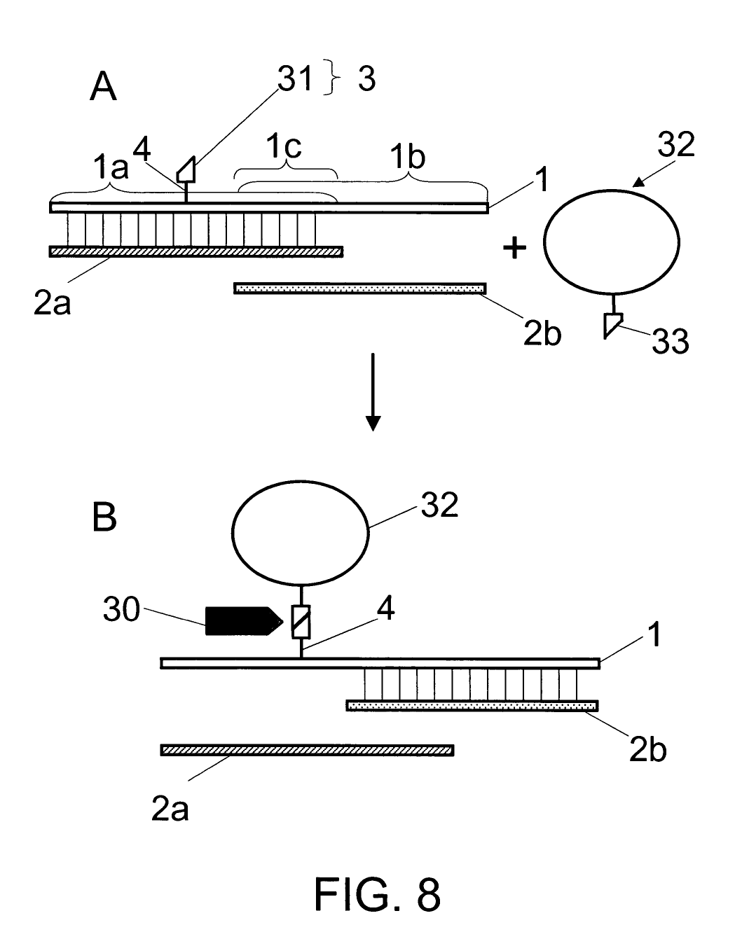

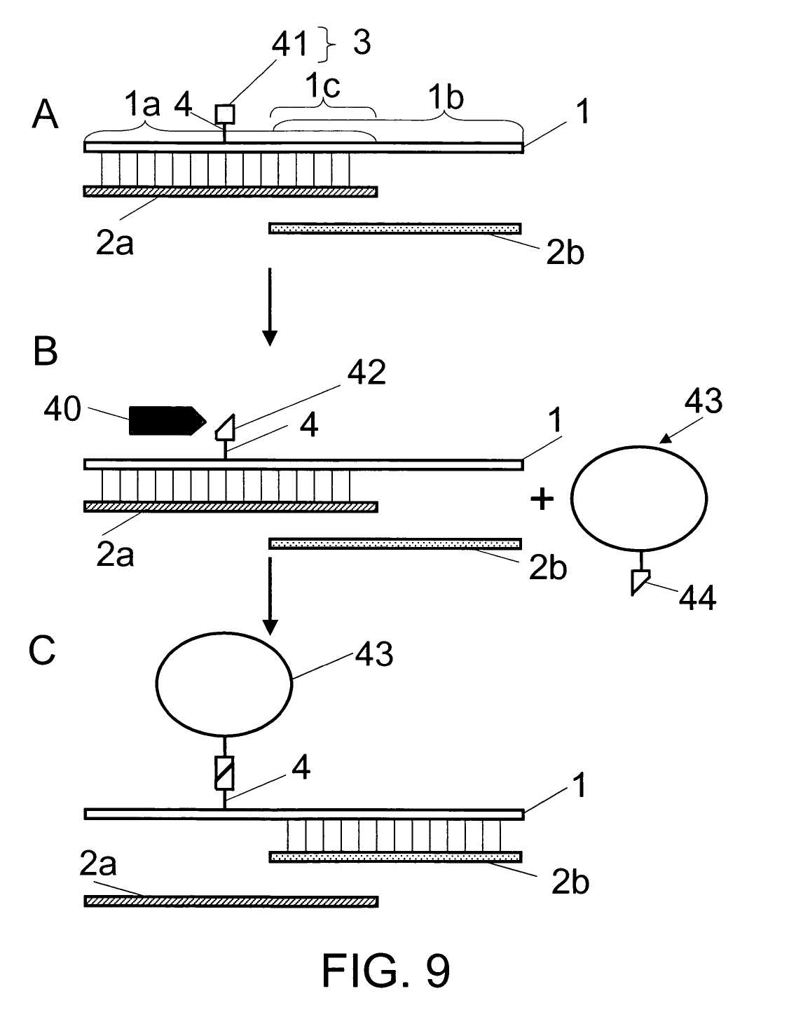

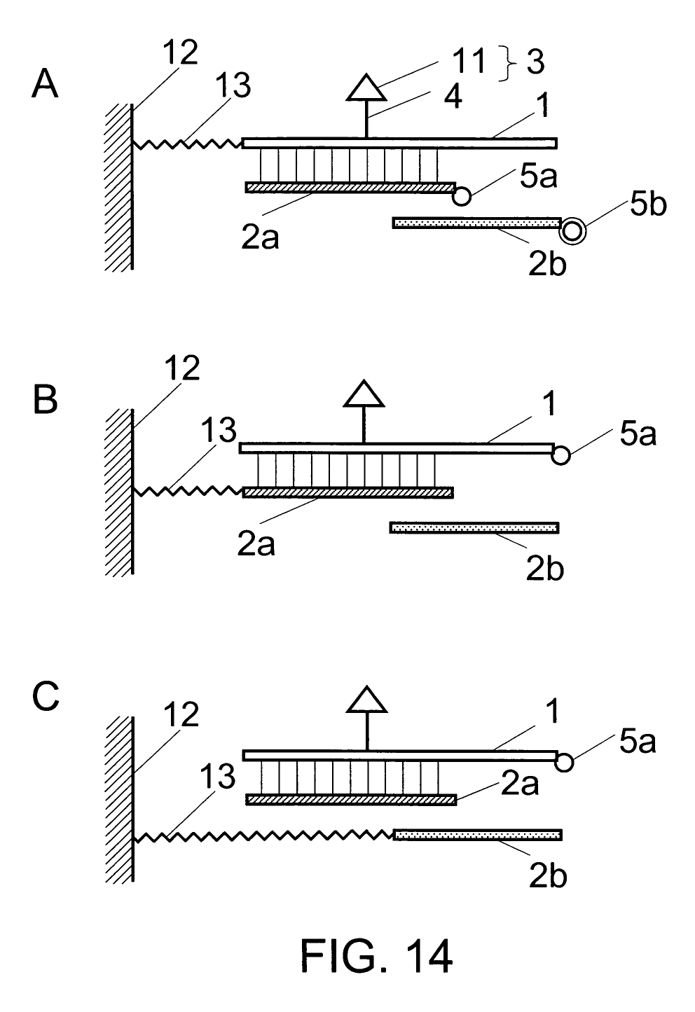

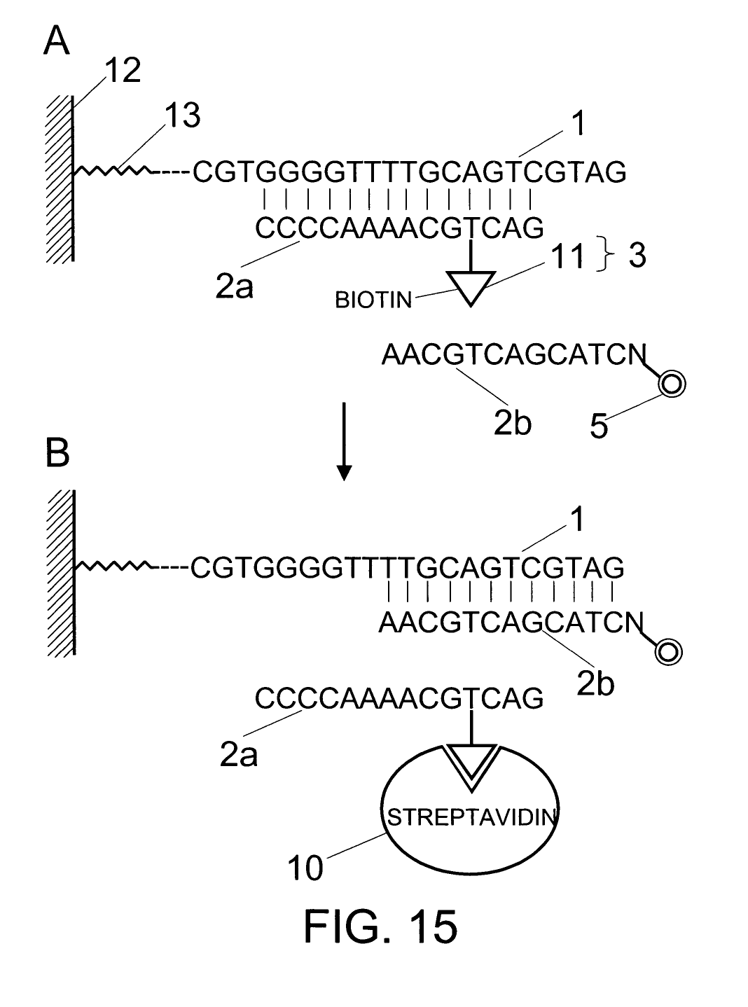

Illustrations of particular competitive probe formats are provided in FIGS. 2, 12, 13, 19, and 22 (affinity probe format showing different labeling strategies), FIG. 4 (cleavage), FIG. 8 (Type I coupling), FIG. 9 (Type II coupling), FIGS. 14 and 15 (immobilized affinity probe).

In another aspect, the invention provides a detection system that includes at least one unimolecular probe as disclosed herein. In one embodiment, such a probe includes at least one and preferably all of the following as operably linked components:
a) at least one nucleic acid based signal transducer comprising a single-stranded nucleic acid sequence which sequence includes a first object sequence and a first complement sequence in which the first object and first complement sequences are each independently between about 3 to about 150 nucleotides in length, preferably about 4 to about 100 nucleotides in length, more preferably between from about 4 to about 50 nucleotides in length, the first object and first complement sequences being substantially complementary (i.e., over at least 80%, 90% or greater of their respective lengths) to each other so as to form an “off” hybridization state in the absence of the target agent and an “on” hybridization state in the presence of the target agent;
b) at least one recognition element operably linked to the nucleic acid signal transducer, wherein the recognition element specifically interacts with at least one target agent; and
c) optionally, at least one detectable label operably linked to the probe, preferably the nucleic acid signal transducer, wherein interaction of the target agent to the probe, particularly the recognition element conformationally shifts the “off” hybridization state to the “on” hybridization state, the detectable label producing a characteristic signal whose level is indicative of the amount of the “on” hybridization state of the transducer.

In embodiments in which a competitive detection format is desired, such a probe will further include at least one pair of complementary nucleic acid sequences consisting of a second object sequence and a second complement sequence.

A characteristic signal is “altered” according to the invention (increased or decreased) if there is a signal outputted by the target detection system is detectable when compared to a suitable control. Examples of suitable controls include addition to the system of a volume of water, buffer, or the like in lieu of the target agent to be detected. It will be apparent to those with skill in this field that once the output signal associated with a particular control (if any) is understood, it may not be necessary to perform a control for each invention use. This feature of the invention can be important in applications that require multiple testing for the same or similar target agent.

One embodiment of a non-competitive probe according to the invention includes as operably linked components: 1) a first pair of nucleic acid sequences consisting of a first object sequence and a first complement sequence; 2) at least one recognition element conjugated to at least one of the first object and first complement sequences; and, optionally, 3) at least one detectable label. The recognition element is an element that can specifically interact with one or more target agents. Specific identity of the recognition element varies depending on the nature of the target agent as will be discussed in detail when describing each type of the probes. The first object and first complement sequences typically span about 3 to about 150 nucleotides, preferably about 4 to about 100 nucleotides, more preferably about 4 to 50 nucleotides, and are substantially complementary to each other. By the phrase “substantially complementary” is meant that the object and complement sequences can form a hybridized duplex either in the presence or absence of the target agent under the conditions (including a detection temperature) of an assay using the probe, and in addition, at least one of the object and complement sequences may have one or more sequences needed for the purpose of detection e.g., those needed for the PCR or polymerization reaction signal amplification strategies discussed above. Under appropriate conditions, the first object and first complement sequences form a first hybridized duplex having about 3 to about 100 complementary base pairs, preferably about 4 to about 70, and usually about 4 to about 50 of such base pairs.

The non-competitive probe is designed to undergo a conformational change from a hybridized conformation in which the first hybridized duplex is formed, to a dissociated conformation in which the first hybridized duplex is dissociated, or vice versa depending on the presence or absence of the target agent. Since nucleic acid hybridization is a reversible thermodynamic phenomenon, by the phrase “conformational change” is meant by a change in the probability distribution between the two conformations.

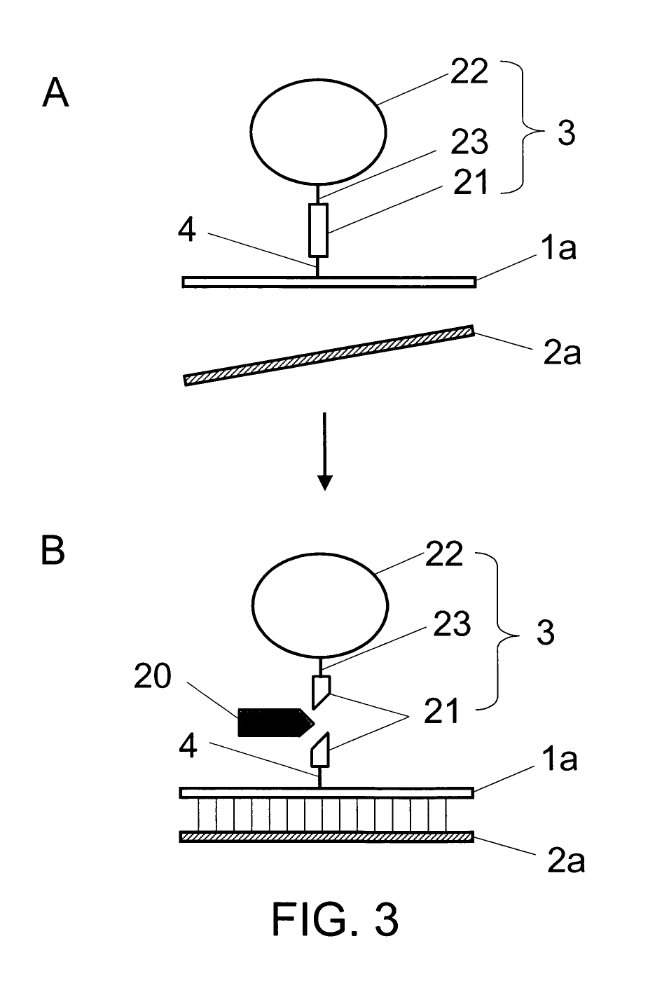

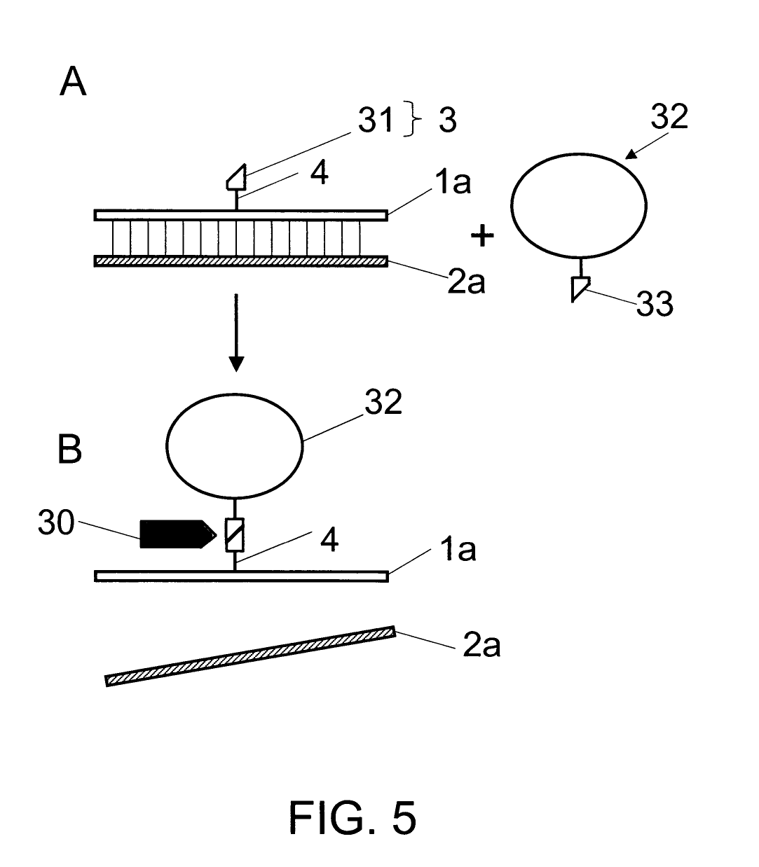

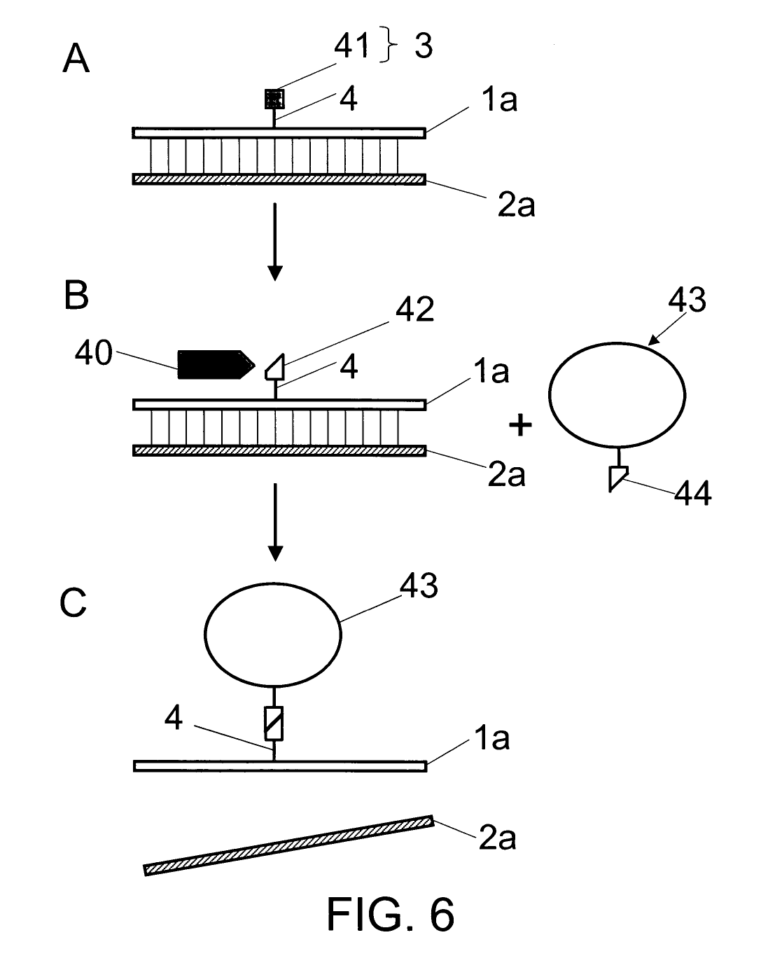

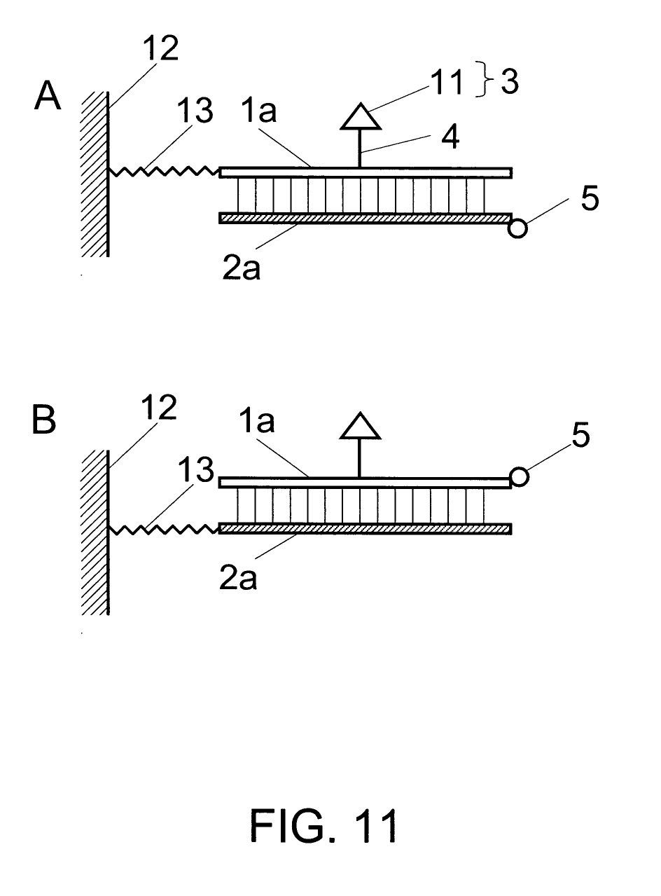

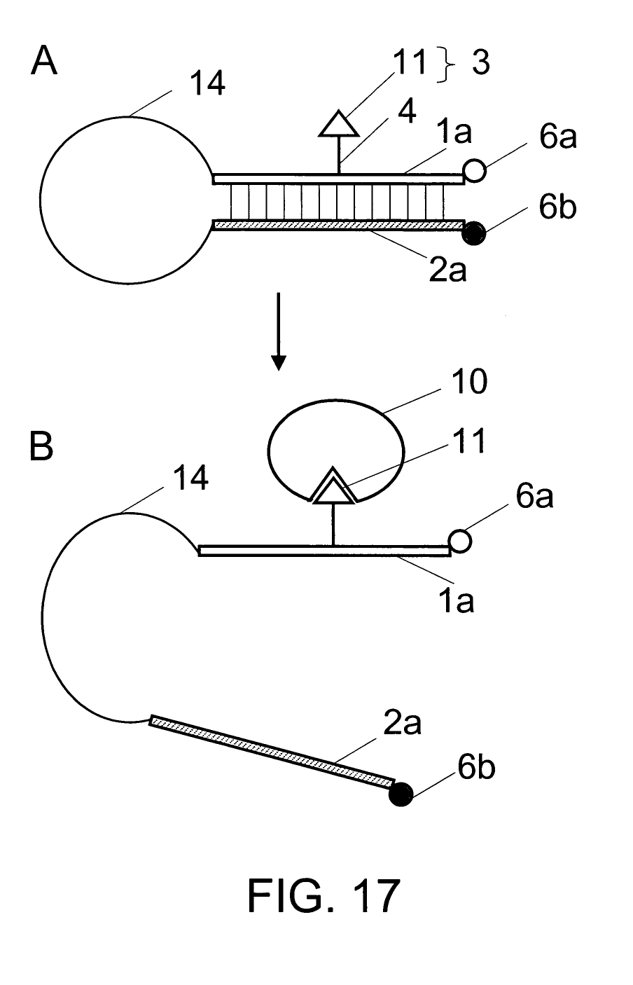

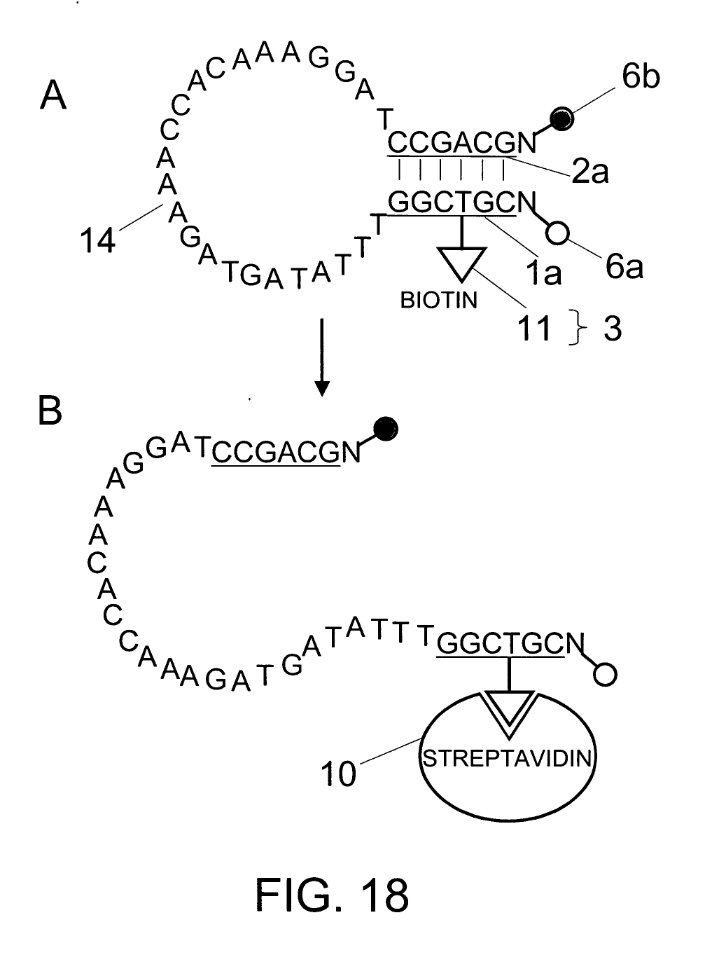

Examples of such non-competitive probes have been provided in the Drawings. See FIGS. 1, 10, 16, 20, and 21 (affinity probes using a variety of labeling strategies), FIG. 3 (cleavage), FIG. 5 (Type I coupling), FIG. 6 (Type II coupling), and FIG. 11 (immobilized affinity probe). FIGS. 17 and 18 show specific examples of a non-competitive probe in a unimolecular format using an interactive label pair.

Additionally suitable probes for use in a competitive format can further include a second pair of competing nucleic acid sequences consisting of a second object sequence and a second complement sequence. The second object and second complement sequences have about 3 to about 150 nucleotides, preferably about 4 to about 100 nucleotides, and more preferably about 4 to about 50 nucleotides, and are substantially complementary to each other so that they can form a second hybridized duplex having about 3 to about 100 complementary base pairs, preferably about 4 to about 70, and usually about 4 to about 50 of such base pairs under appropriate conditions. The first and second object sequences are usually contained in a same nucleic acid sequence, namely an object sequence, and have an overlapping region consisting of at least one nucleotide in common. In one preferred embodiment, the overlapping region includes at least about 10%, preferably about 20%, more preferably about 30% of nucleotides included in the first object sequence. Therefore, the first and second complement sequences hybridize competitively with the object sequence to form either the first hybridized duplex or the second hybridized duplex. Because of this competitive nature of the hybridization, formation of one hybridized duplex prohibits or sufficiently suppresses formation of the other hybridized duplex.

Typical competitive probes in accord with the invention are designed to undergo a conformational change from a first hybridized conformation in which the first hybridized duplex is formed, to a second hybridized conformation in which the second hybridized duplex is formed, or vice versa depending on the presence or absence of the target agent. By the phrase “conformational change” is meant a detectable change in the probability distribution between the two hybridized conformations.

In certain invention embodiments, at least one detectable label is conjugated to the probe to generate a characteristic signal that is indicative of the amount of the target agent or degree of the interaction between the target agent and the recognition element. In most non-competitive probes, the characteristic signal from the detectable label is a function of the degree of the hybridization between the first object and first complement sequences, more specifically a function of the amount of the hybridized or dissociated conformation. In the competitive probes, the characteristic signal from the detectable label is a function of the amount of the first or second hybridized conformation. Various construction methods for incorporating the detectable label will be described below.

As mentioned previously, typical probes of the invention include at least one recognition element operably linked thereto, preferably less than about five of same, and usually about one or two recognition elements. In most embodiments, each of the at least one recognition element is conjugated to at least one of the first object and first complement sequences. By “conjugated”, “conjugation” and the like is meant at least one covalent (e.g., carbon-carbon, carbon-oxygen bonding, etc.) or non-covalent (e.g., hydrogen bonding, ionic bonds, salt bridges, etc.) bond between the nucleic acid based signal transducer and the recognition element. For many embodiments, covalent conjugation will be generally preferred. Examples of particular non-covalent interactions include, but not limited to, hydrogen bonding, ionic bonds and hydrophilic and hydrophobic interactions. Typically, each recognition element is conjugated to the signal transducer by one or more coupling elements. Examples of suitable coupling element include chemical bonds, divalent atoms, divalent chemical moieties, and multivalent chemical moieties.

In some embodiments of the present probes, two or more recognition elements may be conjugated to the nucleic acid based signal transducer by a multivalent coupling element such as a dendrimer.

In other embodiments, a recognition element according to the invention may be conjugated using more than one coupling elements e.g., less than ten, preferably between from about 1 to about 5, more preferably about two of same. The recognition element may be conjugated to nearly any location in the probe e.g., the first object or the first complement sequence using nearly any type of coupling elements provided the desired conformational change in the transducer can be induced. As discussed, the nucleic acid based signal transducer is conformationally sensitive and responds to interaction between the target agent and the recognition element.

In one competitive probe format, it is preferred to conjugate the recognition elements to at least one of the first object and first complement sequences at a location other than the overlapping region included in the first object sequence. If desired, the probe is readily adapted to detect more than one, preferably less than about 3 target agents by conjugating a plurality of different recognition elements in one probe.

As discussed, it is an object of this invention to provide probes that are fully responsive to “a destabilization effect” that is typically induced (or eliminated) as a result of the interaction between the recognition element and the target agent. That interaction, when productive, facilitates a conformational change in the probe and particularly the nucleic acid based signal transducer. In one probe embodiment, interaction of the target agent with the recognition element modulates (increases or decreases) thermodynamic stability of the first hybridized duplex of the transducer. However in competitive probes having two competing pairs of the object and complement sequences, it is preferred that the second hybridized duplex be under no or weaker influence of the interaction compared to the first hybridized duplex. Typically, this is achieved by conjugating the recognition element to a location in the first object sequence or the first complement sequence excluding the overlapping region included in the first object sequence. The interaction could destabilize the first hybridized duplex or release the first hybridized duplex from a destabilized state depending on the type of the probe as will be discussed. Preferably, the melting temperature of the first hybridized duplex changes at least about 1° C., and more preferably at least about 5° C. by the interaction of the target agent.

Particular probes of the invention report presence of one or more target agents through a probe conformation destabilizing (or in some instances stabilizing) effect that is associated with interaction of one or more target agents with the recognition element. More specifically, the probe conformation destabilizing (or stabilizing) effect is typically manifested by a shift in conformation of the nucleic acid based signal transducer. The effect can be transferred to the transducer directly or in some embodiments, indirectly through the recognition element. For instance, in one embodiment, the destabilizing effect accompanying target agent interaction with the probe can be facilitated by steric hindrance brought about by a destabilizing agent that is attached to, or released from the recognition element by the action of the target agent. However, other types of hindrance are envisioned such as those resulting from electrostatic interactions, dipole-dipole interaction, hydrophilic and hydrophobic interactions (e.g., van der Waals forces), electron redistribution, etc. are also possible. Existence of the steric hindrance (or other types of hindrance) can, in some probe embodiments, induce structural distortion of the first hybridized duplex, thereby causing it thermodynamically unstable or less stable compared to the second hybridized duplex under the assay conditions including a detection temperature.

Thus by the phrase “destabilizing agent” is meant nearly any molecule (naturally-occurring, synthetic or semi-synthetic) that can induce or help induce a detectable shift in the conformation of the nucleic acid based signal transducer. The size and character of a destabilizing agent will vary depending on intended use of the target system. For instance, certain proteins or protein complexes can be used such as those having a size of about 1 to about 25 nm in diameter, typically about 5 to about 15 nm in diameter. Destabilizing agents find particular use in combination with the cleavage and coupling probes described herein. See FIGS. 3 and 5, for instance.

Detailed explanation is given below for each type of the probes referring to the figures.

FIG. 1 schematically shows a non-competitive version of an affinity probe comprising the first pair of the nucleic acid sequences, without the detectable label. In the affinity probe, the recognition element 3 is a probe ligand 11 that can specifically bind to a receptor agent 10. According to the invention, one or more probe ligands can be conjugated to any location in the first object sequence 1a or the first complement sequence 2a. Each probe ligand is conjugated by at least one coupling element 4. In this figure, the recognition element 3 is a probe ligand 11 that can specifically bind to a target “receptor” agent 10. The adaptor in this example of the invention would correspond to the coupling element 4 which couples (or links) the probe ligand (i.e., the recognition element) to the nucleic acid signal transducer.

By the word “adaptor” is meant a coupling element, particularly when the probe is an affinity probe. However for cleavage type probes, the adaptor includes the coupling element, at least one cleavage site, and linker. In other embodiments in which Type I, II or II(-) coupling probes are used, the preferred adaptor will include the coupling element.

The non-competitive version of the affinity probe is designed to undergo a conformational change from the hybridized conformation (FIG. 1A) to the dissociated conformation (FIG. 1B) upon binding of the receptor agent 10 to the probe ligand 11. Specifically, the conformational change induced by the binding of the receptor agent, causes decrease of the probability in the hybridized conformation and increase of the probability in the dissociated conformation.

In order to induce the desired conformational change, binding of the receptor agent must destabilize the first hybridized duplex sufficiently strongly. It is preferred that the melting temperature of the first hybridized duplex be lowered at least about 1° C., more preferably at least about 5° C. upon binding of the receptor agent.

In preferred embodiments, the hybridized conformation is thermodynamically stable in the absence of the receptor agent under the assay conditions including a detection temperature. Typically, the preferred affinity probe in this version has a melting temperature of the first hybridized duplex in the absence of the receptor agent higher than the detection temperature, preferably at least about 5° C. higher, and more preferably at least about 10° C. higher than the detection temperature.

FIG. 2 shows a competitive version of an affinity probe, which further comprises the second pair of the competing nucleic acid sequences, without the detectable label. In preferred embodiments, the probe ligand 11 is conjugated to the first object sequence 1a excluding the overlapping region 1c, or to the first complement sequence 2a. As discussed, the first and second object sequences 1a, 1b are contained in the object sequence 1. This version of the probe is designed to undergo a conformational change upon binding of the receptor agent 10, from a first hybridized conformation (FIG. 2A) in which the first hybridized duplex between the first object sequence 1a and the first complement sequence 2a is formed, to the second hybridized conformation (FIG. 2B) in which a second hybridized duplex between the second object sequence 1b and the second complement sequence 2b is formed. Specifically, the conformational change induced by the binding of the receptor agent, causes decrease of the probability in the first hybridized conformation and increase of the probability in the second hybridized conformation.

In order to induce the desired conformational change, binding of the receptor agent typically must destabilize the first hybridized duplex sufficiently strongly, while sufficiently weakly or not destabilizing the second hybridized duplex. It is preferred that the melting temperature of the first hybridized duplex be lowered at least about 1° C., more preferably at least about 5° C. upon binding of the receptor agent.

In preferred embodiments, the first hybridized duplex is preferentially formed compared to the second hybridized duplex in the absence of the receptor agent. Preferably, the melting temperature of the first hybridized duplex is at least about 1° C. higher, more preferably at least about 5° C. higher than that of the second hybridized duplex in the absence of the receptor agent. It is further preferred that the preferentiality of the hybridization be changed in favor of the second hybridized duplex upon binding of the receptor agent. Preferably, the melting temperature of the second hybridized duplex is at least about 1° C. higher, more preferably at least about 5° C. higher than that of the first hybridized duplex in the presence of an excess of the receptor agent.

In preferred embodiments, both the first and second hybridized duplexes are thermodynamically stable in the absence of the receptor agent under the assay conditions including a detection temperature. Typically, the preferred affinity probe has melting temperatures of the first and second hybridized duplexes in the absence of the receptor agent higher than the detection temperature, preferably about 5° C. higher, and more preferably at least about 10° C. higher than the detection temperature.

In the affinity probes described above, the destabilization effect caused by the receptor binding is due to in most cases steric hindrance between the receptor agent (or the receptor agent-probe ligand complex) and the first hybridized duplex, although other types of hindrance resulting from electrostatic interaction, dipole-dipole interaction, hydrophilic and hydrophobic interactions (e.g., van der Waals interactions), electron redistribution, etc. are also possible.

In order to induce a larger steric hindrance, it will be often preferred that the receptor agent be larger and located closer to the nucleic acid sequence to which the probe ligand is conjugated. Often, the approximate size of the receptor agent will be known because it is a target agent to be detected. When the receptor agent is too small to induce a sufficiently strong steric hindrance, more than one probe ligand may be conjugated by using a multivalent chemical moiety such as a dendrimer as a coupling element. In preferred embodiments, it is desired to control the distance between the receptor agent (or the receptor agent-probe ligand complex) and the first object and/or first complement sequences by changing the length of the coupling element. Use of a shorter coupling element would be preferred because steric hindrance (and also other types of hindrance) gets stronger in shorter distance. However, too short coupling element could prohibit or suppress binding of the receptor agent to the probe ligand. Therefore, the length of the coupling element and other parameters must be selected appropriately to maximize the destabilization effect as well as the binding of the receptor agent.

As will be apparent, use of the present invention is compatible with the detection and in some instances identification of a wide spectrum of target receptor agents. Illustrative of such receptor agents include those receptors, antibodies, and enzymes that are less than about 50 nm in diameter, preferably between about 1 to about 25 nm, and more preferably about 10 nm in diameter with about 5 to about 15 nm in diameter being a typically useful range. Substrates and inhibitors of enzymes or carbohydrates may be somewhat smaller e.g., a few nm or smaller in length or diameter. If the receptor agent is a virus or microorganism having receptor molecules on its surface, the size of the receptor agent can be in the range of about 100 nm to a few μm (or up to about 10 μm).

A wide variety of adaptors and more specifically coupling elements can be used in accord with the present invention. In general, one preferred coupling element length (i.e., the distance between the receptor agent (or the destabilizing element) and the nucleic acid signal transducer) will depend in large part on the size and shape of the receptor agent or the destabilizing agent to be used. However, in most instances, the length of the coupling element will be suitable for use with the invention if it is essentially the same size as or somewhat shorter than the size of the receptor agent (or the destabilizing agent), preferably shorter than about half or quarter of the size of the receptor agent (or the destabilizing agent). Without wishing to be bound to theory, it is believed that at least some portion of the receptor agent (or the destabilizing agent), especially irregularly extruded portions, will in some cases make contact with the nucleic acid based signal transducer e.g., in the course of the rotational or rocking (or other vibrational) motions of the receptor agent. In some instances, this motion can facilitate the destabilizing force that assists conformational switching of the nucleic acid based signal transducer of the invention.

More specific coupling elements suitable for use with the probes described herein include those linker and related structures that are flexible and freely rotatable. Examples include carbon chains (preferably straight chain) such as an optionally substituted alkyl, alkenyl, or alkynyl chain, a polypeptide (or peptide) chain, a peptide mimetic or analogue, a nucleic acid chain, a synthetic polymer or co-polymer chain and the like such as a polyethylene glycol chain and a polyester chain. This type of coupling elements may be preferred in instances where extensive freedom of movement between the receptor agent (or the destabilizing agent) and the nucleic acid based signal transducer is preferred.

By the phrase “optionally substituted” is meant substitution by other than hydrogen at one or more available positions, typically 1 to 3 or 4 positions, by one or more suitable groups such as those disclosed herein.

Suitable groups that may be present on a “substituted” group, moiety or other site as disclosed herein include halogen such as fluoro, chloro, bromo and iodo; cyano; hydroxyl; nitro; azido; alkanoyl such as a C₁₋₆ alkanoyl group such as acyl and the like; carboxamido; alkyl groups including those groups having 1 to about 12 carbon atoms, or 1, 2, 3, 4, 5, or 6 carbon atoms; alkenyl and alkynyl groups including groups having one or more unsaturated linkages and from 2 to about 12 carbon, or 2, 3, 4, 5 or 6 carbon atoms; alkoxy groups having those having one or more oxygen linkages and from 1 to about 12 carbon atoms, or 1, 2, 3, 4, 5 or 6 carbon atoms; aryloxy such as phenoxy; alkylthio groups including those moieties having one or more thioether linkages and from 1 to about 12 carbon atoms, or 1, 2, 3, 4, 5 or 6 carbon atoms; alkylsulfinyl groups including those moieties having one or more sulfinyl linkages and from 1 to about 12 carbon atoms, or 1, 2, 3, 4, 5, or 6 carbon atoms; alkylsulfonyl groups including those moieties having one or more sulfonyl linkages and from 1 to about 12 carbon atoms, or 1, 2, 3, 4, 5, or 6 carbon atoms; aminoalkyl groups such as groups having one or more N atoms and from 1 to about 12 carbon atoms, or 1, 2, 3, 4, 5 or 6 carbon atoms; carbocyclic aryl having 6 or more carbons, particularly phenyl (e.g., an R group being a substituted or unsubstituted biphenyl moiety); aralkyl having 1 to 3 separate or fused rings and from 6 to about 18 carbon ring atoms, with benzyl being a preferred group; aralkoxy having 1 to 3 separate or fused rings and from 6 to about 18 carbon ring atoms, such as 0-benzyl; or a heteroaromatic or heteroalicyclic group having 1 to 3 separate or fused rings with 3 to about 8 members per ring and one or more N, O or S atoms, e.g., coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl.

In certain invention embodiments more structured coupling elements i.e., less freedom of movement, such as an optionally substituted alkenyl or alkynyl chains, or use of more than one coupling elements linked together to a recognition element which do not allow as much free rotation or rocking (or other vibrational) motions may be more preferred. Additional coupling elements include those that include or consist of dendrimers. Of course, the flexible and freely rotatable coupling elements already described can be made less flexible by incorporating one or more optionally substituted ring systems e.g., phenyl, benzyl, etc. Without wishing to be bound to theory, it is believed that in some embodiments, certain receptor agents (or the destabilizing agents) can be locked into a strongly destabilizing conformation with respect to the nucleic acid signal transducer by using coupling elements having less freedom of movement. A suitable coupling element chain length in accord with the invention (or the distance between the receptor agent (or the destabilizing element) and the nucleic acid signal transducer) will generally be between from about 1 nm to about 100 nm depending often on the size of the receptor agent, most typically about 1 nm to about 10 nm.

Other considerations will be useful in determining a preferred coupling element length suited to a particular invention application. More specifically, in some embodiments, a typically preferred length of the coupling element can be similar to or slightly larger than the depth scale of the binding site of the receptor agent (or the destabilizing agent). For example, in embodiments, the binding site of an antibody target agent is known to be about 0.5 to about 3.5 nm deep, and the binding site of the receptors and enzymes is typically about 0.5 to about 5 nm deep. It will be appreciated that there is general understanding that repulsion due to van der Waals interaction typically occurs in the distance range about 0.3 to about 0.5 nm, charge-charge or electrostatic repulsion occurs in a longer distance range (say up to a few nm or longer), and nuclear charge repulsion occurs in a shorter distance range (say about 0.1 nm or shorter). Accordingly, and without wishing to be bound to theory, a preferred length of the coupling element will range from about 1 nm (or slightly shorter) to about 10 nm, or preferably about 1 nm to about 5 nm.

When the structure of the receptor agent-probe ligand complex are known in the art or else it can be calculated by the aid of computer simulation, preferred length or structure of the coupling element could be predicted or estimated. However, because the behavior of the probe in the complex assay solutions cannot always be predicted in certainty, empirical testing is very useful in optimizing the probe according to the invention.

The affinity probes according to the invention are useful for detecting various receptor agents. In the present invention, the terms “receptor” and “ligand” are used with broadly to encompass one part of a binding pair. By the term “receptor or receptor agent” as used herein is meant nearly any molecular entity which specifically binds to a complementary molecular entity that is referred to herein as a “ligand” such as a probe ligand. Examples of the receptor agents include, but are not limited to, proteins, glycoproteins, polypeptides, carbohydrates, lipids, phospholipids, nucleic acids, antibodies, antibody fragments, antigens, enzymes, receptors, hormones, cytokines, viruses, bacteria, microorganisms, and the like including functional fragments thereof. Examples also include receptor agents from other than biological sources, such as supramolecules (e.g., crown ethers or similar compounds) and nanoparticles. Examples of the probe ligands include chemical and biochemical ligands, antigens, antibodies, antibody fragments, enzymes, substrates or inhibitors of enzymes, hormones, antibiotics, narcotics, toxins, polypeptides, proteins, protein fragments, targeting sequences or transit peptides, glycoproteins, lipids, phospholipids, polysaccaharides, carbohydrates, nucleic acids, peptide nucleic acids, and the like. Nearly any kind of ligands can be used as probe ligands as long as there is specificity in the binding of the receptor agent to the probe ligand and also such binding causes the desired conformational change. Additional consideration for conjugating the probe ligand is that conjugation of the probe ligand must not prohibit or substantially interfere the binding of the receptor agent.

FIG. 3 shows a non-competitive version of a cleavage probe without the detectable label. In the cleavage probe, the recognition element 3 is a probe substrate comprising at least one cleavage site 21 specific to a reaction-inducing agent 20 and a destabilizing agent 22. One end of the at least one cleavage site is conjugated to the destabilizing agent by a linker 23, typically via a covalent or non-covalent linkage. Examples of the link include chemical bonds, divalent atoms, divalent chemical moieties, and multivalent chemical moieties. The other end of the cleavage site may be conjugated to any location in the first object sequence 1a or the first complement sequence 2a by the coupling element 4. According to the invention, more than one probe substrates (i.e., recognition elements) may be conjugated.

By the phrase “reaction-inducing agent” is meant a cleaving molecule, usually a chemical or protein sequence such as an enzyme that can hydrolyze at least part of the recognition element. Such hydrolysis can be reversible or irreversible to break the cleavage site, for instance, and/or other portion of the recognition element. Examples of preferred enzymes generally include proteases, endonucleases, lipases, glycosidases and the like as described herein. Illustrative chemical reaction-inducing agents are provided below.

In this invention example, the reaction-inducing agent specifically cleaves the cleavage site and releases the destabilizing agent that destabilizes the first hybridized duplex. Therefore, the non-competitive cleavage probe is designed to undergo a conformational change from a dissociated conformation (FIG. 3A) to a hybridized conformation (FIG. 3B) upon cleavage of the cleavage site by the reaction-inducing agent. Specifically, the conformational change induced by the cleavage, facilitates an increase of the probability in the hybridized conformation and decrease of the probability in the dissociated conformation.

In order to induce the desired conformational change, the destabilizing agent must destabilize the first hybridized duplex sufficiently strongly. Nearly any type of destabilizing agent can be used as long as a sufficiently strong destabilizing effect is driven. It is preferred to use a protein or a protein complex having a large size as a destabilizing agent, because steric hindrance could be readily driven due to the large size. In one preferred embodiment, streptavidin or its derivative bound to biotin in which biotin is linked to one end of the cleavage site may be used as a destabilizing agent. Chemistry for linking biotin is well known in the art. In preferred cleavage probes, the melting temperature of the first hybridized duplex is raised at least about 1° C., more preferably at least about 5° C. upon cleavage.

In preferred embodiments, the hybridized conformation is thermodynamically unstable in the absence of the reaction-inducing agent under the assay conditions including a detection temperature. Typically, the preferred cleavage probe in this version has a melting temperature of the first hybridized duplex in the absence of the reaction-inducing agent lower than the detection temperature, preferably at least about 5° C. lower, and more preferably at least about 10° C. lower than the detection temperature.

FIG. 4 shows a competitive version of a cleavage probe, which further comprises the second pair of the competing nucleic acid sequences. In preferred embodiments, the cleavage site 21 is conjugated to the first object sequence 1a excluding the overlapping region 1c, or to the first complement sequence 2a. The competitive cleavage probe is designed to undergo a conformational change upon cleavage of the cleavage site, from a second hybridized conformation (FIG. 4A) in which the second hybridized duplex is formed, to a first hybridized conformation (FIG. 4B) in which the first hybridized duplex is formed. Specifically, the conformational change induced by the cleavage, causes increase of the probability in the first hybridized conformation and decrease of the probability in the second hybridized conformation.

In order to induce the desired conformational change, the destabilizing agent must destabilize the first hybridized duplex sufficiently strongly, while sufficiently weakly or not destabilizing the second hybridized duplex. It is preferred that the melting temperature of the first hybridized duplex be raised at least about 1° C., more preferably at least about 5° C. upon cleavage of the cleavage site.

In preferred embodiments, the second hybridized duplex is preferentially formed compared to the first hybridized duplex in the absence of the reaction-inducing agent. Preferably, the melting temperature of the second hybridized duplex is at least about 1° C. higher, more preferably at least about 5° C. higher than that of the first hybridized duplex in the absence of the reaction-inducing agent. It is further preferred that the preferentiality of the hybridization be changed in favor of the first hybridized duplex upon cleavage. Preferably, the melting temperature of the first hybridized duplex is at least about 1° C. higher, more preferably at least about 5° C. higher than that of the second hybridized duplex in the presence of an excess of the reaction-inducing agent.

In preferred embodiments, the second hybridized duplex is thermodynamically stable but the first hybridized duplex is unstable or less stable in the absence of the reaction-inducing agent under the assay conditions including a detection temperature. Typically, the preferred cleavage probe in this embodiment has the melting temperature of the second hybridized duplex in the absence of the reaction-inducing agent higher than the detection temperature, preferably at least about 5° C. higher, and more preferably at least about 10° C. higher than the detection temperature.

In the cleavage probes described above, distance between the destabilizing agent and the nucleic acid sequence to which the probe substrate is conjugated may be controlled (optimized) to induce a sufficiently strong destabilization effect. In most invention embodiments, a shorter distance would give stronger destabilization. Use of a larger destabilizing agent would induce stronger destabilization. However, too short a distance or too large a size may prohibit or suppress the activity of the reaction-inducing agent. Therefore, such parameters must be selected appropriately to maximize the destabilization effect as well as the activity of the reaction-inducing agent.

Particular cleavage probes according to the invention are useful for detecting reaction-inducing agents having specific activities for cleaving the cleavage sites. As has been mentioned, the invention is flexible and is not limited to use of any particular cleavage site. Examples of the cleavage sites include, but not limited to, those specific to various enzymes having endogeneous cleavage activities such as proteases, endonucleases, lipases, and glycosidases. Chemical cleaving reagents such as cyanogen bromide (CNBr) and hydroxylamine (NH₂—OH) that are known to specifically cleave particular peptide bonds are also envisioned. Identity of the cleavage site varies depending on the reaction-inducing agent. For instance, the cleavage site will be an amino acid sequence for a protease, a double stranded DNA sequence for an endonuclease, a lipid for a lipase, and a carbohydrate for a glycosidase.

A variety of specific cleavage sites in accord with the invention are described below. For instance, the cleavage site specific to human heparanase-1 is an important example. Heparanase-1 is an endoglycosidase that is thought to be involved in metastasis of tumor cells. It has been recognized that heparanase-1 mediates penetration of tumor cells across the tissue barrier by causing degradation of extracellular matrix. Examples of oligosaccharide cleavage sites specific to human heparanase-1 are listed in Table 1.

TABLE 1

Examples of oligosaccharide cleavage sites
specific to human hepananase-1⁽¹⁾

Cleavage site⁽²⁾

ΔHexUA-GlcN(NS,6S)-GlcUA/GlcN(NS,6S)-GlcUA-
GlcN(NS,6S)
ΔHexUA(2S)-GlcN(NS,6S)-GlcUA/GlcN(NS,6S)-GlcUA-
GlcN(NS,6S)
ΔHexUA(2S)-GlcN(NS,6S)-GlcUA/GlcN(NS,6S)
GlcUA(2S)-GlcN(6S)-GlcUA/GlcN-GlcUA(2S)-GlcN(3S)

⁽¹⁾References: Okada Y. et al. (2002) and Dempsey L. A. et al. (2000).
⁽²⁾ΔHexUA, GlcN, and GlcUA represent unsaturated hexuronic acid, glucosamine, and glucuronic acid, respectively. 2S, 3S, 6S, and NS represent 2-O, 3-O, 6-O, and 2-N-sulfation, respectively. Position of the cleavage is denoted by /.

Protease-specific cleavage sites are also very important because many of mammalian or viral proteases are involved in human diseases.

Additionally preferred protease cleavage sites are those that are specifically hydrolyzed by a protease associated with a human pathogen e.g., yeast, bacterium, fungus, nematode, virus or protozoan. More specific examples include cytomegalovirus (CMV); herpes simplex virus (HSV); hepatitis virus, preferably type A or C; human immunodeficiency virus (HIV), Kaposi's sarcoma-associated herpes virus (KSHV), yellow fever virus, flavivirus, rhinovirus, or a plasmodium such as P. falciparum, P. vivax, P. ovale, or P. malariae. Typically, the plasmodia cause malaria or various medical complications relating to malaria. There is recognition that the proteases plasmepsin I and plasmepsin II are implicated. In embodiments in which HSV is of interest, the protease will be the maturational protease of HSV.

A variety of particular HIV-1 and HCV protease specific cleavage sites have been disclosed. See e.g., Gluzman, I. Y. et al., J. Clin. Invest., 94:1602 (1994); Grakoui, A. et al., J. of Virol., 67:2832 (1993); Kolykholov, A A. et al., J. of Virol., 68:7525 (1994); and Barrie, K. A. et al., Virology, 219:407 (1996), the disclosures of which are incorporated by reference.

Additional pathogen-specific proteases and specified cleavage sites have been described and can be used in accord with the present invention. For example, an HSV-1 maturational protease and protease cleavage site has been described. See e.g., Hall, M. R. T. and W. Gibson, Virology, 227:160 (1997). Further, the plasmepsins I and II have been found in the digestive vacuole of P. falciparum. The corresponding protease cleavage sites have also been disclosed. See e.g., Moon, R. P., Eur. J. Biochem., 244:552 (1997).

Additional protease specific cleavage sites for use with the invention are specifically cleaved by a mammalian protease associated with blood coagulation, apoptosis, Alzheimer's disease, or the extracellular matrix.

Examples of protease-specific cleavage sites are listed in Table 2.

TABLE 2
Examples of protease-specific cleavage sites

| | | | |
|---|---|---|---|
| | | SEQ ID | Genbank |
| Protease | Cleavage site⁽¹⁾ | NO | Accession No. |

| | | | | |
|---|---|---|---|---|
| Viral | HIV-1 | SQNY/PIV | 1 | K03455 |
| proteases | protease | ARVL/AEA | 2 | |
| | | ATIM/MQR | 3 | |
| | | RQAN/FLG | 4 | |
| | | PGNF/LQS | 5 | |
| | | SV/PQI | 6 | |
| | | SFNF/PQI | 7 | |
| | | TLNF/PIS | 8 | |
| | | AETF/YVD | 9 | |
| | | RKVL/FLD | 10 | |
| | HCV Type I | DEMEEC/ATHL | 11 | AF333324 |
| | protease | CSTPCS/GSWL | 12 | |
| | | EDVVCC/SMSY | 13 | |
| | | EDVVPC/SMSY | 14 | D16435 |
| | Type II | DEMEECASHL | 15 | |
| | protease | MQTTCP/CGAQ | 16 | |
| | | DDIVCC/SMSY | 17 | |
| | HSV-1 | LVLA/S | 18 | X14112 |
| | protease | LVLA/SS | 19 | |
| | HTLV-1 | SAPQVLPVMHPN | 20 | L36905 |
| | protease | SKTKVLVVQPKN | 21 | |
| | HCMV | VVNA/S | 22 | P16753 |
| | assemblin | VVNA/SS | 23 | |
| Mammalian | alpha- | VHHQKLV/F/FAEDVGSNK | 24 | NM_000484 |
| proteases | secretase | | | |
| | beta-secretase | SEVKM/DAEFR | 25 | NM_000484 |
| | | SEVNL/DAEFR | 26 | |
| | gamma- | RRGGVV/IA/TVIVGER | 27 | NM_000484 |
| | secretase | | | |
| | plasmin | PRFK/IIGG | 28 | |
| | MMP-1 | PLG/L | 29 | |
| | | PLG/I | 30 | |
| | MMP-3 | IPEN/FFGV | 31 | NM_013227 |
| | | GPEG/LRVG | 32 | |
| | | RVGF/YESD | 33 | |
| | | LLSA/LVET | 34 | |
| | | EAIP/MSIP | 35 | |
| | | RAIH/IQAE | 36 | |
| | | PFSP/LVAT | 37 | |
| | aggrecanase | TEGE/ARGS | 38 | |
| | caspase-1 | YVHD/A | 39 | |
| | | DGPD/G | 40 | |
| | | DEVD/G | 41 | |
| | caspase-2 | DEVD/G | 41 | |
| | caspase-3 | DEVD/G | 41 | |
| | | DGPD/G | 40 | |
| | | DEVD/N | 42 | |
| | | DMQD/N | 43 | |
| | | DXXD/ | 44 | |
| | | DEPD/S | 45 | |
| | | DEAD/G | 46 | |
| | | DETD/S | 47 | |
| | | DAVD/T | 48 | |
| | | ESMD/ | 49 | |
| | caspase-4 | LEVD/X | 50 | |
| | | WEHD/X | 51 | |
| | | LEHD/X | 52 | |
| | caspase-5 | WEHD/X | 51 | |
| | | LEHD/X | 52 | |
| | caspase-6 | VEID/N | 53 | |
| | | DEVD/G | 41 | |
| | | DGPD/G | 40 | |
| | caspase-7 | DEVD/G | 41 | |
| | caspase-8 | IETD/X | 54 | NM_004346 |
| | | LETD/X | 55 | |
| | caspase-9 | LEHD/X | 52 | |
| | caspase-10 | IEAD/X | 56 | |
| | angiotensin- | RPPGFSP/FR | 57 | |
| | converting | DDVYIHPF/HL | 58 | |
| | enzyme | | | |
| | platelet | PGPRGPP | 59 | NM_000506 |
| | glycoprotein | | | |
| | V thrombin | | | |
| | factor IX | NLTR↓IVGG | 60 | |
| | thrombin | TVELQGVVP/RGVNL | 61 | |
| | | TVELQGLVP/RGVNL | 62 | |
| | | DFLAEGGGV/RGPRV | 63 | |
| | | KATNATLDP/RSFLL | 64 | |
| Plasmodium | plasmepsin I | ERMF/LSFP | 65 | NM_000558 |
| Protease | | FPHF/DLSH | 66 | |
| | | VNFK/LLSH | 67 | |
| | | LGRL/LVVY | 68 | |
| | | TQRF/FESF | 69 | |
| | | VQAA/YQKV | 70 | |
| | plasmepsin II | ERMF/LSFP | 65 | NM_000558 |
| | | LLVT/LAAH | 71 | |
| | | STVL/TSKY | 72 | |
| | | GRLL/VVYP | 73 | |
| | falcilysin | TTKT/YFPH | 74 | NM_000558 |
| | | AHVD/DMPN | 75 | |
| | | HAHK/LRVD | 76 | |
| | | WTQR/FFES | 77 | |
| | | AFSD/GLAH | 78 | |
| | | LAHL/DNLK | 79 | |
| | | AYQK/VVAG | 80 | |
| Schistoma | cathepsin D | AEAL/ERMF | 81 | |
| Protease | | ERMF/LSFP | 65 | |
| | | FLSF/PTTK | 82 | |
| | | TPEE/KASV | 83 | |
| | | VTAL/WEKV | 84 | |
| | | LGRL/LLVV | 85 | |

⁽¹⁾Position of the cleavage is denoted by / when it is known.

Below, we describe particular examples of Type I, II and II(-) coupling probes according to the invention.

FIGS. 5 and 6 show non-competitive versions of the Type I and II coupling probes without the detectable label, respectively. In the Type I coupling probe shown in FIG. 5, the recognition element 3 is a reaction site 31 to which a destabilizing agent 32 can be conjugated (typically covalently linked) by a specific action of a reaction-inducing agent. The destabilizing agent has at least one reaction group 33 that can form a covalent or non-covalent bond with the reaction site in the presence of the reaction-inducing agent.

In the Type II coupling probe shown in FIG. 6, the recognition element 3 is a reaction site 41 that can be converted specifically to a conjugation site 42 by the action of a reaction-inducing agent 40. The conjugation site is a site to which a destabilizing agent 43 can be conjugated. The destabilizing agent may be a substrate having at least one reaction group 44 that can be covalently or non-covalently linked to the conjugation site either spontaneously or in the presence of a coupling agent, or it may be a receptor agent that can specifically bind to the conjugation site. The coupling or binding of the destabilizing agent must be specific to the conjugation site.

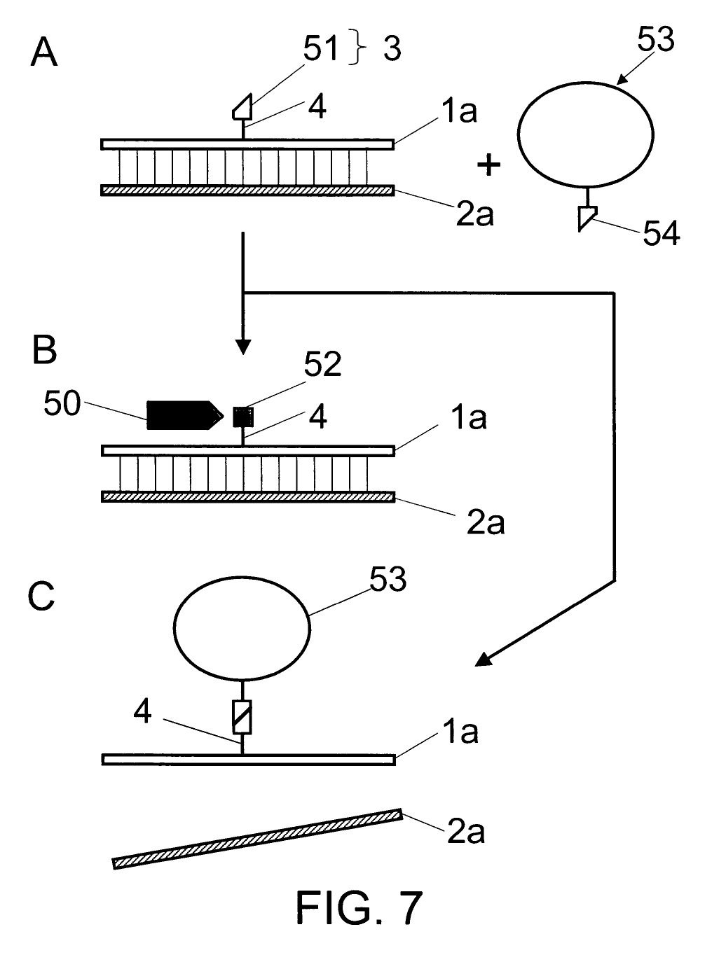

Alternatively, and in addition, the Type II coupling probe may be formatted to include a reaction site that initially acts as a conjugation site to which a destabilizing agent can be conjugated, but is converted to a non-conjugatable site by the action of the reaction-inducing agent. Such a probe will sometimes be referred to herein as a “Type II(-) coupling probe”. In the Type II(-) coupling probe shown in FIG. 7, the destabilizing agent 53 can be conjugated to the probe, particularly to the reaction site 51 (i.e., the recognition site 3), in the absence of the reaction-inducing agent, but it cannot be conjugated to the probe if the reaction site 51 is converted to the non-conjugatable site 52 by the action of the reaction-inducing agent 50. The destabilizing agent 53 may be a substrate having at least one reaction group 54 that can be covalently or non-covalently linked to the conjugatable reaction site 51. Those destabilizing agents that can be used with the Type II coupling probe can also be used with the Type II(-) coupling probe.

In the non-competitive versions of the Type I, II and II(-) coupling probes, one or more reaction sites can be conjugated to any location in the first object sequence 1a or the first complement sequence 2a. Each reaction site is conjugated by the coupling element 4, typically by a covalent linkage.

The Type I and II coupling probes in the non-competitive version are designed to undergo a conformational change from a hybridized conformation (FIGS. 5A and 6A) to a dissociated conformation (FIGS. 5B and 6C) upon coupling or conjugation of the destabilizing agent to the reaction site by the action of the reaction-inducing agent. Specifically, the conformational change induced by the coupling, causes decrease of the probability in the hybridized conformation and increase of the probability in the dissociated conformation.

The Type II(-) coupling probe in the non-competitive version is designed to undergo a conformational change from the dissociated conformation (FIG. 7C) to the hybridized conformation (FIG. 7B) by the action of the reaction-inducing agent, leading to increase of the probability in the hybridized conformation and decrease of the probability in the dissociated conformation.

In order to induce the desired conformational change, the destabilizing agent must destabilize the first hybridized duplex sufficiently strongly. Nearly any type of destabilizing agent can be used as long as a sufficiently strong destabilizing effect is driven. It is preferred to use a protein or a protein complex having a large size as a destabilizing agent, because steric hindrance can be readily driven due to the large size. In one preferred embodiment, streptavidin or its derivative bound to a modified biotin having at least one reaction group for the coupling reaction may be used as a destabilizing agent. Chemistry for modifying biotin is well known in the art. In other preferred embodiments of the Type II and II(-) coupling probe, antibodies, receptors or the like that can specifically bind to the conjugation site (which is the reaction site in the Type II(-) coupling probe) may be used as destabilizing agents. In preferred probes, the melting temperature of the first hybridized duplex is lowered at least about 1° C., more preferably at least about 5° C. upon coupling or conjugation of the destabilizing agent.

In preferred embodiments of the Type I and II coupling probes in the non-competitive version, the hybridized conformation is thermodynamically stable in the absence of the reaction-inducing agent under the assay conditions including a detection temperature. Typically, the preferred Type I and II coupling probes in this version have the melting temperature of the first hybridized duplex in the absence of the reaction-inducing agent higher than the detection temperature, preferably at least about 5° C. higher, and more preferably at least about 10° C. higher than the detection temperature.

In preferred embodiments of the Type II(-) coupling probe in the non-competitive version, the dissociated conformation, when the destabilizing agent is bound to the probe, is thermodynamically stable in the absence of the reaction-inducing agent under the assay conditions including a detection temperature. Typically, the preferred Type II(-) coupling probe in this version has the melting temperature of the first hybridized duplex (with the destabilizing agent bound thereto) in the absence of the reaction-inducing agent lower than the detection temperature, preferably at least about 5° C. lower, and more preferably at least about 10° C. lower than the detection temperature.

FIGS. 8 and 9 show competitive versions of the Type I and II coupling probes, which further comprise the second pair of the competing nucleic acid sequences. In preferred embodiments, the reaction site 31 or 41 is conjugated to the first object sequence 1a excluding the overlapping region 1c, or to the first complement sequence 2a. The Type I and II coupling probes in the competitive version are designed to undergo a conformational change upon coupling of the destabilizing agent, from a first hybridized conformation (FIGS. 8A and 9A) in which the first hybridized duplex is formed, to a second hybridized conformation (FIGS. 8B and 9C) in which the second hybridized duplex is formed. Specifically, the conformational change induced by the coupling of the destabilizing agent 32 or 43, causes decrease of the probability in the first hybridized conformation and increase of the probability in the second hybridized conformation.

According to the present invention, a Type II(-) coupling probe in the competitive version can also be formatted by including a reaction site that initially acts as a conjugation site, but is converted to a non-conjugatable site by the action of the reaction-inducing agent. The Type II(-) coupling probe in the competitive version is designed to undergo a conformational change from the second hybridized conformation to the first hybridized conformation by the action of the reaction-inducing agent. This leads to increase of the probability in the first hybridized conformation and decrease of the probability in the second hybridized conformation.

In order to induce the desired conformational change, the destabilizing agent must destabilize the first hybridized duplex sufficiently strongly, while sufficiently weakly or not destabilizing the second hybridized duplex. It is preferred for the Type I, II and II(-) coupling probes that the melting temperature of the first hybridized duplex be lowered at least about 1° C., more preferably at least about 5° C. upon coupling or conjugation of the destabilizing agent.

In preferred embodiments of the Type I and II coupling probes, the first hybridized duplex is preferentially formed compared to the second hybridized duplex in the absence of the reaction-inducing agent. Preferably, the melting temperature of the first hybridized duplex is at least about 1° C. higher, more preferably at least about 5° C. higher than that of the second hybridized duplex in the absence of the reaction-inducing agent. It is further preferred that the preferentiality of the hybridization be changed in favor of the second hybridized duplex upon coupling. Preferably, the melting temperature of the first hybridized duplex is at least about 1° C. lower, more preferably at least about 5° C. lower than that of the second hybridized duplex in the presence of an excess of the reaction-inducing agent.

In preferred embodiments of the Type II(-) coupling probe, the second hybridized duplex is preferentially formed compared to the first hybridized duplex in the absence of the reaction-inducing agent, provided the destabilizing agent being bound to the probe, i.e., to the reaction site. Preferably, with the destabilizing agent bound to the probe, the melting temperature of the second hybridized duplex is at least about 1° C. higher, more preferably at least about 5° C. higher than that of the first hybridized duplex in the absence of the reaction-inducing agent. It is further preferred that the preferentiality of the hybridization be changed in favor of the first hybridized duplex by the action of the reaction-inducing agent. Preferably, the melting temperature of the first hybridized duplex is at least about 1° C. higher, more preferably at least about 5° C. higher than that of the second hybridized duplex in the presence of an excess of the reaction-inducing agent.

In preferred embodiments of the Type I and II coupling probes, both the first and second hybridized duplexes are thermodynamically stable in the absence of the reaction-inducing agent under the assay conditions including a detection temperature. Typically, the preferred Type I and II coupling probes have melting temperatures of the first and second hybridized duplexes in the absence of the reaction-inducing agent higher than the detection temperature, preferably 5° C. higher, and more preferably at least 10° C. higher than the detection temperature.

In preferred embodiments of the Type II(-) coupling probe, the second hybridized duplex is thermodynamically stable but the first hybridized duplex with the destabilizing agent bound thereto is unstable or less stable in the absence of the reaction-inducing agent under the assay conditions including a detection temperature. Typically, the preferred Type II(-) coupling probe has the melting temperature of the second hybridized duplex in the absence of the reaction-inducing agent higher than the detection temperature, preferably at least about 5° C. higher, more preferably at least 10° C. higher than the detection temperature.

In all types of the coupling probes described above, it is preferred to control the distance between the destabilizing agent and the nucleic acid sequence to which the destabilizing agent is linked, by changing the length of the coupling element. Use of a shorter coupling element would be preferred because steric hindrance (or other types of hindrance) gets stronger in shorter distance. Use of a larger destabilizing agent would induce stronger destabilization. However, too short distance or too large size may prohibit or suppress the activity of the reaction-inducing agent. Therefore, such parameters must be selected appropriately to maximize the destabilization effect as well as the activity of the reaction-inducing agent.

The Type I coupling probes are useful for detecting reaction-inducing agents having specific activities for conjugating (typically covalently coupling) a reaction site of interest to a destabilizing agent. As should be apparent, the invention is flexible and not limited to use of any particular reaction site. Examples of the reaction sites include those specific to various ligases such as polynucleotide ligases, aminoacyl tRNA ligases, biotin protein ligases, etc. Identity of the reaction site varies depending on the reaction-inducing agent. For instance, the reaction site will be a nucleotide sequence for a polynucleotide ligase, an amino acid for an aminoacyl tRNA ligase, and a biotin for a biotin protein ligase.

The Type II coupling probes are useful for detecting reaction-inducing agents having specific activities for converting a reaction site to a conjugation site. As should be apparent, the invention is flexible and not limited to use of any particular reaction site. Examples of the reaction sites include those specific to various transferases such as kinases, DNA polymnerases, acetyl-CoA transferases, etc. Identity of the reaction site varies depending on the reaction-inducing agent. For instance, the reaction site will be an amino acid sequence for a protein kinase, a nucleotide sequence for a DNA polymerase, and a fatty acid anion for an acetyl-CoA transferase.

The Type II(-) coupling probes are useful for detecting reaction-inducing agents having specific activities for converting a reaction site that already is a conjugation site to a non-conjugatable site. As should be apparent again, the invention is flexible and not limited to use of any particular reaction sites. One important example of the reaction site that can be used as a recognition site of the Type II(-) coupling probe is a phosphatase specific reaction site. Examples of the phosphatase specific reaction sites include, but not limited to, those specific to phosphoprotein phosphatases such as protein tyrosine phosphatases, dual specificity phosphatases and protein Ser/Thr phosphatases; phospholipids phosphatases such as phosphatidylinositol-3,4-biphosphate 4-phosphatase, phosphatidylinositol-3,4,5-triphosphate 3-phosphate, SH2 domain-containing inositol phosphatase (SHIP) and membrane-associated phospholipid phosphatase; and polynucleotide phosphatases such as polynucleotide 3′-phosphate and polynucleotide 5′-phosphatase.

Below, more specific examples are given for kinase-specific reaction sites that can be adapted to the Type II coupling probe, because detection of the kinase activity is very important in development of new drugs and diagnostics.

In general, kinase recognizes a reaction site typically having a hydroxyl group, and specifically phosphorylates the reaction site in the presence of ATP (adenosine triphosphate). For example, protein-kinase recognizes a specific amino acid sequence or a specific amino acid residue and attaches a phosphate group to a specific amino acid. By the action of the kinase, the reaction site in the Type II coupling probe is converted to a conjugation site having an additional phosphate group. It is noted that the reaction site included in the phosphatase-specific Type II(-) coupling probe is already a conjugation site having a phosphate group that will be de-phosphorylated by the action of the phosphatase. Such a phosphatase-induced reaction is exactly a reverse process of the kinase-induced reaction described above. Therefore, representative schemes described below for conjugating different types of destabilizing agents to the conjugation site included in the kinase-specific Type II coupling probe can generally be used to conjugate the destabilizing agent to the reaction site of the Type II(-) coupling probe.

First, if there exists a receptor agent that can specifically bind to the conjugation site including the phosphate group, it may be used as a destabilizing agent in a similar manner as in the affinity probe. For instance, anti-phosphotyrosine peptide antibody can be used as a destabilizing agent for Type II coupling probes having tyrosine kinase (TK)-specific reaction sites (or for Type II(-) coupling probes having protein tyrosine phosphatase-specific reaction sites), and similarly anti-phosphoserine or anti-phosphothreonine peptide antibody can be used as a destabilizing agent for serine-threonine kinase (STK)-specific Type II coupling probes (or for protein Ser/Thr phosphatase-specific Type II(-) coupling probes). In in vivo or in situ assays, such receptor agent, i.e., the destabilizing agent may be a receptor molecule naturally present in the interior or exterior of cells of interest, or it may be a receptor molecule introduced intentionally, including those recombinantly expressed inside the cell. For other instances, the following embodiments will be very useful to covalently link a destabilizing agent to the conjugation site having the phosphate group.

In another embodiment for coupling a destabilizing agent to the conjugation site including the phosphate group, a modified ATP having a reaction group that can be used to induce the coupling reaction, may be used in replacement of ATP. For example, use of adenosine 5′-O-(3-thiotriphosphate) (ATPγS) that has a sulfur atom instead of an oxygen atom in the γ-phosphate group, will lead to attachment of a thiophosphate group to the reaction site in the presence of a kinase. The thiophosphate group is known to react spontaneously with an iodoacetyl group to form a covalent linkage (Jeong, S. and Nikiforov, T. T. (1999)). Therefore, a destabilizing agent having at least one iodoacetyl group may be used in this embodiment. Alternatively, a destabilizing agent having at least one thiol group may be used. A disulfide linkage may be formed between the thiophosphate group and the thiol group in the destabilizing agent by using a coupling reagent such as BMB (1,4-bis-maleimidobutane), BMH (bis-maleimidohexane), and HBVS (1,6-hexane-bis-vinylsulfone) (See, e.g., Pierce 2001-2002 Catalog, pp. 294-331). Preferably, a protein or a protein complex having at least one iodoacetyl group or thiol group may be used as a destabilizing agent. A very useful example is streptavidin or its derivative bound to biotin in which biotin has at least one iodoacetyl group (Jeong, S. and Nikiforov, T. T. (1999)) or thiol group in the location that does not interfere with the binding of streptavidin or its derivative. Chemistry for modifying biotin is well known in the art.

In a further embodiment, the destabilizing agent that can be used for the conjugation (or reaction) site of the Type II or II(-) coupling probe having the phosphate group may be nanoparticles, microparticles, beads or membranes made of synthetic polymers or the like that contain dicataionic, tricataionic or polycatalonic metal ions, e.g., polystyrene beads or nanoparticles containing Fe³⁺, Ga³⁺ or Ru²⁺. Such particles or materials are disclosed to bind to phosphate containing moieties, e.g., the conjugation site of the kinase-specific Type II coupling probe or the reaction site of the phosphatase-specific Type II(-) probe of the present invention. See e.g., Sportsman, J. et al. PCT Pub. No. WO 00/75167 A2; Huang, W. et al. EP Pub. No. EP1156329 A2; and Nikiforov, T. T. U.S. Pat. No. 6,287,774 B1.

Preferred kinase-specific reaction sites are those that are specifically phosphorylated by a kinase associated with a human disease. For instance, various protein kinases associated with human diseases and their recognition sites are known in the art as disclosed in e.g., Pearson R. B. and Kemp B. E. (1991), “Methods in Enzyrnology”, vol. 200, pp. 62-81; Bachem catalog (2002), pp. 749-758; New England Biolabs catalog (2000), p. 162; and the WEB page, “The Protein Kinase Resources” at http://pkr.sdsc.edu/htmlidisease.shtml. Some important examples of the protein kinase-specific recognition site that can be used as the reaction site in the Type II coupling probe are listed in Table 3.

TABLE 3
Examples of protein kinases and their recognition sites⁽¹⁾

| | Recognition site⁽²⁾ | |
|---|---|---|
| Protein kinase | (SEQ ID NO) | Protein substrate |
| camp-dependent | YLRRASLAQLT (86) | pyruvate kinase |
| protein kinase | FRRLSIST (87) | phosphorylase kinase, α chain |
| (PKA, cAPK) | AGARRKASGPP (88) | histone H1, bovine |
| | GRGLSLSR (89) | human myelin basic protein |
| Casein kinase I | RTLSVSSLPGL (90) | glycogen synthase, rabbit muscle |
| (CKI, CK-1) | DIGSESTEDQ (91) | αₛ₁-casein |
| Casein kinase II | ADSESEDEED (92) | PKA regulatory subunit, R_{II} |
| (CKII, CK-2) | LESEEEGVPST (93) | p34^{cdc2}, human |
| | EDNSEDEISNL (94) | acetyl-CoA carboxylase |
| glycogen synthase | SVPPSPSLS (95) | glycogen synthase, human (site 3b) |
| kinase 3 (GSK-3) | SVPPSPSLS (96) | glycogen synthase, human (site 3a) |
| cdc2 protein kinase | PAKTPVK (97) | histone H1, calf thymus |
| | HSTPPKKKKRK (98) | large T antigen |
| calmodulin- | NYLRRRLSDSN (99) | synapsin (site 1) |
| dependent protein | KMARVFSVLR (100) | calcineurin |
| kinase II (CaMK II) | | |
| Insulin receptor | RRLIEDAEYAARG (101) | glycogen synthase |
| (INSR) | | |
| mitogen-activated | PLSP (102) | c-Jun |
| protein kinase | PSSP (103) | cyclin B |
| (MAPK, Erk) | VLSP (104) | Elk-1 |
| | KRELVEPLTPSGEAPNQ | epidermal growth factor receptor |
| | ALLR (105) | |
| CGMP-dependent | GKKRKRSRKES (106) | histone H2B |
| protein kinase | FRRLSIST (107) | phosphorylase kinase (α chain) |
| (cGPK) | RKRSRAE (108) | synthetic sequence |
| phosphorylase | DQEKRKQISVRG (109) | phosphorylase glycogen synthase |
| kinase (PhK) | PLSRTLSVSS (110) | synthetic sequence |
| protein kinase C | HEGTHSTKR (111) | fibrinogen |
| (PKC) | PLSRTLSVSS (112) | glycogen synthase |
| | QKRPSQRSKYL (113) | myelin basic protein |
| | PLSRTLSVAAKK (114) | glycogen synthase |
| | LKFSKKF (115) | synthetic sequence |
| | RKRTLRRL (116) | synthetic sequence |
| p34 cdc2 protein | AKAQHATPPKKKRKVE | simian virus 40 large T antigen |
| kinase | DPKDF (117) | |
| meiosis-activated | APRTPGGRR (118) | synthetic sequence |
| myelin basic protein | | |
| kinase (p44 mpk) | | |
| smooth muscle | KKRARTSNVFA (119) | myosin light chains (11-23) |
| myosin light chain | | |
| kinase | | |
| epidermal growth | RENAEYLRVAP (120) | autophosphorylation |
| factor receptor | AEPDYGALYE (121) | phospholipase C-γ |
| kinase (EGF-RK) | | |
| protein tyrosine | IYGEF (122) | synthetic sequence |
| kinase pp60c-src | | |
| (PTK) | | |

⁽¹⁾References: “Methods in Enzymology”, vol. 200, pp. 62-81; Bachem catalog (2002), pp. 749-758; and New England Biolabs catalog (2000), p. 162.
⁽²⁾Underlined characters denote phosphorylation sites and italic characters denote preexisting phosphorylated serine. Numbers in parentheses are SEQ ID NOs.

As discussed, each type of the probes described above is designed to undergo a readily detectable conformational change from one conformation to the other conformation depending on the presence, absence, or a reduced amount of one or more desired target agents. The conformational changes incorporated in the probes of the invention are assisted by changes in the hybridization of the object and complement nucleic acid sequences. Accordingly, nearly all the detectable labels and methods for using the labels known in the art for the nucleic acid hybridization assays (See e.g., Dattagupta, N. U.S. Pat. No. 4,968,602; Tyagi, S. et al. U.S. Pat. No. 5,925,517; Lucas, J. N. et al. U.S. Pat. No. 6,027,879) may be adapted to use in the probes of the invention. In general, at least one detectable label may be conjugated to the probes of the invention. The conjugation of the label moieties to any location in the probe must be stable under the conditions of the assay. Conjugation may be covalent or non-covalent, although covalent conjugation is preferred in most embodiments. Examples of non-covalent conjugation include, without limitation, ionic bonds, intercalation, and hydrophilic and hydrophobic interactions. Important examples will be given below for incorporating the detectable labels.

The probes according to the invention may have at least one non-interactive label conjugated to the probe, typically via a covalent linkage. Examples of non-interactive labels include fluorescers, luminescers such as chemiluminescers, radioluminescers, bioluminescers and electrochemiluminescers, radioisotopes, enzymes such as alkaline phosphatase and horseradish peroxidase, antibodies, antigens, electrochemical labels and combinations thereof. Light-generating labels such as fluorescers and luminescers may be most conveniently used. Biochemical labels including enzymes, antibodies, antigens and combinations thereof commonly known in the art for enzyme assays and ELISA that include signal amplification strategies may be very useful when detecting small amounts of target agents.

Other suitable probe labels have been disclosed. See e.g., Dattagupta, N. U.S. Pat. No. 4,968,602; Tyagi, S. et al. U.S. Pat. No. 5,925,517; and Lucas, J. N. et al. U.S. Pat. No. 6,027,879. Various probe types can be designed to suit intended use including probes with interactive labels such as a FRET (fluorescence resonance energy transfer) pair and an enzyme donor-acceptor pair, or non-interactive labels such as fluorescers, luminescers, antigens, antibodies, enzymes, and electrochemical labels. The probes of the invention are easy to design regarding optimization of the probe for certain assay conditions needed because the properties of the nucleic acid hybrids can be readily predicted based on the commonly known calculation methods or the empirical data.

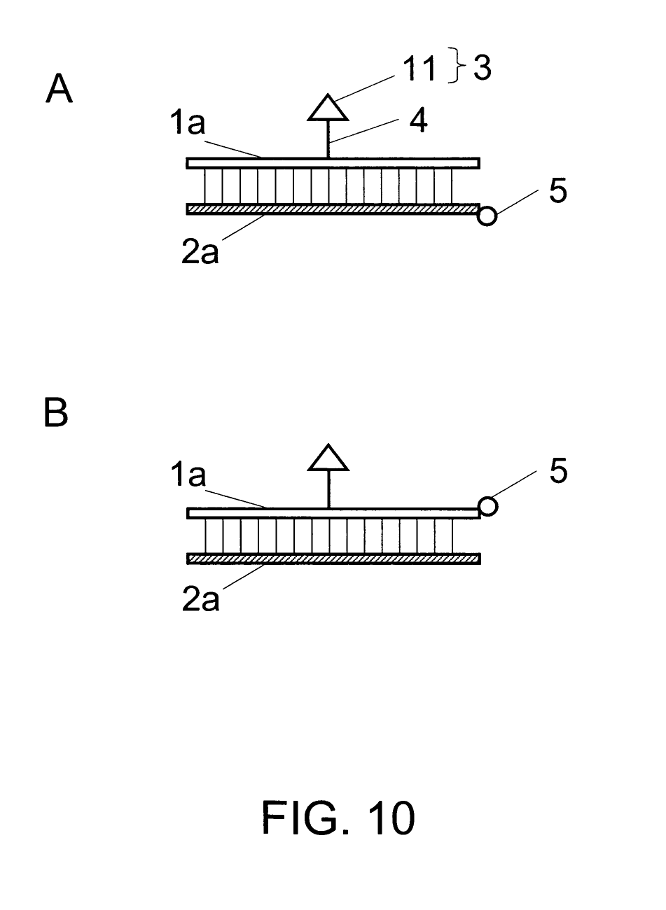

FIG. 10 illustrates a non-competitive version of a probe having at least one non-interactive label 5. An affinity probe is depicted as a particular example. Preferably, the probe is a bimolecular probe consisting of a first molecule comprising the first object sequence 1a and a second molecule comprising the first complement sequence 2a. At least one non-interactive label may be conjugated to any location in at least one of the first and second molecules, except for a location that interferes with the interaction of the recognition element 3 with the target agent (i.e., the receptor agent or the reaction-inducing agent depending on the probe format).

FIG. 11 illustrates immobilized bimolecular probes of the non-competitive version having at least one non-interactive label 5. An affinity probe is depicted as a particular example. One of the first and second molecules may be immobilized to a solid support 12 via a linker 13. Typically, the linker is a molecule having an alkyl chain or other moieties that do not interfere with the operation of the probe. As shown in FIG. 11, it is preferred that the non-interactive label be conjugated to the first or second molecule that is not immobilized.

More particular linkers suitable for use with the invention have a length of about 5 to about 100 atoms, more preferably about 5 to about 50 atoms.

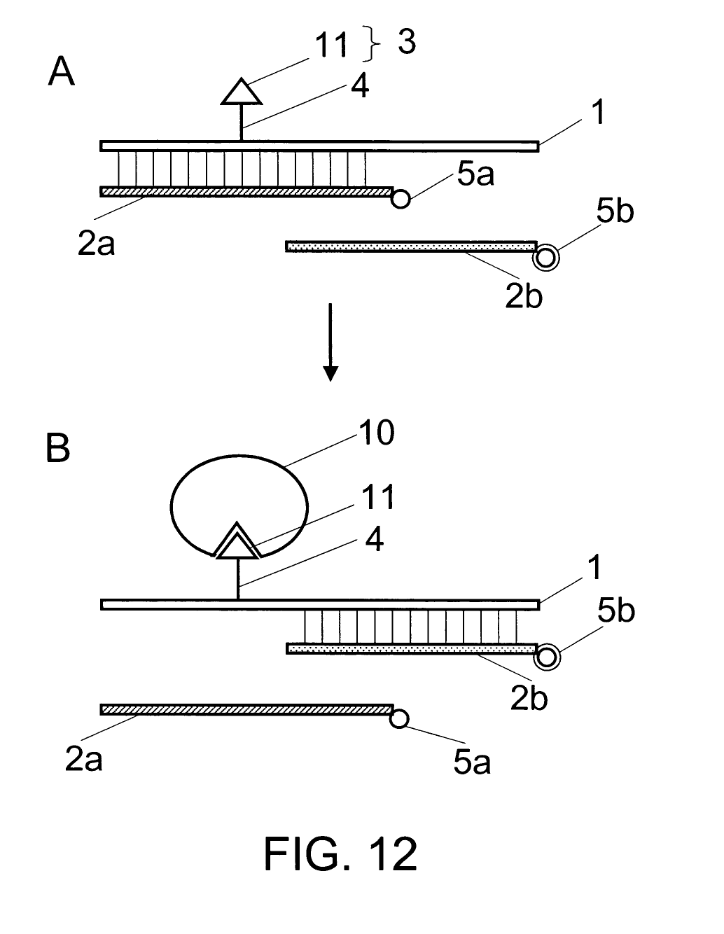

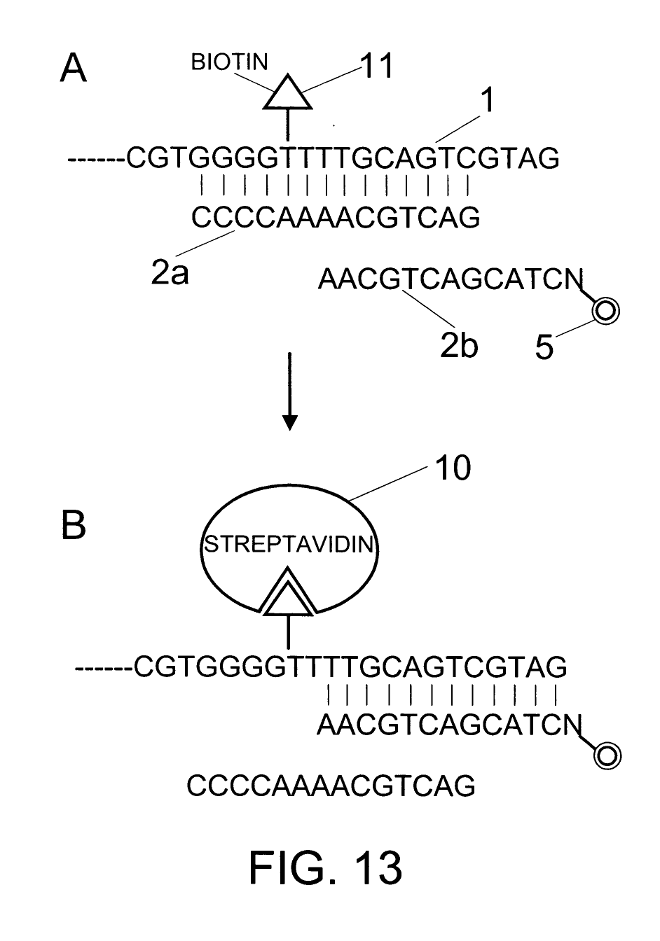

FIG. 12 illustrates a competitive version of a probe having at least one non-interactive label 5a, 5b. An affinity probe is depicted as a particular example. Preferably, the probe is a trimolecular probe consisting of a first molecule comprising the object sequence 1 consisting of the first and second object sequences, a second molecule comprising the first complement sequence 2a, and a third molecule comprising the second complement sequence 2b as shown in FIG. 13. At least one non-interactive label may be conjugated to any location in at least one of the first, second, and third molecules, except for a location that interferes with the interaction of the recognition element 3 with the target agent. In preferred embodiments, at least one non-interactive label is conjugated to at least one of the second and third molecules, wherein if both molecules are labeled, each molecule has a different label. Conjugation of different labels to the second and third molecules enables to distinguish formation of the first and second hybridized duplexes.

FIG. 13 shows an example of a competitive affinity probe with a non-interactive label used in Example 4. Biotin is used as a probe ligand to detect a receptor agent, streptavidin. A fluorescent label, fluorescein is conjugated to the second object sequence 2b. According to the preferred embodiments, the probe ligand is conjugated to the first object sequence in the region not included in the overlapping region. Further, the first complement sequence is selected to be longer than the second complement sequence. This enables that the first complement sequence hybridize preferentially over the second complement sequence in the absence of the receptor agent (FIG. 13A). Preferentiality of the hybridization changes in favor of the second complement sequence in the presence of the receptor agent (FIG. 13B) according to the preferred embodiments.

FIG. 14 illustrates immobilized trimolecular probes of the competitive version having at least one non-interactive label. An affinity probe is depicted as a particular example. As shown in FIG. 14, any one of the first, second, and third molecules may be immobilized to a solid support 12 by a linker 13. If the first molecule containing the object sequence 1 is immobilized (FIG. 14A), it is preferred to conjugate the at least one non-interactive label to at least one of the second and third molecules containing the first and second complement sequences 2a and 2b, respectively. In this case, if both of the second and third molecules are labeled, each molecule has a different label. If the second or the third molecule is immobilized as illustrated in FIGS. 14B and C, respectively, it is preferred that the at least one non-interactive label be conjugated to the first molecule containing the object sequence.

FIG. 15 shows an example of an immobilized competitive affinity probe with a non-interactive label used in Example 9. Again, biotin is used as a probe ligand to detect streptavidin, but it is conjugated to the first complement sequence 2a. The non-interactive label conjugated to the second complement sequence 2b is fluorescein. The object sequence is immobilized to a support 12 at its 5′ terminus using a linker 13. According to the preferred embodiments, the first complement sequence is selected to be longer than the second complement sequence to make the first complement sequence hybridize preferentially over the second complement sequence in the absence of the receptor agent (FIG. 15A). Preferentiality of the hybridization changes in favor of the second complement sequence in the presence of the receptor agent (FIG. 15B) according to the preferred embodiments.

In some embodiments, the competitive probe having at least one non-interactive label may be a bimolecular probe consisting of a first molecule comprising the object sequence and one of the first and second complement sequence, and a second molecule comprising the remaining complement sequence. The two sequences contained in the first molecule are covalently linked by a loop moiety. A preferred loop moiety may be any divalent or multivalent chemical moiety having a sufficient length and flexibility so as not to interfere with the hybridization of the object and complement sequences contained in the first molecule. For example, the loop moiety may be an alkyl chain, a polypeptide chain, a nucleic acid sequence or a polymer chain such as a polyethylene glycole chain and a polyester chain. If the loop moiety is a nucleic acid sequence, about 4 to about 100 nucleotides length is preferable. The loop moiety may connect any locations of the two sequences contained in the first molecule. Preferably, the loop moiety connects any of 5′ or 3′ terminus of one sequence to any of 5′ or 3′ terminus of the other sequence. More preferably, the loop moiety connects the two sequences in a 5′ to 3′ or 3′ to 5′ direction. As discussed the loop moiety may be any divalent or multivalent chemical moiety connecting any locations in the two sequences, which does not interfere with the hybridization of the object and complement sequences. In this bimolecular probe, at least one non-interactive label is conjugated to at least one of the first and second molecule. If both molecules are labeled, each molecule has a different label. In some embodiments, one of the first and second molecules may be immobilized to a support. In such embodiments, it is preferred that the at least one non-interactive label be conjugated to the molecule that is not immobilized.

If none of the molecules are immobilized in the probes having at least one non-interactive label described above, the presence of the target agent can be ascertained by detecting the characteristic signal from the label associated with hybridized or unhybridized molecules after a separation process. The separation process may be performed by electrophoresis under non-denaturing conditions as was done in some of the examples. It is desired to detect the characteristic signal as a function of the size to distinguish different species. In the immobilized probes, the presence of the target agent can be ascertained by detecting the characteristic signal from the label after washing out the unbound molecules.

Another type of detectable label that can be easily adapted to the probe is an intercalating dye such as ethidium bromide and SYBR® Green (Molecular Probes, OR, USA), which binds preferentially to double-stranded nucleic acid over single-stranded nucleic acid. When intercalating dyes are used as the detectable labels, the probe in the non-competitive version may comprise a first molecule containing the first object sequence and a second molecule comprising the first complement sequence, and one of the first and second molecules may be immobilized to a support. In some embodiments using the intercalating dyes, the probe in the non-competitive version may comprise a molecule containing both the first object and first complement sequences linked by a loop moiety, and it may also be immobilized to a support by a linker. If the probes are those in the competitive version, it is preferred that the object sequence, the first complement sequence, and the second complement sequence be contained in different molecules. Otherwise, formation of the first and second hybridized duplexes may not be distinguishable with the intercalating dye. If the probe in the competitive version is to be immobilized, it is preferred that one of the first and second complement sequences be immobilized to a support by a linker.

In embodiments in which use of intercalating dyes is desirable as the detectable label and if the probe is not immobilized, the presence of the target agent can be ascertained by detecting the characteristic signal from the label associated with the hybridized duplex(es) after a separation process using for example electrophoresis under non-denaturing conditions. As in the probes with at least one non-interactive label, it is also desired to detect the characteristic signal as a function of the size to distinguish different species. In the immobilized probes, the presence of the target agent can be ascertained by detecting the characteristic signal from the label after washing out the unbound molecules.

Interactive label pairs well known in the art in the nucleic acid hybridization assays are very useful to use as the detectable labels in the probes of this invention. Interactive label pairs useful for the probes of this invention must have a property for generating a characteristic signal, which depends on the distance between the two label moieties. As discussed, the probes according to the invention undergo a conformational change depending on the presence or absence of the target agent. The conformational change embedded in the probe is a result of hybridization and dissociation of the object and complement sequences. Therefore, nearly any interactive label pairs having a distance-dependent signal-generating property can be used by appropriately conjugating the two label moieties to the probe of the invention. Examples of useful interactive label pairs are disclosed in U.S. Pat. No. 5,925,517 regarding detection of target nucleic acid sequences. The most preferred interactive label pair to be conjugated to the probe of the invention is a pair of a fluorescer and a quencher or a pair of a luminescer and a quencher whose mode of interaction is fluorescence resonance energy transfer (FRET) or the like such as direct fluorescence energy transfer, although other types of interactive label pairs such as an enzyme and its cofactor or a pair of fragments or subunits of an enzyme including the enzyme donor and acceptor pair used in the well known enzyme complementation assays (See e.g., Comoglio, S. and Celada, F. (1976); Gonnelli, M. et al. (1981); and Henderson, D. R. U.S. Pat. No. 4,708,929) may also be used. The enzyme label pairs are particularly useful when amplification of the probe transducer signal is necessary to detect very small amounts of target agents. Examples of the luminescer include chemiluminescers, radioluminescers, electrochemical luminescers, and bioluminescers. When a FRET label pair such as a fluorescer and quencher pair or a luminescer and quencher pair is used as the detectable label, the characteristic signal is light generated from the fluorescer or the luminescer whose intensity, lifetime, or polarization changes depending on the interaction between the two label moieties. For some instances, the wavelength of the fluorescence from the probe may change upon interaction of the two label moieties. This is particularly the cases wherein the quencher moiety is also a fluorescer, preferably having a longer emission wavelength than that of the fluorescer moity and capable of acting as an energy transfer acceptor with respect to the fluorescer moity. In such embodiments, the change of the fluorescence wavelength in addition to, or in replacement of, the intensity, lifetime or polarization change may be taken as indicative of the interaction between the two label moieties caused by the probe conformational change. In most embodiments having at least one interactive label pair, two interactive label moieties are subject to interact in close proximity as in the hybridized conformation but do not interact in distal location as in the dissociated conformation. Below, some representative examples are described for preferred embodiments of the probes having at least one interactive label pair.

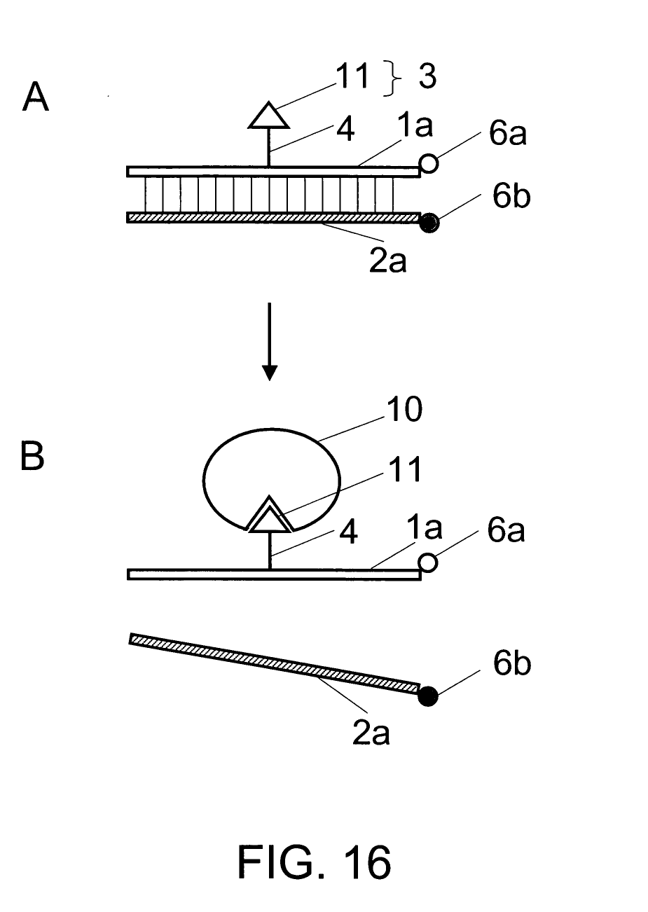

FIG. 16 schematically shows a bimolecular probe of the non-competitive version having a pair of interactive labels. An affinity probe is depicted as a particular example. In this particular embodiment, the probe comprises an interactive label pair consisting of a first label moiety 6a conjugated to the first object sequence 1a and a second label moiety 6b conjugated to the first complement sequence 2a. In the absence of the receptor agent 10, the probe is in the hybridized conformation (FIG. 16A) and the two interactive label moieties are located in close proximity, resulting in efficient interaction between the two label moieties. In the presence of the receptor agent, the probe shifts to the dissociated conformation (FIG. 16B) and the two interactive label moieties are distal to each other, prohibiting or suppressing the interaction between the two label moieties. In some embodiments, it is useful to immobilize one of the first and second molecules to a support using a linker.

FIG. 17 schematically shows a unimolecular probe of the non-competitive version having a pair of interactive labels. An affinity probe is depicted as an example. In this unimolecular probe, the first object sequence 1a and the first complement sequence 2a are covalently linked by a loop moiety. The interactive label pair consists of a first label moiety 6a conjugated to the first object sequence 1a and a second label moiety 6b conjugated to the first complement sequence 2a. In the absence of the receptor agent 10, the probe is in the hybridized conformation (FIG. 17A) and the two interactive label moieties are located in close proximity, resulting in efficient interaction between the two label moieties. In the presence of the receptor agent, the probe shifts to the dissociated conformation (FIG. 17B) and the two interactive label moieties are distal to each other, prohibiting or suppressing the interaction between the two label moieties. In some embodiments, at least one of the interactive label moieties may be conjugated to a location other than the first object or the first complement sequence, for example in the loop region, preferably in the vicinity of the first object or first complement sequence. As should be apparent, the invention is flexible and not limited to any particular locations of the interactive labels. The two interactive label pair may be located to any two different locations in the probe, as long as the desired distance-dependent interacting property described above can be attained. In some embodiments, it is useful to immobilize the unimolecular probe to a support using a linker.

FIG. 18 shows an example of a unimolecular affinity probe with an interactive label pair used in Example 3. Again, biotin conjugated to the first object (or first complement) sequence is used as a probe ligand to detect a receptor agent, streptavidin. The interactive label pair consists of a fluorescer, fluorescein and a quencher, DABCYL (4-(4′-dimethylaminophenylazo)benzoic acid), which are covalently linked to the 5′ and 3′ terminus of the unimolecular probe, respectively. According to the invention, the probe is designed to undergo a conformational change from a hybridized conformation (FIG. 18A) to a dissociated conformation (FIG. 18B) upon binding of the receptor agent, which results in increase of the fluorescence intensity from the fluorescer.

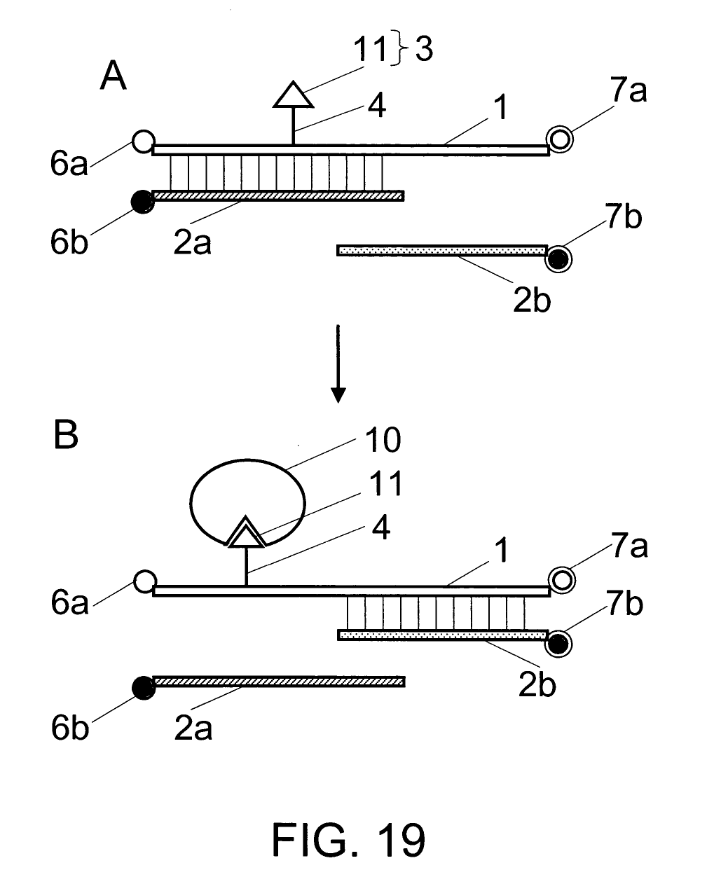

The competitive version of the probe may also be constructed by conjugating at least one interactive label pairs to at least one pair of the object and complement nucleic acid sequences. FIG. 19 schematically shows a competitive version of the probe having two different pairs of interactive labels. An affinity probe is depicted as a particular example. In this particular embodiment, a first pair of interactive labels consists of a first label moiety 6a conjugated to the first object sequence 1a and a second label moiety 6b conjugated to the first complement sequence 2a, and a second pair of interactive labels consists of a third label moiety 7a conjugated to the second object sequence 1b and a fourth label moiety 7b conjugated to the second complement sequence 2b. In the absence of the receptor agent 10, the probe is in the first hybridized conformation (FIG. 19A), and thus the first label pair is located in close proximity while the second label pair is distal to each other. This results in efficient interaction between the first label pair, but prohibiting or suppressing the interaction between the second label pair. In the presence of the receptor agent, the probe shifts to the second hybridized conformation (FIG. 19B), and thus the first label pair becomes distal to each other while the second label pair becomes located in close proximity. Therefore, interaction between each label pair becomes reversed. As should be apparent, the interactive label pair(s) may be conjugated to only one pair of the object and complement sequences, or to both pairs of the object and complement sequences. If both pairs of the object and complement sequences are labeled, it is preferred that each pair of the object and complement sequences have a different interactive label pair. For a particular instance, the first and third label moieties 6a and 7a may be the same moiety conjugated at a suitable position (e.g., around the middle of the object sequence) that can interacts with both the second and fourth moieties 6b and 7b that are also conjugated at suitable positions (e.g., those positions that are close proximity with respect to the label moiety conjugated to the object sequence when hybridized).

As shown in FIG. 19, the competitive version of the probe having at least one interactive label pair may be a trimolecular probe consisting of a first molecule containing the object sequence, a second molecule containing the first complement sequence, and a third molecule containing the second complement sequence. This trimolecular probe may have one of the first, second, and third molecules immobilized to a support by a linker. In other embodiments, the competitive probe may be a bimolecular probe consisting of a first molecule containing two of the object sequence, the first complement sequence, and the second complement sequence, and a second molecule containing a remaining sequence. The two sequences contained in the first molecule are covalently linked by a loop moiety. This bimolecular probe may have one of the first and second molecules immobilized to a support by a linker. In still other embodiments, the competitive probe may be a unimolecular probe having the object sequence, the first complement sequence, and the second complement sequence being covalently linked by at least one loop moiety. This unimolecular probe may also be immobilized to a support by a linker. In the bimolecular and the unimolecular probe described above, at least one of the interactive label moieties may be conjugated to a location other than the object, the first complement, or the second complement sequence, for example in the loop region, preferably in the vicinity of the object or complement sequences. As discussed the loop moiety may be any divalent or multivalent chemical moiety connecting any locations in the two sequences, which does not interfere with the hybridization of the object and complement sequences.

In addition to probe embodiments that feature interactive label pairs described above, there are different ways to construct the probes having interactive label pairs within the scope of this invention. One more particular example will be described below as an example.

In the embodiments described above, each pair of interactive label moieties is conjugated to a pair of object and complement sequences, i.e., one to the first or second object sequence and the other to the corresponding complement sequence. In the embodiments to be described, both interactive label moieties in each pair are conjugated to one of the object or complement sequence by using additional arm sequences.

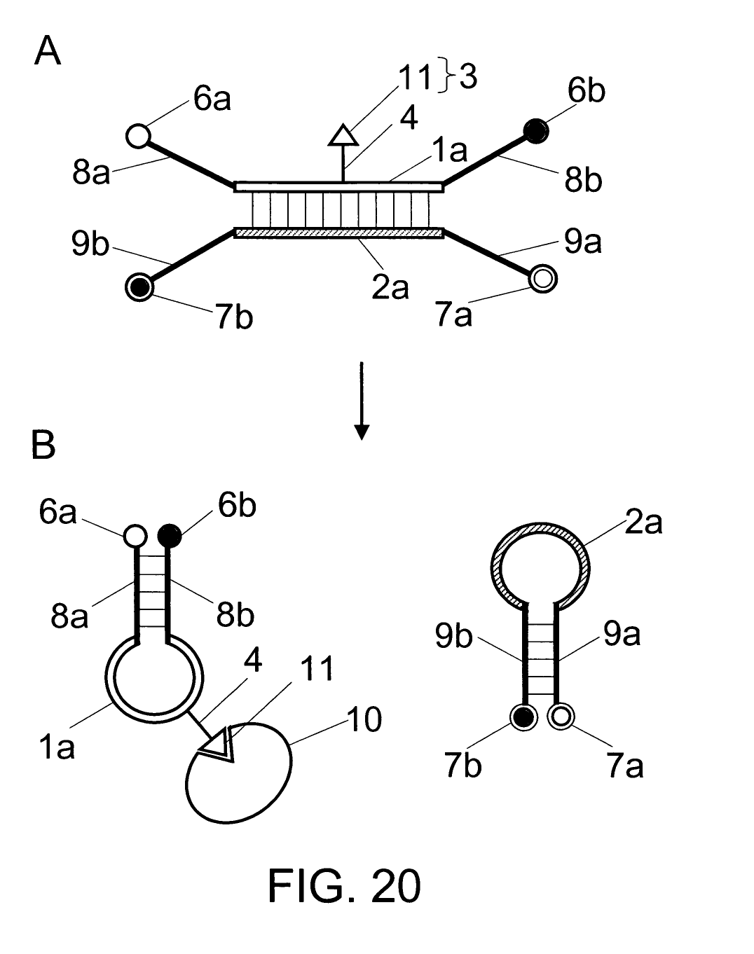

FIG. 20 illustrates an example for a non-competitive version of the probe. An affinity probe having two pairs of interactive labels is depicted as a particular example. In this particular embodiment, the probe further comprises two pairs of nucleic acid arm sequences consisting of first and second arm pairs. The first arm pair consists of a first 5′ arm sequence 8a covalently linked to 5′ terminus of the first object sequence 1a and a first 3′ arm sequence 8b covalently linked to 3′ terminus of the first object sequence 1a. The second arm pair consists of a second 5′ arm sequence 9a covalently linked to 5′ terminus of the first complement sequence 2a and a second 3′ arm sequence 9b covalently linked to 3′ terminus of the first complement sequence 2a. Each pair of the arm sequences are complementary to each other, and thus the first and second arm pairs form a first and a second stem duplex having about 3 to about 35 complementary base pairs, respectively, when the probe is in the dissociated conformation (FIG. 20B). When the probe is in the hybridized conformation (FIG. 20A), the first and second stem duplexes are subject to dissociate. The probe further comprises two interactive label pairs, a first label pair comprising a first label moiety 6a conjugated to the first 5′ arm sequence and a second label moiety 6b conjugated to the first 3′ arm sequence, and a second label pair comprising a third label moiety 7a conjugated to the second 5′ arm sequence and a fourth label moiety 7b conjugated to the second 3′ arm sequence. The first and second label pairs are subject to interact when the first and second stem duplexes are formed, respectively.

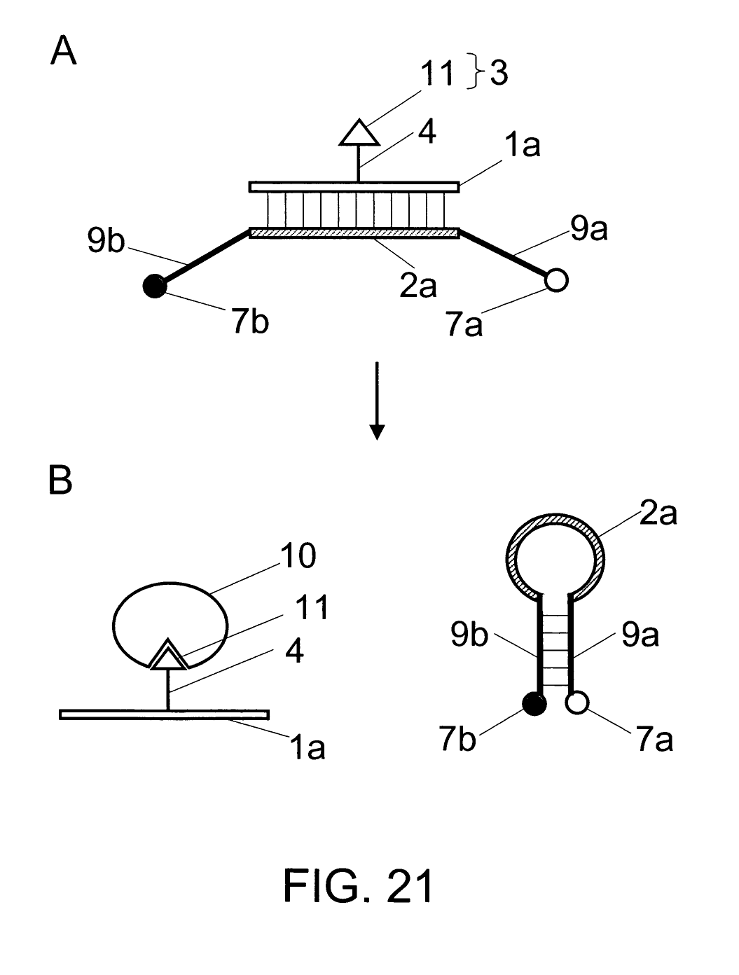

In the absence of the receptor agent 10, the probe is in favor of the hybridized conformation (FIG. 20A), and both stem duplexes are dissociated. Therefore, each interactive label pair is located distal to each other, prohibiting or suppressing the interaction between the two label moieties in each pair. In the presence of the receptor agent, the probe shifts to the dissociated conformation (FIG. 20B), and the stem duplexes are formed. Therefore, each interactive label pair is located in close proximity, the interaction between the two label moieties being effective. As should be apparent, the interactive label pair(s) may be conjugated to only one of the first object sequence or the first complement sequence (see e.g., FIG. 21), or to both of the first object and the first complement sequence (see e.g., FIG. 20). The probe may be a bimolecular probe consisting of a first molecule containing the first object sequence and a second molecule containing the first complement sequence, or it may be a unimolecular probe having the first object sequence and the first complement sequence being covalently linked by a loop moiety. The probe may be an immobilized probe tethered to a support by a linker. If the immobilized probe is a bimolecular probe, one of the first and second molecules is immobilized. For some instances, at least one of the interactive label moieties may be conjugated to a location other than the arm sequences, for example in the loop region or in the first object or first complement sequence, preferably in the vicinity of the arm sequences.

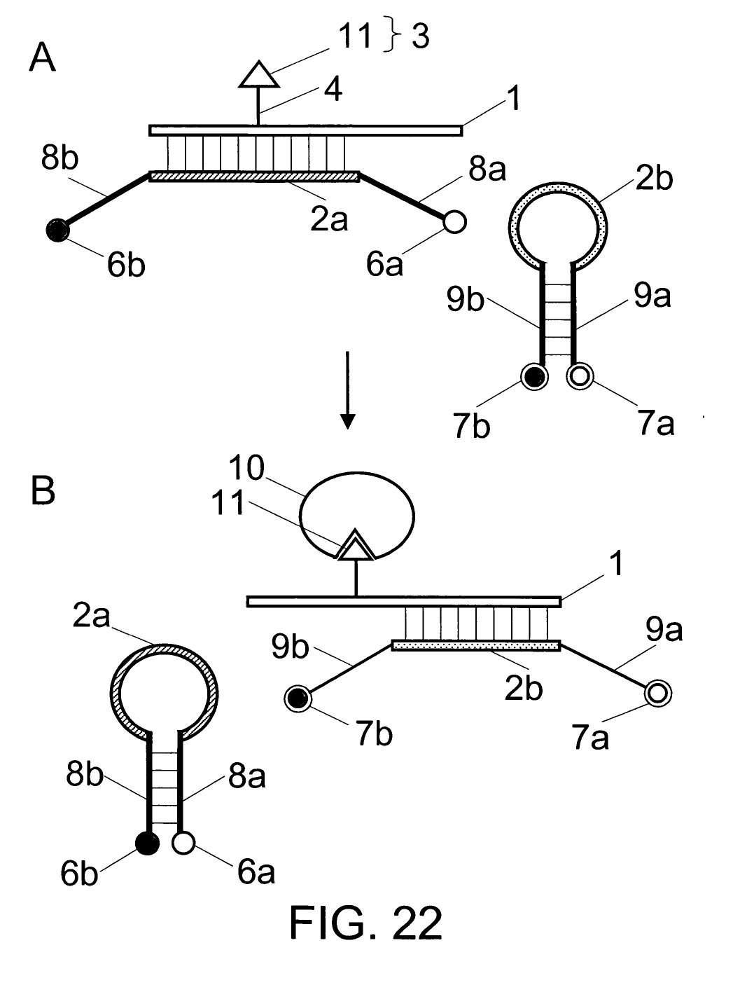

FIG. 22 illustrates an example for a competitive version of the probe having two competing pairs of the object and complement sequences. An affinity probe having two pairs of interactive labels is depicted as a particular example. In this particular embodiment, the probe further comprises two pairs of nucleic acid arm sequences consisting of first and second arm pairs. The first arm pair consists of a first 5′ arm sequence 8a covalently linked to 5′ terminus of the first complement sequence 2a and a first 3′ arm sequence 8b covalently linked to 3′ terminus of the first complement sequence 2a. The second arm pair consists of a second 5′ arm sequence 9a covalently linked to 5′ terminus of the second complement sequence 2b and a second 3′ arm sequence 9b covalently linked to 3′ terminus of the second complement sequence 2b. Each pair of the 5′ and 3′ arm sequences is complementary to each other. The first arm pair forms a first stem duplex having about 3 to about 35 complementary base pairs when the first complement sequence is not hybridized, and the second arm pair forms a second stem duplex having about 3 to about 35 complementary base pairs when the second complement sequence is not hybridized. When the first or the second complement sequence is hybridized to the object sequence, the corresponding stem duplex is subject to dissociate. The probe further comprises two interactive label pairs, a first label pair comprising a first label moiety 6a to the first 5′ arm sequence 8a and a second label moiety 6b conjugated to the first 3′ arm sequence 8b and a second label pair comprising a third label moiety 7a conjugated to the second 5′ arm sequence 9a and a fourth label moiety 7b conjugated to the second 3′ arm sequence 9b. The first and second label pairs are subject to interact when the first and second stem duplexes are formed, respectively.

In the absence of the receptor agent 10, the probe is in the first hybridized conformation (FIG. 22A), and thus the first stem duplex is dissociated and the second stem duplex is formed. Therefore, the first label pair is located distal to each other, prohibiting or suppressing the interaction between the two label moieties in the first label pair. The second interactive label pair is located in close proximity, the interaction between the two label moieties in the second label pair being effective. In the presence of the receptor agent, the probe shifts to the second hybridized conformation (FIG. 22B), and thus the first stem duplex is formed and the second stem duplex is dissociated. In the second hybridized conformation, the first label pair interacts efficiently and the second label pair interacts sufficiently weakly or does not interact. As should be apparent, the interactive label pair(s) may be conjugated to only one of the first or second complement sequence, or to both of the first and second complement sequence. If both complement sequences are labeled, it is preferred that each complement sequence has a different interactive label pair. The probe may be a trimolecular probe consisting of a first molecule containing the object sequence, a second molecule containing the first complement sequence, and a third molecule containing the second complement sequence. The trimolecular probe may be an immobilized probe having one of the first, second, and third molecules being immobilized to a support by a linker. The probe may be a bimolecular probe consisting of a first molecule containing two of the object sequence, the first complement sequence, and the second complement sequence, and a second molecule containing a remaining sequence. The two sequences in the first molecule is covalently linked by a loop moiety. The bimolecular probe may be an immobilized probe having one of the first and second molecules being immobilized to a support by a linker. The probe may be a unimolecular probe having the object sequence, the first complement sequence, and the second complement sequence being covalently linked by at least one loop moiety. The unimolecular probe may also be an immobilized probe tethered to a support by a linker. For some instances, at least one of the interactive label moieties may be conjugated to a location other than the arm sequences, for example in the loop region or in the first object or first complement sequence, preferably in the vicinity of the arm sequences.

In most embodiments having at least one interactive label pair described above, there is usually no requirement for a one-to-one molecular correspondence between the members of the label pair. Each member may consist of more than one conjugated molecules, as long as one member or at least one molecule of one member can affect, or be affected by, at least one molecule of the other member so that at least one measurable characteristic of one member is altered by the other member in a distance-dependent manner. The characteristic signal of the interactive label pair is detectably different depending on the conformation of the probe (i.e., either the hybridized or dissociated conformation in the non-competitive version, or either the first or second hybridized conformation in the competitive version). A label moiety consisting of more than one conjugated molecules may be very useful in some embodiments because a larger characteristic signal can be generated. When using FRET label pairs such as a fluorescer and quencher pair or a luminescer and quencher pair, multiple quenchers may be coupled to a light-generating moiety. This is especially useful because quenching of the light-generating moiety by the quencher becomes very efficient and thus background signal can be effectively removed.

As discussed, the most preferred interactive label pair to be used is a FRET pair such as a fluorescer and quencher pair or a luminescer and quencher pair. In such instances, the characteristic signal may be the intensity, lifetime, or polarization of the light at a particular wavelength generated by the fluorescer or the luminescer moiety. Interaction between the two label moieties will result in decrease in intensity, decrease in lifetime, or change in polarization (or polarization lifetime) of the light generated by the fluorescer or the luminescer.

As also discussed, enzyme label pairs including the enzyme donor and acceptor pair are preferred particularly for detecting very small amounts of target agents because, e.g., the probe signal can be amplified by means of an enzyme reaction.

The probes with interactive label pairs are especially useful compared to the probes with other detectable labels such as non-interactive labels and intercalating dyes. Use of the interactive label pairs with the probes that are not immobilized allows homogeneous assays in which the separation process typically necessary for other types of labels is not required. For example, if FRET label pairs such as a fluorescer and quencher pair and a luminescer and quencher pair are used, the characteristic signal can be ascertained or measured by simply detecting the property of the light emitted from the sample without any separation process. Therefore, the probes with interactive label pairs can be used in in situ or in vivo assays. The immobilized probes having at least one interactive label pair are also advantageous, because the washing step typically necessary for other types of labels may not be required.

In some embodiments, the probes according to this invention may be formatted to include additional components needed for particular applications.

In one embodiment, one or more transit peptides or targeting sequences may be conjugated to the invention probe in order to translocate such probe to a specific subcellular organelle inside a cell such as the nucleus, mitochondria, peroxisome, chloroplast, vacuole, Golgi apparatus etc. Preferably, such transit peptides or targeting sequences are conjugated (typically via a covalent linkage) to the probe at locations that do not interfere with the intended probe function. Assays using this type of probes can be used in in vivo or in situ assays to detect target agents in subcellular organelles. Probes having interactive label pairs are generally preferred for such intracellular targeting assays, those of the unimolecular format being most preferred.

In other embodiments, one or more additional nucleic acid sequences may be conjugated to the invention probe, particularly to the nucleic acid based signal transducer. More specifically, the additional nucleic acid sequences can be conjugated to the object and/or complement sequences, preferably via covalent linkages. One of the particular usages of such probes, particularly those having non-interactive labels or intercalating dye as a detectable label, is to aid size-dependent identification of hybridized or unhybridized species. For instance, in the trimolecular competitive probe format, the first or second complement sequence can be formatted to include an additional nucleic acid sequence that is not sufficiently complementary to the corresponding portion (if exists) of the object oligonucleotide. Such embodiment will lead to the first hybridized duplex (or the first complement sequence) being sufficiently different in size from the second hybridized duplex (or the second complement sequence) in electrophoretic analysis.

In another embodiment, nearly any of the invention probes described herein can be formatted to include one or more additional nucleic acid sequences conjugated to at least one of the object and complement sequences, that are made to serve as nucleic acid replication templates. As discussed, such probes are compatible with use of standard PCR (or polymerization reaction) amplification strategies to detect a desired hybridized state. More specifically, if the probe is of the non-competitive format, either the first object sequence or the first complement sequence can be formatted to include a nucleic acid replication template. A polymerase or related enzyme can then be used to facilitate a PCR or polymerization reaction that is sensitive to the amount of the hybridized conformation. If the probe is of the competitive format, the probe needs to be formatted such that a PCR or polymerization reaction can take place for only one of the first or second hybridized conformation. This can be done in various ways. For example, if the nucleic acid replication template is to be included in the object sequence, one of the first and second complement sequences can be treated with phosphatase to remove the phosphate group in the 5′ terminal. Such treatment will lead to blocking of the PCR or polymerization reaction resulting from such treated complement sequence without the 5′ phosphate group. Alternatively, the nucleic acid replication template can be included in 5′ terminus of only one of the first and second complement sequences so that the PCR or polymerization reaction can take place when the complement sequence having the replication template is hybridized to the object sequence. As discussed, presence of such amplification products or a detectable decrease in the amount of polymerization reagents (or both), can be taken to be indicative of the presence of the target agent in the sample.

In general, preparation of the present target detection system and especially the probes disclosed herein can be accomplished by procedures disclosed herein and by recognized laboratory techniques. For example, nucleic acid molecules included in the probe, particularly in the nucleic acid based signal transducer can be prepared by commonly known techniques of solid phase synthesis (See e.g., Caruthers, M. H. (1985); Brown T. and Dorcas, J. S. (1995)), by ligation of synthetic sequences or restriction fragments, or by a combination of these techniques. Typically, other components including the recognition elements and the detectable labels can be incorporated into the probe by using in the nucleic acid synthesis, modified nucleotides (including abasic nucleotides and other nucleotide analogues) having such component(s) conjugated thereto. Alternatively, modified nucleotides with reactive groups linked thereto as an adaptor can be used in the nucleic acid synthesis. Other components such as the recognition elements, the detectable labels, the loop moieties, etc. can then be conjugated to the reactive group(s) by using commonly known chemical or biochemical coupling schemes. Such chemical or biochemical coupling may involve reactive groups such as hydroxyl, carboxyl, amino, sulfhydryl, aldehyde, phosphate, alkyl amino, hydroxy alkyl, alkyl phosphate and the like. Techniques for preparing modified nucleotides with various reactive groups are well known in the art. Typically, synthesis of the nucleic acid molecules having modified nucleotides involves the well known solid phase synthesis techniques. Preparation and/or conjugation of the recognition elements, the detectable labels and other components can be generally performed using known chemical, biochemical and biological methods. For example, amino acid sequences specific to proteases and protein kinases can be prepared by well known solid phase or other synthesis techniques (See e.g., Wunsch, E. (1971); Houghtem, R. A. (1985)). Proteins or polypeptides such as receptors, cytokines, protein hormones, antigens, enzymes and the like can be prepared by recombinant DNA techniques including recombinant DNA preparation, transformation, cell culture, and isolation and purification of the expressed proteins or polypeptides. Carbohydrates, lipids, phospholipids and other chemical or biochemical entities can be prepared by using known chemical, biochemical and biological techniques. In addition, techniques for incorporating (or conjugating) chemical or biochemical entities into nucleotides or nucleic acid molecules are well known in the art. Other suitable laboratory procedures in line with the invention have been reported. See generally Knorre, D. G. et al. in Design and targeted reactions of oligonucleotide derivatives, CRC Press, Boca Raton, Ann Arbor, London, Tokyo (1994); Travers, A. and Buckle, M. in DNA-Protein Interactions: A Practical Approach, Oxford University Press (2000); Sambrook et al. in Molecular Cloning: A Laboratory Manual (2nd ed. 1989); Ausubel et al. in Current Protocols in Molecular Biology, John Wiley & Sons, New York (1989); and Ausubel et al. in Short Protocols in Molecular Biology, John Wiley & Sons, New York (1999) for disclosure relating to these methods.

See also the Examples section (disclosing methods of making and using particular target detection systems)

Assays according to the invention for detecting at least one target agent typically comprise: a) contacting a probe according to this invention to a sample suspected to contain a target agent; and b) ascertaining any change in the level of the characteristic signal under the assay conditions including a detection temperature compared to that in the absence of the target agent. The probe will be an affinity probe according to the invention to detect a receptor agent that can specifically bind to the probe ligand included in the affinity probe used. The probe will be a cleavage probe according to the invention to detect a reaction-inducing agent that can specifically cleave the cleavage site included in the cleavage probe used. The probe will be a Type I coupling probe according to the invention to detect a reaction-inducing agent that can specifically induce a covalent or non-covalent coupling of the reaction site included in the Type I coupling probe used. The probe will be a Type II coupling probe according to the invention to detect a reaction-inducing agent that can specifically convert the reaction site included in the Type II coupling probe to the conjugation site. In another invention example, the probe is a Type II(-) coupling probe that is designed to detect a reaction-inducing agent. Typically, the reaction-inducing agent in this probe embodiment specifically converts the reaction site (conjugation site) of the Type II(-) coupling probe to the non-conjugatable site.

The affinity and reaction probes according to this invention can also be used in assays for detecting or identifying target agents other than the receptor agents and the reaction-inducing agents. Some particular examples are described below.

The affinity probes can also be used in assays for detecting at least one target ligand. These assays comprise: a) contacting an affinity probe according to this invention to a sample suspected to contain a target ligand in the presence of a receptor agent that can bind to both the probe ligand and the target ligand; and b) ascertaining any change in the level of the characteristic signal under the assay conditions including a detection temperature compared to that in the absence of the target ligand. Assays using the affinity probes also include assays for identifying the target ligand, i.e., determining whether a candidate target ligand binds to the receptor agent of interest, and assays for detecting other factors or entities including those that affect interaction of the receptor agent and the probe ligand. Such assays are typically performed in the presence of the receptor agent of interest.

The reaction probes can also be used in assays for detecting target agents other than the reaction-inducing agents. These assays typically comprise: a) contacting a reaction probe according to this invention to a sample suspected to contain a target agent in the presence of a reaction-inducing agent that can specifically interact with the reaction probe; and b) ascertaining any change in the level of the characteristic signal under the assay conditions including a detection temperature compared to that in the absence of the target agent. The target agents in these assays could be other factors or entities in the sample that are needed for intended interaction of the reaction-inducing agent with the reaction probe and/or for intended functioning of the probe. Examples of such target agents include certain factors such as cofactors, coenzymes, metal ions, secondary substrates and the like that are closely tied to the activity of the reaction-inducing agent. In the Type I, II and II(-)coupling probes, such target agents could be certain entities in the sample including those that can act as the destabilizing agent to be coupled or conjugated to the probe. The assays using the reaction probes also include assays for identifying such factors and entities, i.e., determining whether a candidate factor or entity affects interaction of the reaction-inducing agent and the probe. Such assays are typically performed in the presence of the reaction-inducing agent of interest.

The invention further provides assays for detecting inhibitors (or enhancers) that inhibit (or enhance) interaction of a target agent with the recognition element. These assays typically comprise: a) contacting a probe according to this invention to a candidate compound in the presence of the target agent; and b) ascertaining any change in the level of the characteristic signal under the assay conditions including a detection temperature compared to that in the absence of the candidate compound. Again, the probe will be an affinity probe when the target agent is a receptor agent. The probe will be a cleavage probe according to the invention when the target agent is a reaction-inducing agent having a cleavage activity. The probe will be a Type I coupling probe when the target agent is a reaction-inducing agent having a coupling activity. The probe will be a Type II or II(-) coupling probe when the target agent is a reaction-inducing agent having a modification activity. The step of contacting in the assays using the Type I, II or II(-) coupling probe includes adding a destabilizing agent, and optionally a coupling reagent in the assays using the Type II or II(-) coupling probe.

Nearly any compound or group of compounds can be screened to detect inhibitors or enhancers which block or enhance the interaction of the target agent and the recognition element in accord with this invention. Examples include, but are not limited to, cytokines, tumor suppressors, antibodies, receptors, muteins, fragments or portions of such proteins, and active RNA molecules, e.g., an antisense RNA molecule or ribozyme. A preferred compound for screening purposes is a synthetic or semi-synthetic drug (referred to sometimes as a “small molecule”). Examples include chemical compounds, peptides, mixtures of chemical compounds or peptides, and extracts of natural products. Typically, the concentration of the candidate inhibitor or enhancer that can be examined is about 0.1 to 100 μM, often lower. In typical screening, concentration of about 1 to about 10 μM may be preferred. In the case that a large number of candidate compounds need to be screened, mixtures of about 5-30 candidate inhibitors can be used in the first round of the screening.

A variety of candidate compound collections are known and can be used in accord with the present target detection system.

For example, a pool of derivatives of known inhibitors of human viral pathogens can be readily tested by the present methods. See e.g., U.S. Pat. Nos. 6,420,438; 6,329,525; 6,287,840; 6,147,188; and 6,046,190 (disclosing a variety of testable molecules and derivatives thereof). It is possible to use the invention to screen additional compounds. See Pillay et al. (1995) Rev. Med. Virol. (disclosing a variety of potential viral protease inhibitor compounds); and Wei et al. (1995) Nature, 373: 117 (disclosing indinavir, ABT-538); Ho et al. (1992) Ann. Intern. Med. 113: 111 (disclosing an anti-herpes agent). Also, derivatives of the forgoing specific compounds can be screened in accord with the invention including, but not limited to, saquinavir and derivatives thereof.

The present target detection system can be used with other compound collections including those that include modulators of one or more protein kinases. Such collection can include random peptide libraries and combinatorial chemistry-derived molecular libraries such as those made of D-, L- or D- and L-amino acids. See Lam et al., Nature 354:82-84 (1991); and Houghten et al., Nature 354:84-86 (1991). Also envisioned is use of phosphopeptide libraries (e.g., members of random and partially degenerate, directed phosphopeptide libraries). See eg., Songyang et al., Cell 72:767-778 (1993).

The invention is compatible with use of more particular libaries of kinase modulators such as those disclosed in U.S. Pat. Nos. 6,462,060; 6,420,382; and 6,462,036.

The assays described above may be qualitative or quantitative. Quantitative assays can employ measuring methods known in the art. For example, a characteristic signal of a sample may be compared to those of a target dilution series. Also, measuring may be taken as a function of time, or compared to readings of a negative and/or positive control.

As discussed in detail, the probes according to the invention may have at least one detectable labels selected from non-interactive labels, intercalating dyes, or interactive label pairs. Also, the probes according to the invention may be trimolecular, bimolecular, or unimolecular probes. Further, the probes according to the invention may be of non-competitive or competitive version. All these probes can be used in the assays described above. Specifically, if the probe has non-interactive labels or intercalating dyes as the detectable labels and it is not immobilized, the step of ascertaining includes a step of separating hybridized and unhybridized species as a function of size. If the probe having non-interactive labels or intercalating dyes is an immobilized probe, the step of ascertaining includes a step of washing to remove unbound species from the support to which the probe or its component is immobilized. If the probe has interactive-label pairs as the detectable labels, the assays can be either homogeneous or heterogeneous, although homogeneous assays are more preferred. The most preferred interactive label pair is a FRET pair or the like such as a fluorescer and quencher pair or a luminescer and quencher pair, although the aforementioned enzyme label pair is preferred when signal amplification strategy is needed to detect very small amounts of target agents.

The probes having interactive label pairs are especially useful in in situ or in vivo assays for detecting target agents or screening inhibitors or enhancers in the interior or exterior of tissues or cells without destruction of the tissues or the cells. Because a large excess of the probes can be used without the need for washing or separation and in some embodiments without generation of a large background signal, in situ or in vivo assays of this invention are particularly useful. For in situ or in vivo assays, unimolecular probes having interactive label pairs are most preferred probes, preferably the interactive label pair being a pair of a fluorescer and a quencher or a pair of a luminescer and a quencher. In in situ or in vivo assays and screens, the step of contacting may include adding to a sample containing tissues or cells to be examined, or introducing into the cells, the probe(s) to be used and other agents if necessary; for example, a candidate compound, a receptor agent, a reaction-inducing agent, a destabilizing agent, a vector encoding a receptor agent, a vector encoding an enzyme that acts as a reaction-inducing agent, etc. In in situ and in vivo assays according to this invention, the probes and other agents can be introduced into the interior of the cells via known techniques, e.g., by encapsulating the probes and other agents with liposomes or by making the cell membranes porous or permeable to the probes and other agents. Microinjection technique is particularly useful for studying a single cell, especially as a function of time depending on its growth stage or in response to various stress or induction signal.

The in situ or in vivo assays according to this invention include “vital staining”, i.e., staining specific constituents of living cells or tissues without killing them. Such assays can be used to identify (and sometimes to count) specific cell types within a tissue or a living organism or within a collection or mixture of cells. The in vivo assays according to this invention are particularly useful to locate specific target agents in the interior or exterior of various living cells or in various subcellular organelles inside living cells. The probes according to this invention can report not only the inter- or intracellular location of the target agent but also the interaction activity of the target agent and the recognition element in a correlated manner. This feature is particularly advantageous over conventional staining methods using labeled receptor or ligand molecules, because false readings can be largely avoided and more detailed information can be obtained for the target agent of interest. Particularly interesting in vivo assays are those using the probes having targeting sequences (including transit peptides) as recognition elements. Targeting sequences are known to direct intercellular translocation to different cell types or intracellular translocation to various subcellular organelles. Such translocation processes are believed to involve binding of certain molecular entities to the targeting sequences and sometimes processing such as cleavage, of the targeting sequences. Such interactions of the targeting sequences during the translocation as well as the location of the probe or the target agent inside or outside the cell can be registered and reported by using the probes according to this invention. For instance, a particular probe of the competitive format having two different FRET label pairs can be constructed according to the invention to generate different detectable signal (i.e., fluorescence signal at different wavelength) depending on the probe conformation, either the first or the second hybridized conformation. For another instance, a particular probe of the non-competitive format having a wavelength-changing FRET label pair can be constructed for the same purpose. As discussed, if the quencher moiety is another fluorescer, preferably one having a longer fluorescence wavelength than the fluorescer moiety, the fluorescence wavelength from the probe can be altered depending on the probe conformation. Use of such type of wavelength-changing probes will make it possible to independently monitor the location of the probe inside the cell and the interaction of the target agent and the probe recognition element.

The probes according to the present invention are well suited for use in a variety of high throughput assays and screens. For instance, the probes of this invention can be readily adapted to use in microfluidic Lab-on-a-chips including those manufactured by Caliper Technologies Corp. and Agilent Technologies Inc. Such Lab-on-a-chip devices typically embody electrophoretic separation and optical (either absorption or emission) detection scheme. See e.g., Kennedy, C. B. U.S. Pat. No. 5,885,470; Parce, J. W. et al. U.S. Pat. No. 5,885,470; and Parce, J. W. et al. U.S. Pat. No. 5,942,443. Therefore, the probes of this invention having light generating or absorbing labels, either non-interactive or interactive, can be most preferably used with such devices to report or register the presence or absence of the target agent or its activity in a sample. The conformational change embodied in the invention probe resulting from the interaction of the target agent and the probe usually accompanies change in the electrophoretic property that can be readily detected in the Lab-on-a-chip devices by means of the light-generating or absorbing labels.

Other examples of using the invention probes in high throughput assays and screens include, but not limited to, biochips such as protein chips including immuno-assay microchips (See e.g., Cohen, C. et al. (1999); Sato, K. et al. (2000)) which typically incorporate immobilized probe molecules. Nearly any type of the immobilized probes according to the invention can be readily adapted to use in such high throughput biochip devices and assays. The invention probes, both immobilized and un-immobilized formats are also well suited to use in microtiter well based high throughput devices and assays including ELISA (See e.g., Bosworth, N. and Towers, P. (1989); Pope, A. J. et al. (1999)).

The invention also provides kits that can be used to perform the assays described above. The kit according to the invention typically comprises at least one probe according to the invention and instructions for performing the assays. The assays may be for detecting at least one target agent or target ligand, or detecting inhibitors or enhancers for inhibiting or enhancing interaction of at least one target agent with the recognition elements. The kits may include one or more reagents selected from salts, buffers, nuclease inhibitors, substrates for enzymes or enzyme-coupled labels, receptor agents, reaction-inducing agents, vectors encoding receptor agents, vectors encoding enzymes acting as reaction-inducing agents, and coupling reagents. For in situ or in vivo assays and screens, the kits may include a component necessary to introduce a probe and other agents into a cell, selected from permeabilizing agents or liposome precursors.

Assay kits according to this invention generally include at least one target detection system of the invention which system can include at least one probe and instructions for performing one or more detection strategies. Kits may also include assay reagents, e.g., salts, buffers, nuclease inhibitors, restriction enzymes and denaturants. Kits may include a target or model target for a positive control test, and a target-less “sample” for a negative control test.

Kits suitable for use in the field (e.g., environmental waste site) use are also contemplated. In this example of the invention, such kits will preferably include a target detection system configured for using probes that are often operably linked to a solid support e.g., beads, wells or a dipstick. Multiple probes may be included, including a positive control probe that will hybridize to a component of a known target in a sample.

“Pre-probes” according to the invention and kits comprising those pre-probes can be used to construct nearly all of the probes described herein by using conventional synthetic techniques. The pre-probes typically include one or two pairs of substantially complementary nucleic acid sequences that can function as the aforementioned non-competitive or competitive nucleic acid based signal transducer, respectively when operably linked. In most embodiments, the above nucleic acid sequences has one or more reactive groups (as adaptors) linked to suitable locations that can be used to conjugate the recognition elements and sometimes the detectable labels or other components. In some embodiments, the pre-probes may further include at least one adaptors including those having one or more cleavage sites particularly needed for the cleavage probe format. Typically the adaptors have at least one reactive group that can be covalently or non-covalently linked to the reactive groups included in the nucleic acid sequences by using known chemical or biochemical coupling schemes. As discussed, examples of such reactive groups include hydroxyl, carboxyl, amino, sulfflydryl, aldehyde, phosphate, alkyl amino, hydroxy alkyl, alkyl phosphate and the like. In addition and optionally, the pre-probes may further include additional nucleic acid sequences consisting of a series of object or complement sequences having different lengths or different numbers of complementary base pairs. Such additional nucleic acid sequences are especially useful when optimization of the probe operation conditions, particularly the detection temperature, is needed to adjust to the conditions for the interaction of the desired target agent and recognition element. Preferred kits comprising the pre-probes will include means and instructions for converting the pre-probes into desired affinity or reaction probes.

The probe included in the kit may be an immobilized probe tethered to a support according to the invention. In a preferred embodiment, the kit may comprise at least one additional immobilized probe according to the invention having different target agent and immobilized to the same support at a predetermined location. This type of kits having a plurality of immobilized probes for different target agents is very useful for high throughput detection of multiple target agents or high throughput screening of inhibitors or enhancers for multiple target agents. In another preferred embodiment, the kit including an immobilized probe may comprise at least one additional immobilized probe according to the invention having a same target agent and immobilized to the same support at a different predetermined location. This type of kits having a plurality of immobilized probes having a same target agent is very useful for high throughput screening of inhibitors or enhancers for a target agent.

All documents mentioned herein are incorporated by reference.

The present invention is further illustrated by the following Examples. These Examples are provided to aid in the understanding of the invention and are not construed as a limitation thereof.

### EXAMPLE

### Materials

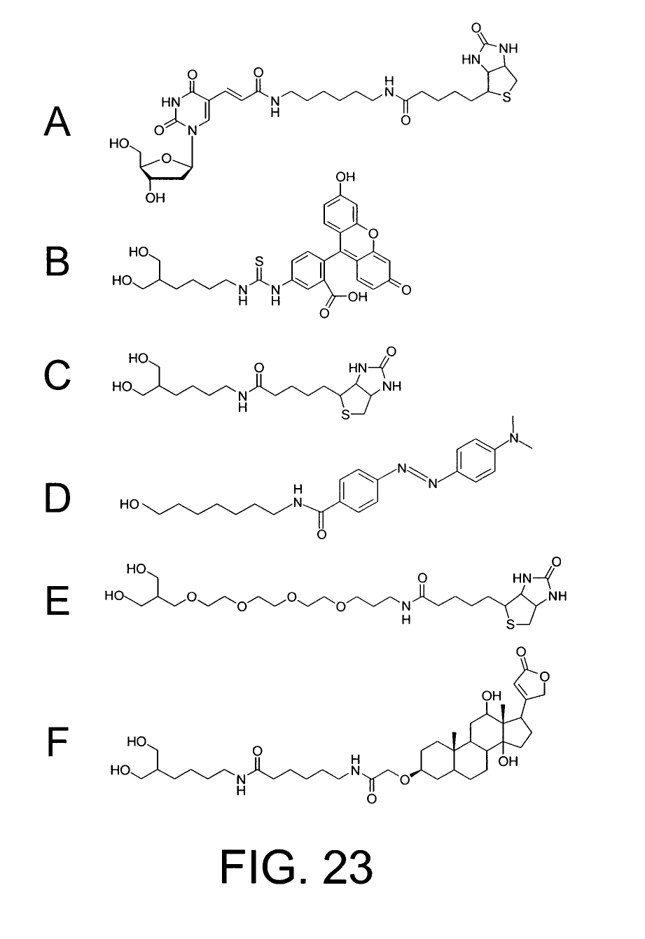

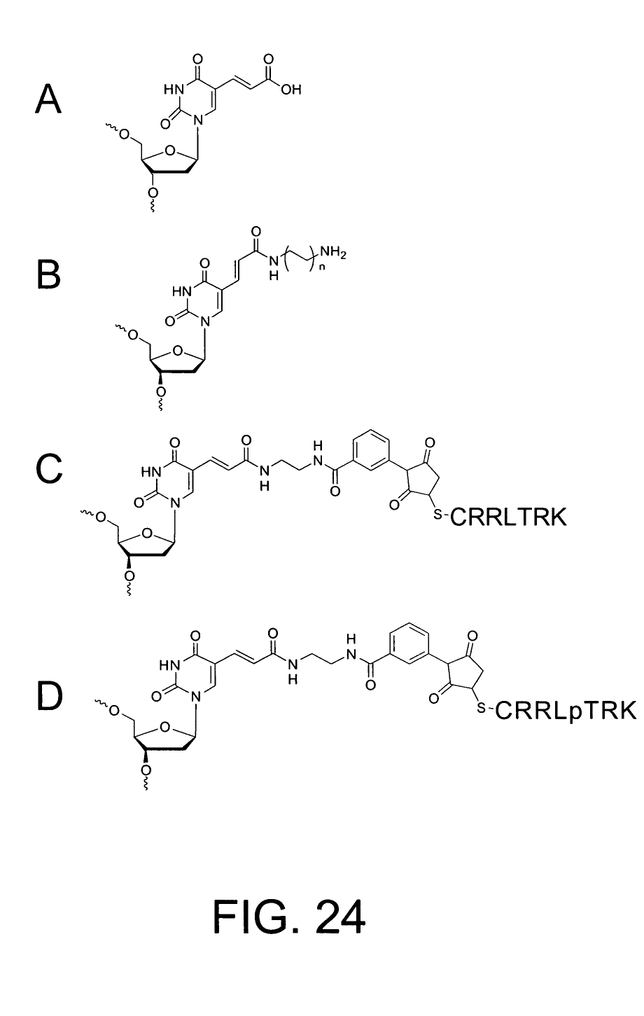

Oligonucleotides used in Example 1-10 were purchased from QIAGEN Operon (CA, USA) and Bioneer (Daejeon, Korea), and those used in Example 11-21 were synthesized using an Expedite 8909 DNA synthesizer (Perceptive Biosystems, MA, USA) . Nucleic acid sequences of the oligonucleotides are provided in the sequence list and the structures of the modified nucleotides contained in the nucleic acid sequences are shown in FIGS. 23 and 24.

In Example 1-9, each oligonucleotide stock solution was prepared at a concentration of 100 μM in TE buffer (10 mM Tris, 1 mM EDTA, pH 8.5), and assays according to the present invention were carried out in STE buffer (100 mM NaCl, 10 mM Tris, 1 mM EDTA, pH 7.5). The results of the assays were confirmed with electrophoresis under non-denaturing conditions using “15% acrylamide TBE Read-gel” from Bio-Rad (CA, USA) in 0.5×TBE buffer (45 mM Tris-borate, 1 mM EDTA). Streptavidin (SA) and goat anti-biotin antibody (AB) were purchased from Pierce (IL, USA), and anti-digoxin antibody (AD) was purchased from Sigma-Aldrich (MO, USA). Streptavidin and antibodies were dissolved in STE buffer or in DI-water at a concentration of 2.5 and 2.0 mg/ml, respectively.

Microwell plates coated with streptavidin used to prepare immobilized probes in Example 9 and 10 were purchased from Pierce. Alkaline phosphatase-linked anti-fluorescein detection kit used in Example 10 was purchased from Amersham Biosciences (NJ, USA).

Phosphoramidite monomers including those with modified nucleotides and CPG supports used to synthesize the oligonucleotides and the probes used in Example 11-21 were purchased from Glen Research (VA, USA). Chemicals and materials used in Example 21 were purchased from the following sources. MBS (m-Maleimidobenzoyl-N-hydroxysuccinimide ester). was purchased from Pierce (IL, USA) and peptides were from Peptron Co. (Daejeon, Korea). Sephadex G-25 was purchased from Amersham Bioscience (Uppsala, Sweden), PKC (Protein Kinase C) from Calbiochem(CA, USA), and anti-phosphothreonine monoclonal antibody and staurosporin from Sigma-Aldrich (MO, USA).

### Example 1

### Assay for Detecting Streptavidin Using a Biotin-Coupled Bimolecular Probe

A bimolecular affinity probe having a biotin as a probe ligand was used to detect a receptor agent, streptavidin (SA). The bimolecular probe used in this example consists of an object oligonucleotide OB1-39B (SEQ ID NO: 123) comprising a first object sequence and a probe ligand, biotin, and a first complement oligonucleotide CM1* (SEQ ID NO: 124) comprising a first complement sequence and a fluorescent label, fluorescein. The probe ligand is covalently linked to the 39th nucleotide (thymidine) of the object oligonucleotide (see FIG. 23A for the structure of the biotin-coupled thymidine). Fluorescein is covalently linked to the first nucleotide (guanosine) of the first complement oligonucleotide by a linker (see FIG. 23B for the structure of the fluorescein with the linker).

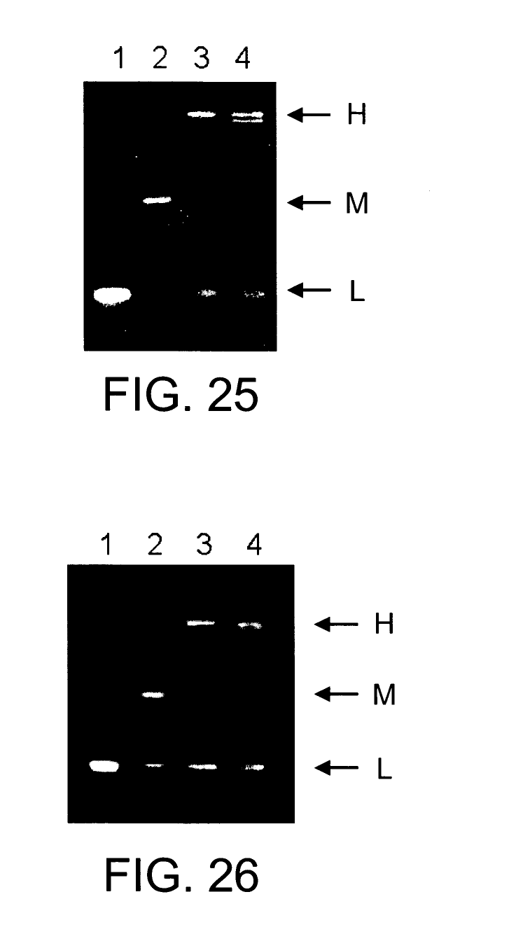

Four assay solutions were prepared according to the compositions summarized in Table 4, and incubated at room temperature for an hour to permit binding of streptavidin to the probe ligand. The results were analyzed by electrophoresis under non-denaturing conditions. As shown in FIG. 25, in the absence of streptavidin, the first object and first complement oligonucleotides hybridized to form a first hybridized duplex (the band M in lane 2). A very weak band was observed for the unhybridized first complement oligonucleotide (the band L in lane 2), indicating that the first hybridized duplex was thermodynamically stable under the assay conditions including the detection temperature in the absence of streptavidin. In the presence of streptavidin, binding of streptavidin to the probe ligand caused destabilization of the first hybridized duplex, resulting in dissociation of the first hybridized duplex. This is evidenced by the increased intensity of the low mass band (the band L in lane 3 and 4) corresponding to the dissociated first complement oligonucleotide CM1*, and also by the occurrence of the high mass band (the band H in lane 3 and 4) corresponding to the first hybridized duplex complexed with streptavidin. The multiple bands observed in the high mass region of lane 3 and 4 are due to the fact that streptavidin has four binding sites for binding of biotin.

In this type of bimolecular probes, the presence of the target agent can be detected by monitoring the level of the characteristic signal from the label associated with the dissociated first complement sequence or the first hybridized duplex. Detecting the signal from the first hybridized duplex is especially useful if the object oligonucleotide is immobilized to a support as in Example 9.

TABLE 4
Compositions of the assay solutions used in Example 1.

(unit: μl)

| Assay No. | 1 | 2 | 3 | 4 |
|---|---|---|---|---|
| OB1-39B | — | 2.0 | 2.0 | 2.0 |
| CM1* | 2.0 | 2.0 | 2.0 | 2.0 |
| SA | — | — | 2.8 | 5.6 |
| STE Buffer | 18.0 | 16.0 | 13.2 | 10.4 |

### Example 2

### Assay for Detecting Streptavidin Using a Biotin-Coupled Bimolecular Probe Having an Abasic Nucleotide

A bimolecular affinity probe similar to the probe used in Example 1 was used to detect a receptor agent, streptavidin (SA). The bimolecular probe used in this example consists of an object oligonucleotide OB2-39B (SEQ ID NO: 125) comprising a first object sequence and a probe ligand, biotin, and a first complement oligonucleotide CM2* (SEQ ID NO: 126) comprising a first complement sequence and a fluorescent label, fluorescein. The probe ligand is covalently linked to the 39th abasic nucleotide of the object oligonucleotide (see FIG. 23C for the structure of the biotin-coupled abasic nucleotide). Due to this abasic nucleotide, there exists a mismatch (or un-match) in the hybridization of the first object sequence and the first complement sequence, different from the probe used in Example 1. Fluorescein is covalently linked to the first nucleotide (guannosine) of the first complement oligonucleotide (see FIG. 23B for the structure of the fluorescein with the linker).

As in Example 1, four assay solutions were prepared according to the compositions summarized in Table 5, and incubated at room temperature for an hour to permit binding of streptavidin to the probe ligand. The results were analyzed by electrophoresis. As shown in FIG. 26, in the absence of streptavidin, the first object and first complement oligonucleotides hybridized to form a first hybridized duplex (the band M in lane 2). The unhybridized first complement oligonucleotide is observed with a higher intensity (the band L in lane 2) compared to Example 1. This is due to the mismatch in the hybridization of the first object and first complement sequences. In the presence of streptavidin, binding of streptavidin to the probe ligand caused destabilization of the first hybridized duplex, resulting in dissociation of the first hybridized duplex. This is evidenced by the increased intensity of the low mass band (the band L in lane 3 and 4) corresponding to the dissociated first complement oligonucleotide CM2*, and also by the occurrence of the high mass band (the band H in lane 3 and 4) corresponding to the first hybridized duplex complexed with streptavidin. The observed change in the amount of the dissociated first complement oligonucleotide upon binding of the receptor agent was smaller than that observed with the probe used in Example 1. This suggests that the destabilization effect may be relatively weaker when the probe ligand is coupled to an abasic nucleotide as in this example.

TABLE 5
Compositions of the assay solutions used in Example 2.

(unit: μl)

| Assay No. | 1 | 2 | 3 | 4 |
|---|---|---|---|---|
| OB2-39B | — | 2.0 | 2.0 | 2.0 |
| CM2* | 2.0 | 2.0 | 2.0 | 2.0 |
| SA | — | — | 2.8 | 5.6 |
| STE Buffer | 18.0 | 16.0 | 13.2 | 10.4 |

### Example 3

### Assay for Detecting Streptavidin Using Unimolecular Probes with an Interactive Label Pair Consisting of a Fluorescer and a Quencher

Two unimolecular affinity probes, UP1-34B (SEQ ID NO: 127) and UP2-6B/36B (SEQ ID NO: 128), having biotin as a probe ligand were used to detect a receptor agent, streptavidin (SA). The unimolecular affinity probe UP1 is depicted schematically in FIG. 18. UP1 has a biotin-coupled thymidine at the 34th position, and UP2 has two biotin-coupled thyrnidines at the 6th and 36th positions (see FIG. 23A for the structure of the biotin-coupled thymidine). Each unimolecular probe has an interactive label pair consisting of a fluorescer, fluorescein, and a quencher, DABCYL (4-(4′-dimethylaminophenylazo)benzoic acid), which are covalently linked to the 5′ and the 3′ terminus by a linker, respectively. The structures of the fluorescein and DABCYL with the linker are shown in FIGS. 23B and 23D, respectively. UP1 and UP2 are designed to form hybridized conformations having stem-loop structures in the absence of the receptor agent, by forming stem duplexes having 6 and 7 hybridized base pairs, respectively, between the 5′ and 3′ terminal regions. The 5′ and 3′ terminal regions act as the first object and first complement sequences. When the stem-loop structure is formed, the fluorescer moiety becomes located in close proximity to the quencher moiety, leading to efficient interaction between the two moieties. This causes decrease of the fluorescence intensity from the probe compared to the dissociated conformation in which the stem duplex is dissociated and thus the fluorescer moiety is located distal from the quencher moiety. A loop binding oligonucleotide, LB1 (SEQ ID NO 129), that is complementary to the loop region and causes dissociation of the stem duplex upon hybridization to the loop region, was used as a control.

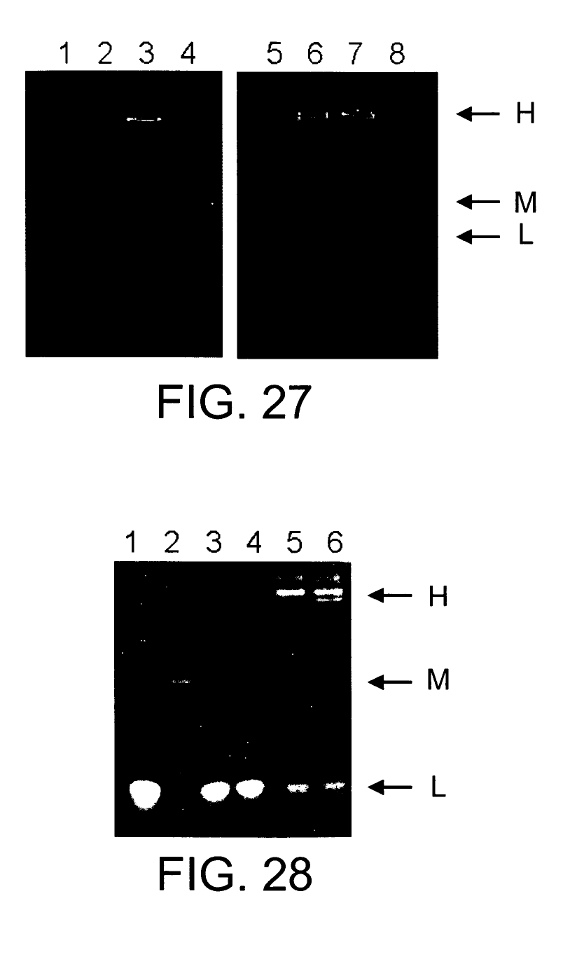

Assay solutions were prepared according to the compositions summarized in Table 6, incubated in ice for an hour, and analyzed by electrophoresis. As shown in FIG. 27, in the absence of streptavidin, weak fluorescence was observed at the position corresponding to the size of the unimolecular probes (the band L in lane 1 for UP1 and lane 5 for UP2). This indicates that both probes formed the stem-loop structures, causing efficient quenching of the fluorescer by the quencher located in close proximity. In the presence of streptavidin, strongly enhanced fluorescence was observed in the high mass region (the band H in lane 2 and 3 for UP1 and lane 6 and 7 for UP2), indicating binding of streptavidin to the probe ligand which caused dissociation of the stem duplexes. As in previous examples, the multiple bands observed in the high mass region are due to the presence of the multiple binding sites in streptavidin. Dissociation of the stem duplexes due to the destabilization effect caused by the binding of the receptor agent is manifested by the increased fluorescence. The stem duplexes dissociate upon binding of the receptor agent and the unimolecular probes possess the dissociated conformations in which the fluorescer becomes located distant from the quencher, thereby emitting increased fluorescence. Lane 4 and 8 show the results obtained when the loop binding oligonucleotide LB1 was mixed with the unimolecular probes. LB1 binds to the loop regions of the unimolecular probes and causes the unimolecular probes to possess the dissociated conformations. The fact that the increase of the fluorescence intensity observed in the presence of streptavidin is similar to that observed in the presence of the loop binding oligonucleotide indicates that binding of streptavidin strongly destabilized the stem duplexes of the unimolecular probes.

The results observed in this example suggests that presence of the receptor agent can be detected by simply monitoring the fluorescence intensity without the electrophoretic separation process, when the interactive label pair consisting of a fluorescer and a quencher is used. Therefore, this example demonstrates that the probes having at least one interactive label pair according to the present invention can be used in homogeneous assays as well as heterogeneous assays for detecting target agents.

TABLE 6
Compositions of the assay solutions used in Example 3.

(unit: μl)

| Assay No. | 1 | 2 | 3 | 4 | 5 | 6 | 7 | 8 |
|---|---|---|---|---|---|---|---|---|
| UP1-34B | 5.0 | 5.0 | 5.0 | 5.0 | | | | |
| UP2-6B/36B | | | | | 5.0 | 5.0 | 5.0 | 5.0 |
| SA | — | 0.1 | 0.5 | — | — | 0.1 | 0.5 | — |
| LB1 | — | — | — | 2.0 | — | — | — | 2.0 |
| STE Buffer | 15.0 | 14.9 | 14.5 | 13.0 | 15.0 | 14.9 | 14.5 | 13.0 |

### Example 4

### Assay for Detecting Streptavidin Using a Trimolecular Probe Having a Probe Ligand Conjugated to the First Object Sequence

A trimolecular affinity probe having a probe ligand (biotin) conjugated to the first object sequence was used to detect a receptor agent, streptavidin. The trimolecular probe used in this example is depicted schematically in FIG. 13. The trimolecular probe consists of an object oligonucleotide OB1-39B (SEQ ID NO: 123) comprising first and second object sequences and a probe ligand conjugated to the first object sequence, a first complement oligonucleotide CM1 (SEQ ID NO: 130) that is complementary to the first object sequence, and a second complement oligonucleotide CM3* (SEQ ID NO: 131) that is complementary to the second object sequence. The probe ligand is covalently linked to the 39th nucleotide, thymidine of the object oligonucleotide (see FIG. 23A for the structure of the biotin-coupled thymidine). The second complement oligonucleotide CM3* has a fluorescent label, fluorescein, covalently linked to the first nucleotide, cytidine by a linker (see FIG. 23B for the structure of the fluorescein with the linker).

In this probe, the two complement oligonucleotides CM1 and CM3* competitively hybridize with the object oligonucleotide because the first and second object sequences have an overlapping region in common. Since the first hybridized duplex between the first object sequence and the first complement sequence has two more complementary base pairs than the second hybridized duplex between the second object sequence and the second complement sequence, the first hybridized duplex is preferentially formed in the absence of the receptor agent. Note that the probe ligand is conjugated to the first object sequence but not to the second object sequence according to the preferred embodiment of the present invention. This leads to the differential destabilization, i.e., binding of the receptor agent to the probe ligand causes a stronger destabilization of the first hybridized duplex and a weaker or no destabilization of the second hybridized duplex.

Assay solutions were prepared according to the compositions in Table 7, incubated at room temperature for an hour, and analyzed by electrophoresis. As shown in FIG. 28, in the absence of streptavidin, the first complement oligonucleotide CM1 hybridized preferentially to the object oligonucleotide, causing most of the second complement oligonucleotide CM3* remaining unhybridized (e.g., compare the band L in lane 3 and 4 with that in lane 1). The control experiment shown in lane 2 indicates that the second complement oligonucleotide can hybridize efficiently to the object oligonucleotide in the absence of the first complement oligonucleotide, as evidenced by the occurrence of the band M and the decrease of the intensity of the band L in lane 2 as compared to lane 3 and 4. As shown in lane 5 and 6 of FIG. 28, in the presence of streptavidin, the high mass band corresponding to the second hybridized duplex complexed with streptavidin (the band H) appeared and the intensity of the unhybridized second complement oligonucleotide (the band L) decreases dramatically compared to that in the absence of streptavidin. This observation indicates that binding of streptavidin destabilized the first hybridized duplex more strongly than the second hybridized duplex. Therefore, in the presence of the receptor agent, the second complement oligonucleotide hybridized preferentially to the object oligonucleotide compared to the first complement oligonucleotide, resulting in formation of a large amount of the second hybridized duplex complexed with streptavidin (the band H in lane 5 and 6). The large decrease observed in the amount of the unhybridized second complement oligonucleotide (the band L in lane 5 and 6) is also consistent with this explanation. The fact that the intensity of the band L in lane 5 and 6 are relatively higher than that in lane 2 suggests that binding of streptavidin to the probe ligand likely caused destabilization of the second hybridized duplex, but relatively weakly compared to the first hybridized duplex.

Using this type of trimolecular probes having at least one non-interactive label conjugated to at least one of the first and second complement sequences, the presence of the target agent can be detected by monitoring the characteristic signal from the label associated with the hybridized duplexes or the unhybridized sequences. Detecting the signal from the hybridized duplexes is especially useful if the object oligonucleotide is immobilized to a support as in Example 9.

This type of trimolecular probes can be advantageous compared to the bimolecular probes used in Example 1 and 2. Because of the differential destabilization effect embedded in this type of the trimolecular probes, the first and second complement sequences compete differentially in the hybridization process depending on the presence or absence of the receptor agent. For instance, in the case of the probe used in this example, the first hybridized duplex is preferentially formed in the absence of the receptor agent, but the second hybridized duplex is preferentially formed in the presence of the receptor agent. This differential competition induced by the differential destabilization can lead to an enhanced signal compared to the bimolecular probes used in Example 1 and 2, because dissociation of the first hybridized duplex due to the receptor binding can be aided by the preferential hybridization of the second complement sequence in the presence of the receptor agent.

TABLE 7
Compositions of the assay solutions used in Example 4.

(unit: μl)

| Assay No. | 1 | 2 | 3 | 4 | 5 | 6 |
|---|---|---|---|---|---|---|
| OB1-39B | — | 2.0 | 2.0 | 2.0 | 2.0 | 2.0 |
| CM1 | — | — | 2.0 | 4.0 | 2.0 | 2.0 |
| CM3* | 2.0 | 2.0 | 2.0 | 2.0 | 2.0 | 2.0 |
| SA | — | — | — | — | 2.8 | 5.6 |
| STE Buffer | 18.0 | 16.0 | 14.0 | 12.0 | 11.2 | 8.4 |

### Example 5

### Assay for Detecting Streptavidin Using a Trimolecular Probe Having a Probe Ligand Conjugated to the First Complement Sequence

A trimolecular affinity probe having a probe ligand (biotin) conjugated to the first complement sequence was used to detect a receptor agent, streptavidin. The trimolecular probe consists of an object oligonucleotide OB1 (SEQ ID NO: 132) comprising first and second object sequences, a first complement oligonucleotide CM1-4B (SEQ ID NO: 133) that is complementary to the first object sequence and has a probe ligand, and a second complement oligonucleotide CM3* (SEQ ID NO: 131) that is complementary to the second object sequence. The probe ligand is covalently linked to the 4th nucleotide, thymidine of the first complement oligonucleotide (see FIG. 23A for the structure of the biotin-coupled thymidine). The second complement oligonucleotide has a fluorescent label, fluorescein, covalently linked to the first nucleotide, cytidine by a linker (see FIG. 23B for the structure of the fluorescein with the linker).

As in the case of the probe used in Example 4, the two complement oligonucleotides CM1-4B and CM3* compete to hybridize with the object oligonucleotide. However, only the first hybridized duplex is under the influence of the destabilization effect because the probe ligand is conjugated to the first complement sequence. Destabilization of the first hybridized duplex by the receptor binding will result in preferential formation of the second hybridized duplex.

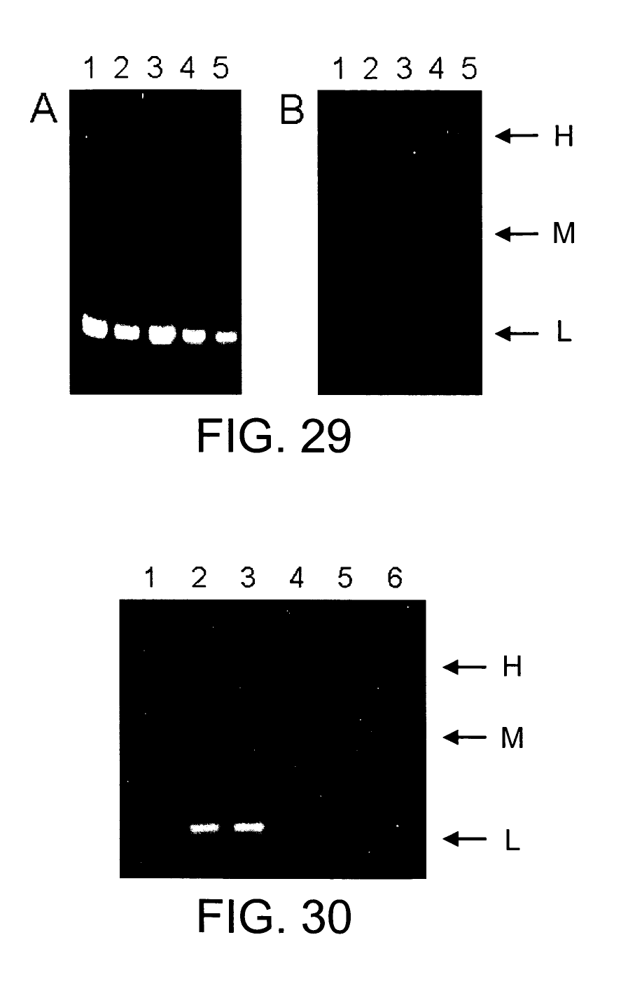

As in the previous examples, the assay solutions were prepared according to the compositions in Table 8, and analyzed by electrophoresis after incubating at room temperature for an hour. As shown in FIG. 29A, in the absence of streptavidin, the first hybridized duplex was preferentially formed, and thus most of the second complement oligonucleotide remained unhybridized (see the band L in lane 3). As shown in lane 2, in the absence of the first complement oligonucleotide, the second complement sequence can hybridize efficiently with the object sequence (see the band M in lane 2). In the presence of streptavidin, the second hybridized duplex was preferentially formed (the band M in lane 4 and 5) and the intensity of the unhybridized second complement sequence decreased. This is due to the destabilization of the first hybridized duplex by the receptor binding.

FIG. 29B shows the results obtained with ethidium bromide staining. In the absence of streptavidin, preferential formation of the first hybridized duplex, which was not visible in FIG. 29A because no detectable label was associated, was observed (the band M in lane 3). The band H observed in lane 4 and 5 corresponds to the first hybridized duplex complexed with streptavidin, which was not visible in FIG. 29A. The band M in lane 4 and 5 corresponds to the second hybridized duplex that was also visible in FIG. 29A.

TABLE 8
Compositions of the assay solutions used in Example 5.

(unit: μl)

| Assay No. | 1 | 2 | 3 | 4 | 5 |
|---|---|---|---|---|---|
| OB1 | — | 2.0 | 2.0 | 2.0 | 2.0 |
| CM1-4B | — | — | 2.0 | 2.0 | 2.0 |
| CM3* | 2.0 | 2.0 | 2.0 | 2.0 | 2.0 |
| SA | — | — | — | 2.8 | 5.6 |
| STE Buffer | 18.0 | 16.0 | 14.0 | 11.2 | 8.4 |

### Example 6

### Assay for Detecting Streptavidin Using a Trimolecular Probe Having Probe Ligands Conjugated to Both the First Object and First Complement Sequences

A trimolecular affinity probe having two probe ligands (biotin) conjugated to the first object and first complement sequences, were used to detect a receptor agent, streptavidin. The trimolecular probe consists of an object oligonucleotide OB1-39B (SEQ ID NO: 123) comprising first and second object sequences and a probe ligand, a first complement oligonucleotide CM1-4B (SEQ ID NO: 133) that is complementary to the first object sequence and has a probe ligand, and a second complement oligonucleotide CM3* (SEQ ID NO: 131) that is complementary to the second object sequence. One of the probe ligands is covalently linked to the 39th nucleotide, thymidine of the object oligonucleotide, and the other to the 4th nucleotide, thymidine of the first complement oligonucleotide (see FIG. 23A for the structure of the biotin-coupled thymidine). The second complement oligonucleotide has a fluorescent label, fluorescein, covalently linked to the first nucleotide, cytidine by a linker (see FIG. 23B for the structure of the fluorescein with the linker).

As in the previous examples, the assay solutions were prepared according to the compositions in Table 9, and analyzed by electrophoresis after incubating at room temperature for an hour. As shown in FIG. 30, in the absence of streptavidin, the first hybridized duplex was preferentially formed, and thus most of the second complement oligonucleotide remained unhybridized (see the band L in lane 2 and 3). When the first complement oligonucleotide was not present, the second complement oligonucleotide hybridized efficiently as observed in the band M in lane 1. In the presence of streptavidin, the second hybridized duplex was preferentially formed (the band H in lane 4-6) and the intensity of the unhybridized second complement oligonucleotide decreased (the band L in lane 4-6). This is due to the destabilization of the first hybridized duplex by the receptor binding. Note that different from Example 5, the second hybridized duplex was observed in the high mass region in the presence of streptavidin, because streptavidin was bound to the object sequence.

TABLE 9
Compositions of the assay solutions used in Example 6.

(unit: μl)

| Assay No. | 1 | 2 | 3 | 4 | 5 | 6 |
|---|---|---|---|---|---|---|
| OB1-39B | 2.0 | 2.0 | 2.0 | 2.0 | 2.0 | 2.0 |
| CM1-4B | — | 2.0 | 4.0 | 2.0 | 2.0 | 4.0 |
| CM3* | 2.0 | 2.0 | 2.0 | 2.0 | 2.0 | 2.0 |
| SA | — | — | — | 2.8 | 5.6 | 5.6 |
| STE Buffer | 16.0 | 14.0 | 12.0 | 11.2 | 8.4 | 6.4 |

### Example 7

### Assay for Detecting Streptavidin Using a Trimolecular Probe Having a Probe Ligand Conjugated to an Abasic Nucleotide in the First Object Sequence

A trimolecular probe used in this example is similar to the probe used in Example 4, except that the probe ligand is covalently linked to an abasic nucleotide. The probe used in this example consists of an object oligonucleotide OB2-39B (SEQ ID NO: 125) comprising first and second object sequences and a probe ligand conjugated to the first object sequence, a first complement oligonucleotide CM1 (SEQ ID NO: 130) that is complementary to the first object sequence, and a second complement oligonucleotide CM3* (SEQ ED NO: 131) that is complementary to the second object sequence. The probe ligand is covalently linked to the 39th abasic nucleotide of the object oligonucleotide (see FIG. 23C for the structure of the biotin-coupled abasic nucleotide). The second complement oligonucleotide has a fluorescent label, fluorescein, covalently linked to the first nucleotide, cytidine by a linker (see FIG. 23B for the structure of the fluorescein with the linker).

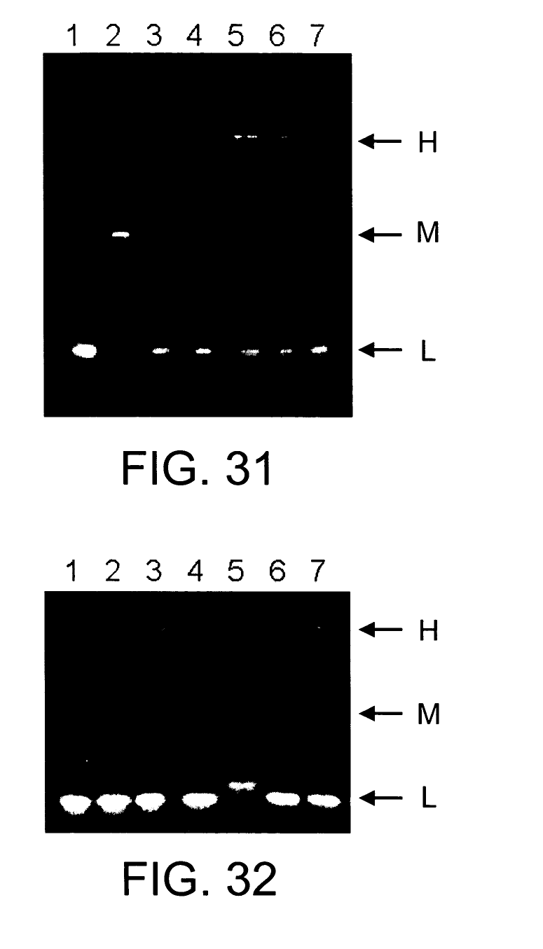

Assay solutions were prepared according to the compositions in Table 10, analyzed by electrophoresis after incubating at room temperature for an hour. As shown in FIG. 31, the results are nearly identical to those of Example 4. In the absence of streptavidin, the first complement oligonucleotide CM2 hybridized preferentially and most of the second complement oligonucleotide CM3* remained unhybridized as shown in lane 3 and 4. When the first complement oligonucleotide was not present, the second complement oligonucleotide hybridized efficiently as observed in the band M in lane 2. In the presence of streptavidin, the high mass band (the band H) corresponding to the second hybridized duplex complexed with streptavidin appeared and the intensity of the unhybridized second complement oligonucleotide (the band L) decreased. Therefore, the presence of the receptor agent can be detected by monitoring the characteristic signal from the label associated with the second hybridized duplex or the unhybridized second complement oligonucleotide.

TABLE 10
Compositions of the assay solutions used in Example 7.

(unit: μl)

| Assay No. | 1 | 2 | 3 | 4 | 5 | 6 | 7 |
|---|---|---|---|---|---|---|---|
| OB2-39B | — | 2.0 | 2.0 | 2.0 | 2.0 | 2.0 | 2.0 |
| CM2 | — | — | 2.0 | 4.0 | 2.0 | 2.0 | 4.0 |
| CM3* | 2.0 | 2.0 | 2.0 | 2.0 | 2.0 | 2.0 | 2.0 |
| SA | — | — | — | — | 2.8 | 5.6 | 5.6 |
| STE Buffer | 18.0 | 16.0 | 14.0 | 12.0 | 11.2 | 8.4 | 6.4 |

### Example 8

### Assay for Detecting an Antibody Using Trimolecular Probes

Two trimolecular affinity probes used in this example are the probes used in Example 4 and 7, but the receptor agent is selected to be goat anti-biotin antibody (AB). The first probe that was used in Example 4 consists of an object oligonucleotide OB1-39B (SEQ ID NO: 123), a first complement sequence CM1 (SEQ ID NO: 130), and a second complement sequence CM3* (SEQ ID NO: 131). The second probe that was used in Example 7 consists of an object oligonucleotide OB2-39B (SEQ ID NO: 125), a first complement sequence CM2 (SEQ ID NO: 134), and a second complement sequence CM3* (SEQ ID NO: 131).

As in previous examples, the assay solutions were prepared according to the compositions in Table 11, and analyzed by electrophoresis after incubating at room temperature for an hour. As shown in FIG. 32, in the absence of the receptor agent, the first complement oligonucleotide hybridized preferentially to the object oligonucleotide in both probes. Therefore, as shown in lane 1 and 4, most of the second complement oligonucleotide remained unhybridized (the band L) and very small amount of the second hybridized duplex (the band M) was formed. In the presence of the receptor agent, binding of the receptor agent destabilized the first hybridized duplex, and thus the second hybridized duplex was formed preferentially. This is observed in lane 2 and 3 for the first probe and in lane 6 and 7 for the second probe. The high mass band H corresponds to the second hybridized duplex complexed with anti-biotin antibody. The decrease in the amount of the unhybridized second complement oligonucleotide (the band L) is less evident compared to the previous examples, suggesting that the destabilization effect of anti-biotin antibody is relatively weaker than that of streptavidin. Lane 5 shows a control assay in which a bimolecular probe with a first complement oligonucleotide CM2* (SEQ ID NO: 126) having a fluorescent label is used. In this control assay using the bimolecular probe, the high mass band (the band H) corresponds to the first hybridized duplex complexed with anti-biotin antibody. The band M and L are the first hybridized duplex and the unhybridized first complement oligonucleotide, respectively.

TABLE 11
Compositions of the assay solutions used in Example 8.

(unit: μl)

| Assay No. | 1 | 2 | 3 | 4 | 5 | 6 | 7 |
|---|---|---|---|---|---|---|---|
| OB1-39B | 2.0 | 2.0 | 2.0 | — | — | — | — |
| OB2-39B | — | — | — | 2.0 | 2.0 | 2.0 | 2.0 |
| CM1 | 4.0 | 2.0 | 2.0 | — | — | — | — |
| CM2 | — | — | — | 4.0 | 2.0⁽¹⁾ | 2.0 | 2.0 |
| CM3* | 2.0 | 2.0 | 2.0 | 2.0 | | 2.0 | 2.0 |
| AB | — | 5.0 | 10.0 | — | 5.0 | 5.0 | 10.0 |
| STE Buffer | 12.0 | 9.0 | 4.0 | 12.0 | 9.0 | 9.0 | 4.0 |

⁽¹⁾CM2* (SEQ ID NO: 126) was used instead of CM2 (SEQ ID NO: 134) to visualize the first hybridized duplex.

### Example 9

### Assay for Detecting Streptavidin Using Immobilized Trimolecular Probes Having a Probe Ligand Conjugated to the First Complement Sequence

Two immobilized trimolecular affinity probes having a probe ligand (biotin) conjugated to the first complement sequence were used to detect streptavidin. One of the trimolecular probes used in this example is depicted schematically in FIG. 15. The first probe consists of an object oligonucleotide OB1-1B (SEQ ID NO: 135) comprising first and second object sequences, a first complement sequence CM2-4B (SEQ ID NO: 136) having a probe ligand, and a second complement sequence CM3* (SEQ ID NO: 131). The second probe consists of an object oligonucleotide OB1-1B (SEQ ID NO: 135), a first complement sequence CM1-4B (SEQ ID NO: 133) having a probe ligand, and a second complement sequence CM3* (SEQ ID NO: 131). The object oligonucleotide OB1-1B has a biotin covalently linked to the first nucleotide, adenosine in the 5′ end that is used to immobilize the object sequence (see FIG. 23E for the structure of the biotin-coupled abasic nucleotide). The second complement oligonucleotide CM3* has fluorescein as a fluorescent label at the 5′ terminal.

The object oligonucleotide having a biotin residue in the 5′ terminal was immobilized to microwell plate wells coated with streptavidin. For immobilization, 90 μl of STE buffer and 10 μl of the object oligonucleotide solution (1 μM) were loaded to each well, and washed twice with 150 μi of STE buffer after shaking for 30 min at room temperature. Other reagents were then added to each well according to the compositions described in Table 12. After 30 min incubation at room temperature, each well was washed twice with 150 μl of STE buffer to remove unbound species, and 100 μl of 0.2 N NaOH solution was added to each well to release the hybridized second complement oligonucleotide CM3* to the solution phase. Fluorescence from the second complement oligonucleotide CM3* from each well was then measured at 535 nm using a microwell plate reader (GENios, TECAN, USA).

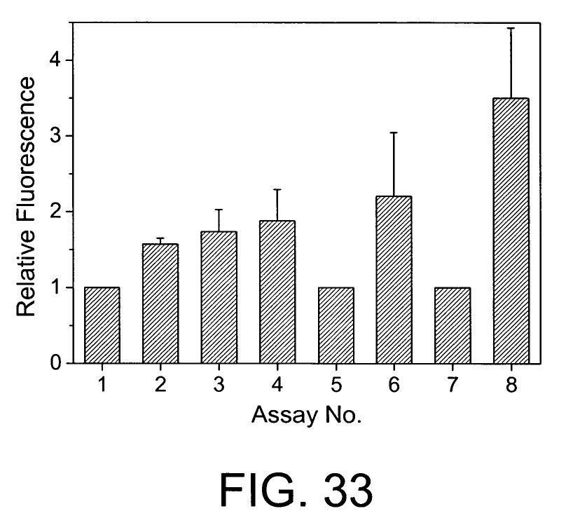

The results are presented in FIG. 33. The fluorescence intensities shown in FIG. 33 are normalized with respect to the intensity observed in the absence of the receptor agent for each probe. As shown in FIG. 33, addition of streptavidin leads to increase of the fluorescence intensity. For example, in the case of the first probe (assay No. 1-4), the fluorescence intensity increased by 1.5-1.9 fold depending on the amount of streptavidin added. In the case of the second probe (assay No. 5-8), the fluorescence intensity increased by a factor of 2-3.5 upon addition of streptavidin. The observed increase of the fluorescence indicates that binding of the receptor agent to the probe ligand destabilized the first hybridized duplex, resulting in preferential hybridization of the second complement oligonucleotide, which has the fluorescent label, with the immobilized object oligonucleotide.

TABLE 12
Compositions of the assay solutions used in Example 9.

(unit: μl)

| Assay No. | 1 | 2 | 3 | 4 | 5 | 6 | 7 | 8 |
|---|---|---|---|---|---|---|---|---|
| OB1-1B | 10 | 10 | 10 | 10 | 10 | 10 | 10 | 10 |
| CM2-4B | 10 | 10 | 10 | 10 | — | — | — | — |
| CM1-4B | — | — | — | — | 10 | 10 | 20 | 20 |
| CM3* | 10 | 10 | 10 | 10 | 10 | 10 | 10 | 10 |
| SA | — | 2.5 | 10 | 40 | — | 10 | — | 10 |
| STE Buffer | 80 | 77.5 | 70 | 40 | 80 | 70 | 70 | 60 |

### Example 10

### Assay for Detecting an Antibody Using an Immobilized Trimolecular Probe Having a Label for ELISA

An immobilized trimolecular affinity probe having a digoxigenin as a probe ligand was used to detect anti-digoxin antibody (AD). The probe consists of an object oligonucleotide OB1-1B/39D (SEQ ID NO: 137) comprising first and second object sequences and a probe ligand, digoxigenin, a first complement sequence CM2 (SEQ ID NO: 134), and a second complement sequence CM3* (SEQ ID NO: 131). The object oligonucleotide OB1-1B/39D has a digoxigenin covalently linked to the 39th abasic nucleotide (see FIG. 23F for the structure of the digoxigenin-coupled abasic nucleotide) and a biotin covalently linked to the 5′ terminal. The second complement oligonucleotide CM3* has fluorescein as a fluorescent label at the 5′ terminal.

The object oligonucleotide was immobilized to microwell plate wells coated with streptavidin using the biotin residue in the 5′ terminal. The immobilization procedure was the same as Example 9. Other reagents were then added to each well according to the compositions described in Table 13. After incubating for an hour at room temperature, each well was washed twice with 150 μl of STE buffer to remove unbound species, and ELISA assay was performed to measure the amount of the second complement oligonucleotide CM3* hybridized to the immobilized object oligonucleotide. Alkaline phosphatase-linked anti-fluorescein detection kit from Amersham Biosciences was used to detect fluorescein conjugated to the second complement oligonucleotide.

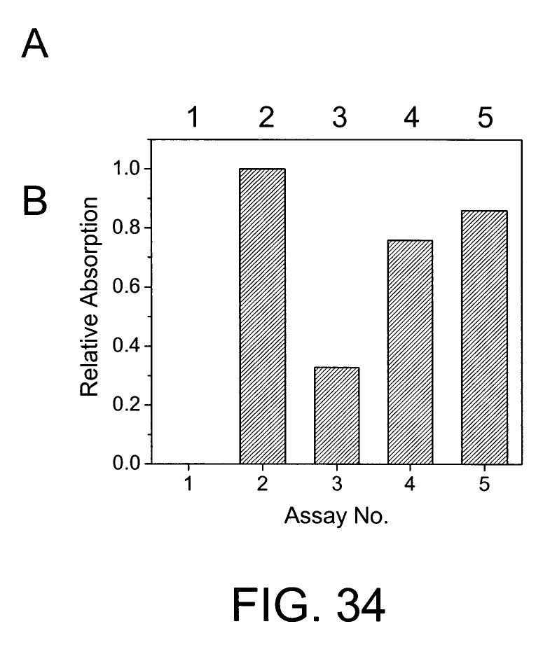

FIG. 34A shows a photograph of a microwell plate taken after the ELISA amplification. Appearance of the yellow color on the edges of the microwells indicates the presence of fluorescein bound to the immobilized object oligonucleotide. Intensity of the yellow color was calculated for each well from the photograph image, and the results are presented in FIG. 34B. When no object oligonucleotide was immobilized (assay No.1), the ELISA signal stayed at zero level. When only the labeled second complement oligonucleotide was added to the immobilized object oligonucleotide (assay No. 2), the ELISA signal increased dramatically, showing the maximum possible signal. Assay No.3 shows that the ELISA signal observed from the trimolecular probe in the absence of the receptor agent (digoxigenin) is about 30% relative to that observed in assay No.2. This observation indicates that the first complement oligonucleotide hybridized preferentially in the absence of the receptor agent, resulting in a low level of hybridization of the second complement oligonucleotide having the detectable label. Upon addition of the receptor agent, the ELISA signal increased by a factor of more than 2 as shown in assay No. 4 and 5, indicating that binding of the receptor agent to the probe ligand destabilized the first hybridized duplex compared to the second hybridized duplex.

TABLE 13
Compositions of the assay solutions used in Example 10.

(unit: μl)

| Assay No. | 1 | 2 | 3 | 4 | 5 |
|---|---|---|---|---|---|
| OB1-1B/39D | — | 1 | 1 | 1 | 1 |
| CM2 | — | — | 1 | 1 | 1 |
| CM3* | 1 | 1 | 1 | 1 | 1 |
| AD | — | — | — | 5 | 10 |
| STE Buffer | 149 | 148 | 147 | 142 | 137 |

### Example 11

### Loop Length Dependence of the Fluorescence Characteristics of the Non-Competitive Unimolecular Probe having an Interactive Label Pair Consisting of a Fluorescer and a Quencher

Six non-competitive unimolecular affinity probes having structures similar to the one depicted in FIG. 17 were synthesized and used to examine loop length dependence of the fluorescence characteristics of the non-competitive unimolecular probe. The six unimolecular probes, UP3-12B (SEQ ID NO: 138), UP4-14B (SEQ ID NO: 139), UP5-16B (SEQ ID NO: 140), UP6-23B (SEQ ID NO: 141), UP7-33B (SEQ ID NO: 142), and UP8-43B (SEQ ID NO: 143), have different number of nucleotides in the loop region, i.e., 4, 6, 8, 15, 25, and 35, respectively, but have the same 5 bp stem duplex sequence (i.e., the sequence being 5′-fluorescein-GCAGG-loop-CCTGC-DABCYL-3′). All of these unimolecular probes have a biotin as a probe ligand (to detect a target receptor agent, streptavidin) linked to the same position within the 3′ terminal stem region, i.e., to the third nucleotide, thymidine from the 3′ terminus (see FIG. 23A for the structure of the biotin-coupled thymidine). Each unimolecular probe has an interactive label pair consisting of a fluorescer, fluorescein, and a quencher, DABCYL (4-(4′-dimethylaminophenylazo)benzoic acid), which are covalently linked to the 5′ and the 3′ terminus by a linker, respectively. The structures of the fluorescein and DABCYL with the linker are shown in FIGS. 23B and 23D, respectively.

All of the unimolecular probes described above are designed to form hybridized conformations having stem-loop structures in the absence of the receptor agent (at the temperature region below the melting temperature of the stem duplex), by forming a stem duplex having 5 hybridized base pairs between the 5′ and 3′ terminal regions. According to the invention, the 5′ and 3′ terminal regions act as the first object and first complement sequences. When the stem-loop structure is formed, the fluorescer moiety becomes located in close proximity to the quencher moiety, leading to efficient interaction between the two moieties. This causes decrease of the fluorescence intensity from the probe compared to the dissociated conformation in which the stem duplex is dissociated and thus the fluorescer moiety is located distal from the quencher moiety. Such conformational change is schematically depicted in FIG. 17.

In order to perform receptor-binding assay, about 50 pmol of each unimolecular probe was mixed with 0, ⅛, and ¼ molar equivalent of streptavidin (0, 6.25, 12.5 pmol, respectively) in 50 μL of pH 8.5 10 mM Tris containing 50 mM KCl, 2.5 mM MgCl₂ and introduced into each well of a 96 well PCR plate (Bio-Rad, CA, USA). Note that ¼ molar equivalent of streptavidine corresponds to 1 molar equivalent of the biotin binding site in streptavidin with respect to the amount of the unmolecular probe used because streptavidin has 4 biotin binding sites. The assay solutions were then incubated for about 30 min at 60° C. The temperature dependence of the fluorescence signal from each well was measured with an iCycler iQ real time PCR machine purchased from Bio-Rad (CA, USA) using the melting profile protocol.

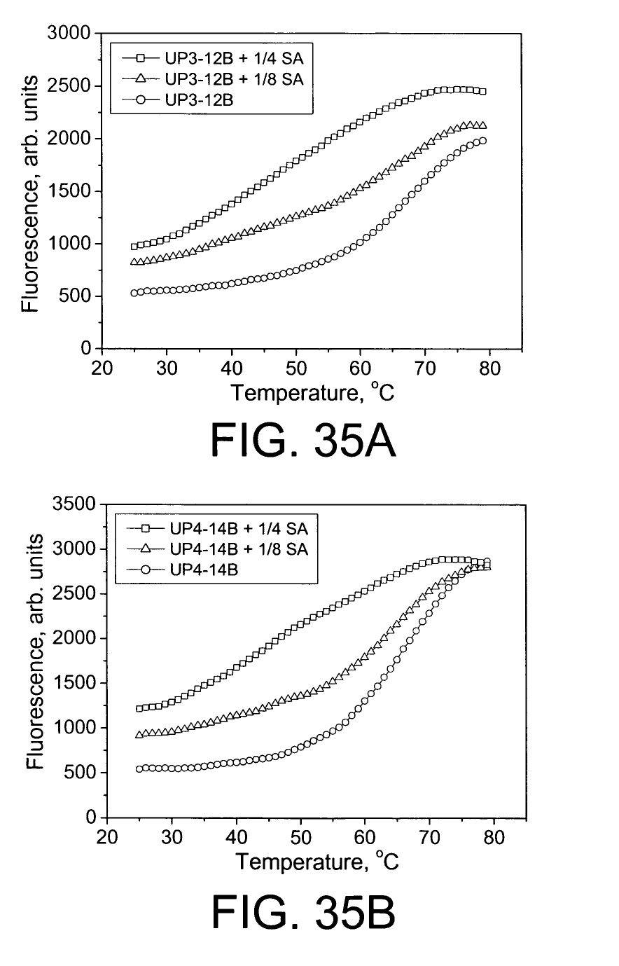

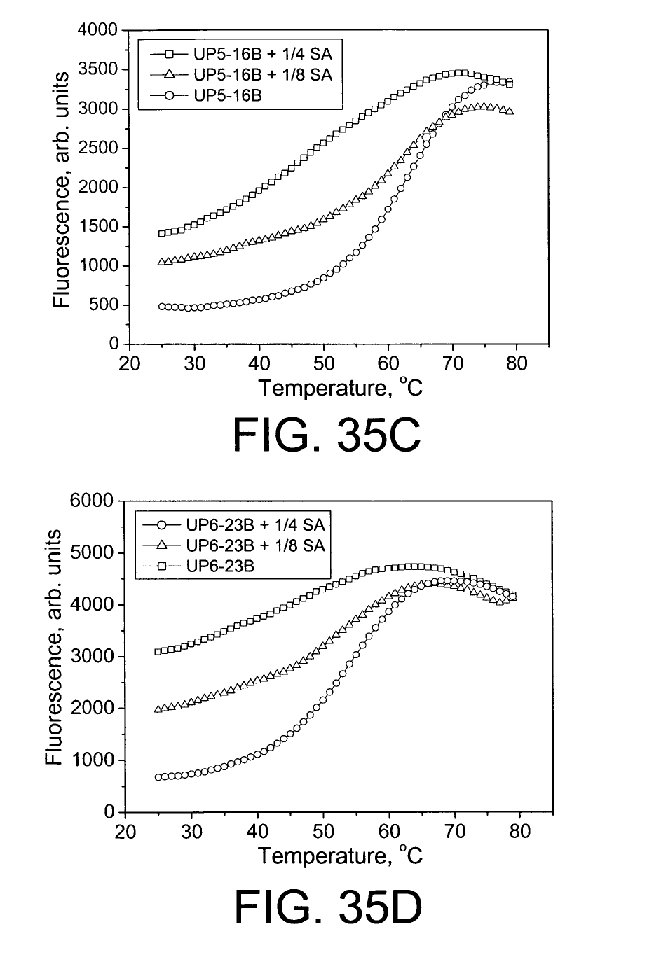

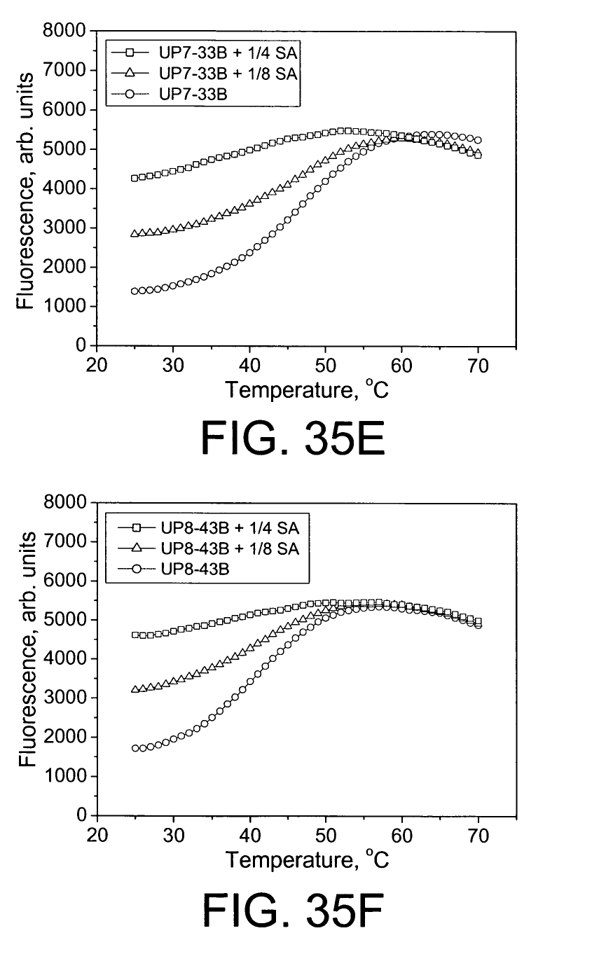

FIG. 35A-F show temperature dependence of the fluorescence signal of each of the six unimolecular probes, UP3-12B (SEQ ID NO: 138), UP4-14B (SEQ ID NO: 139), UP5-16B (SEQ ID NO: 140), UP6-23B (SEQ ID NO: 141), UP7-33B (SEQ ID NO: 142), and UP8-43B (SEQ ID NO: 143), respectively, at different concentrations of the receptor agent, streptavidin. In the absence of the receptor agent (circles in each of FIG. 35A-F), the fluorescence signal stays low at background level in the low temperature region, suggesting that the unimolecular probe is in the stem-loop conformation (“hybridized conformation”), but it shows a sigmoid increase as temperature increases, suggesting that the unimolecular probe changes its conformation to the “dissociated conformation” at a high temperature region. This is a typical shape of the melting profile of the stem loop structure.

Upon addition of the receptor agent, the fluorescence signal increases as a function of the streptavidin concentration, suggesting that binding of the receptor agent (streptavidin) to the probe ligand (biotin) causes conformational switching from the hybridized conformation to the dissociated conformation due to the “nucleic acid hybridization destabilization force” according to the invention. The fluorescence intensity increases accordingly in the presence of the receptor agent. It is noteworthy that the fluorescence intensity is similar in the high temperature region above the melting temperature of the stem duplex regardless of the receptor agent concentration. This suggests that the unimolecular probes have dissociated conformations at the high temperature region due to thermal melting, independent of the receptor agent concentration. In the low temperature region, however, particularly near or below the melting temperature of the stem duplex, the fluorescence intensity is substantially higher at higher receptor agent concentration due to the destabilization effect caused by the binding of the receptor agent to the probe ligand.

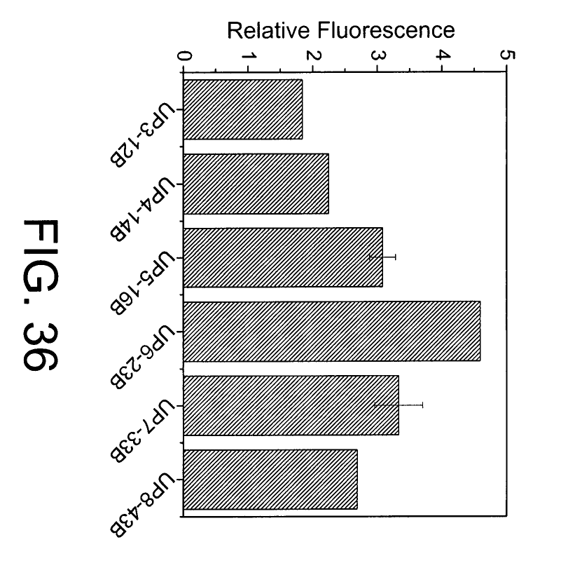

FIG. 36 shows relative increase of the fluorescence intensity measured at 25° C. upon addition of ¼ molar equivalent of streptavidin relative to that with no streptavidin added (i.e., fluorescence intensity at ¼ molar equivalent of streptavidin divided by that without streptavidin). This value can be taken as a figure of merit for the performance of the probe. As shown in FIG. 36, all of the probes tested show large increase of the fluorescence intensity upon addition of the receptor agent, i.e., a factor of about 2 to about 5 increase. The relative increase of the fluorescence intensity is observed to depend on the loop length with the largest value observed when the number of nucleotides in the loop region is 15, i.e., for the case of UP6-23B (SEQ ID NO: 141). This result suggests that performance of the probe can be optimized by adjusting the length of the loop region.

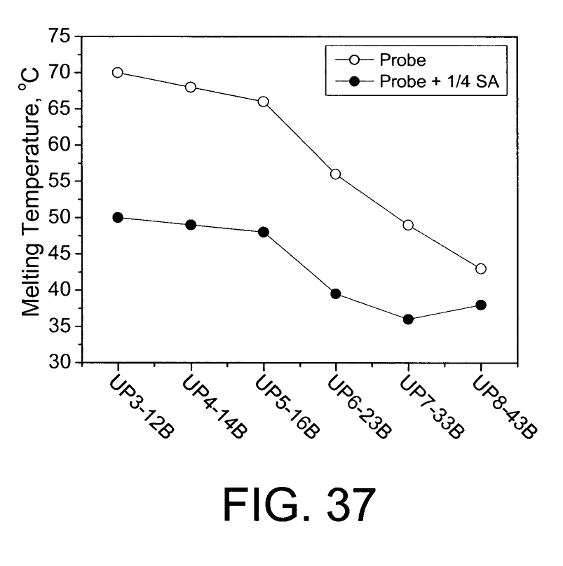

FIG. 37 shows the melting temperature of the unimolecular probes determined from the first derivative of the temperature dependence shown in FIG. 35A-F. The open circles denote the melting temperatures of the probe in the absence of streptavidin and the solid circles denote those in the presence of ¼ equivalent of streptavidin. As expected, the melting temperatures of the probes become lowered by about 5° C. to 20° C. upon addition of the receptor agent according to the invention. The observed decrease of the melting temperature in the presence of the receptor agent suggests that the nucleic acid hybridization destabilization effect derived by the receptor agent binding is sufficient to induce the probe conformation switching.

### Example 12

### Stem Sequence Dependence of the Fluorescence Characteristics of the Non-Competitive Unimolecular Probe Having an Interactive Label Pair Consisting of a Fluorescer and a Quencher

Six non-competitive unimolecular affinity probes having structures similar to the one depicted in FIG. 17 were synthesized and used to examine stem sequence dependence of the fluorescence characteristics of the non-competitive unimolecular probe. The six unimolecular probes, UP6-23B (SEQ ID NO: 141), UP9-23B (SEQ ID NO: 144), UP10-23B (SEQ ID NO: 145), UP11-23B (SEQ ID NO: 146), UP12-23B (SEQ ID NO: 147), and UP13-23B (SEQ ID NO: 148), have the same loop length (i.e., 15 nucleotides) and the same stem hybridization length (i.e., 5 bp stem duplex), but have different stem sequences. All of these unimolecular probes have a biotin as a probe ligand (to detect a target receptor agent, streptavidin) linked to the same position within the 3′ terminal stem region, i.e., to the third nucleotide, thymidine from the 3′ terminus (see FIG. 23A for the structure of the biotin-coupled thymidine). Each unimolecular probe has an interactive label pair consisting of a fluorescer, fluorescein, and a quencher, DABCYL, which are covalently linked to the 5′ and the 3′ terminus by a linker, respectively. The structures of the fluorescein and DABCYL with the linker are shown in FIGS. 23B and 23D, respectively. The unimolecular probes used in this example are also designed to have similar properties to those used in Example 3 and 11.

The assay solutions for the unimolecular probes (50 pmol of each probe with 0, ⅛, and ¼ molar equivalent of streptavidin) were prepared in a 96 well PCR plates by the same procedure described in Example 11. As in Example 11, the temperature dependence of the fluorescence signal was measured with an iCycler iQ real time PCR machine.

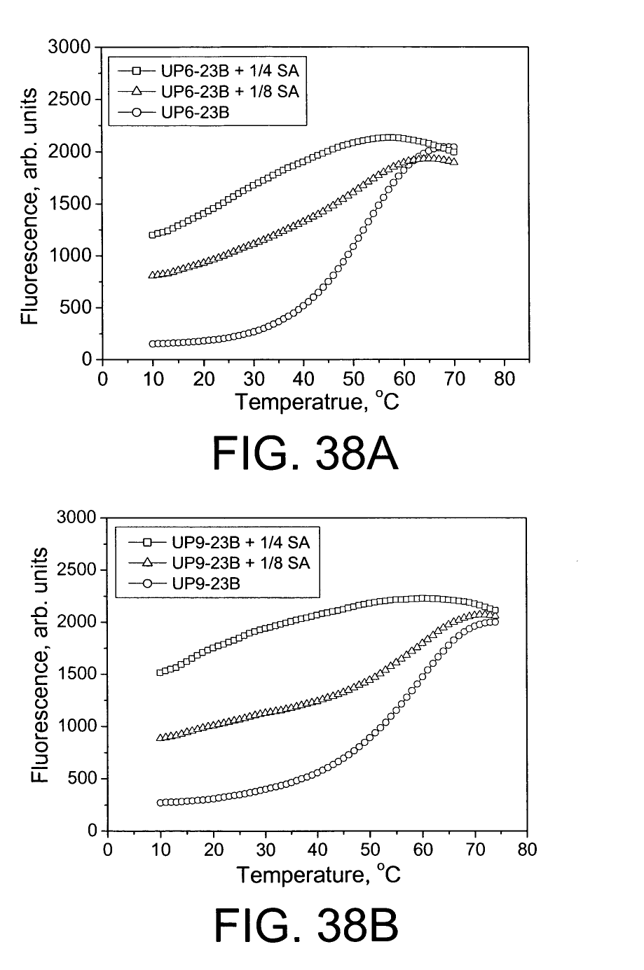

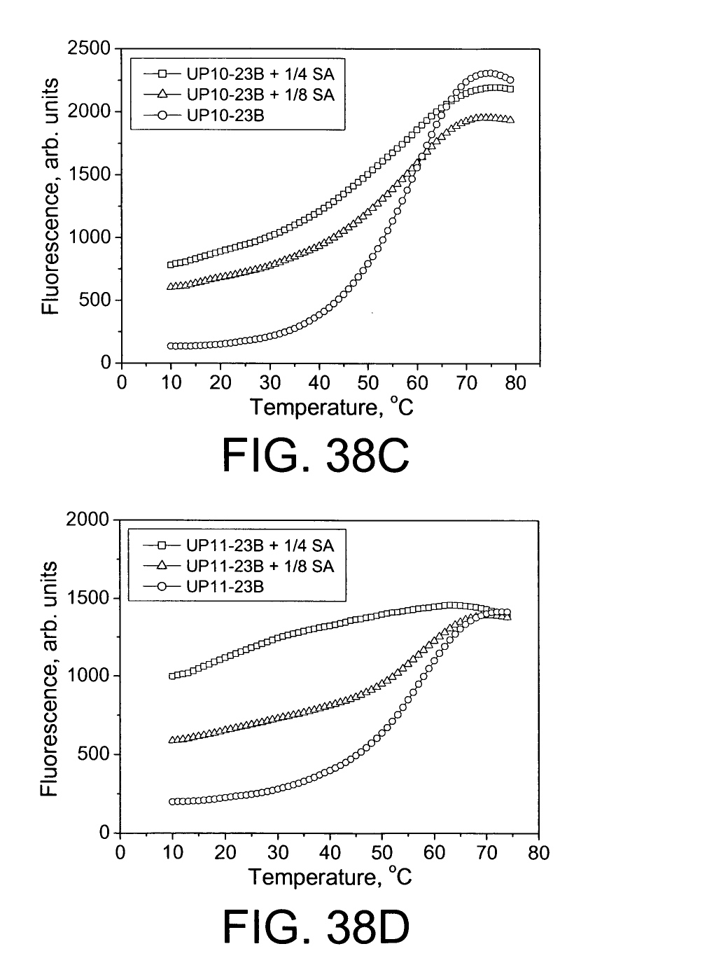

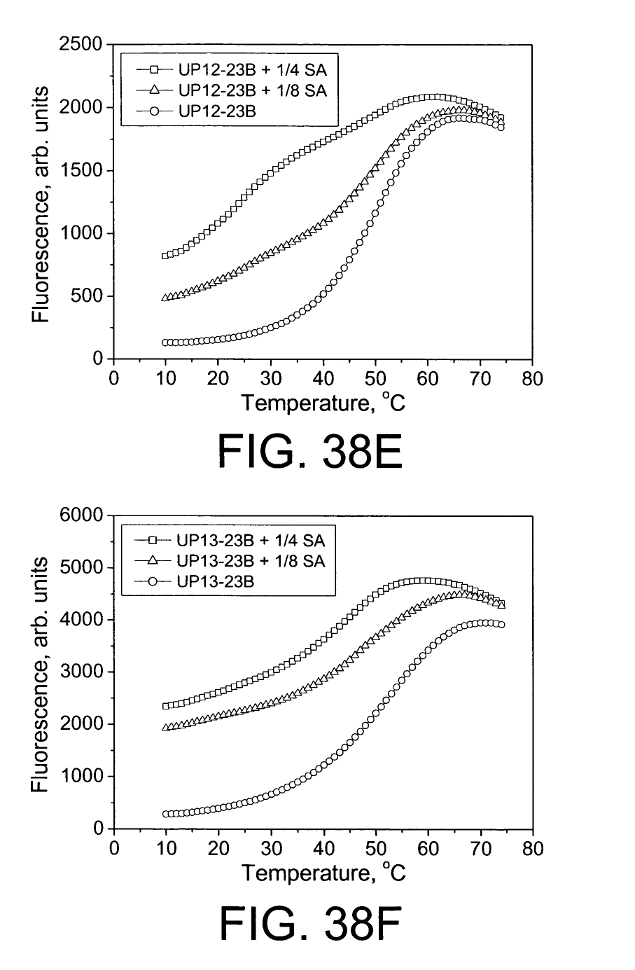

FIG. 38A-F show temperature dependence of the fluorescence signal of each of the six unimolecular probes, UP6-23B (SEQ ID NO: 141), UP9-23B (SEQ ID NO: 144), UP10-23B (SEQ ID NO: 145), UP11-23B (SEQ ID NO: 146), UP12-23B (SEQ ID NO: 147), and UP13-23B (SEQ ID NO: 148), respectively, at different concentrations of the receptor agent, streptavidin. Similar to the results observed for the unimolecular probes used in Example 11 (see FIG. 35A-F), the melting transition from the hybridized conformation (i.e., the stem loop structure) in the low temperature region to the dissociated conformation in the high temperature region was observed for all the probes in the absence of the receptor agent and addition of the receptor agent resulted in substantial increase of the fluorescence intensity as a function of the receptor agent concentration.

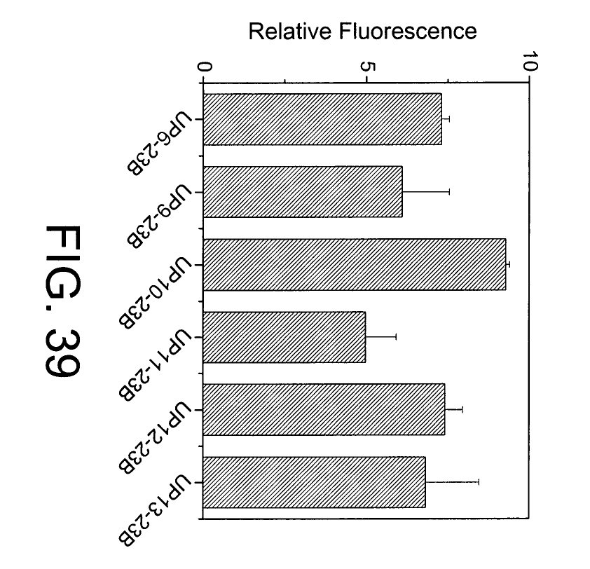

The relative increase of the fluorescence intensity upon addition of ¼ molar equivalent of streptavidin relative to that with no streptavidin added was measured at 25° C., and depicted in FIG. 39. It can be observed in FIG. 39 that the value of the relative increase depends strongly (from about 4 to about 12) on the stem sequences, i.e., the first object and first complement sequences, suggesting that performance of the probe can be optimized by adjusting the stem sequence of the probe.

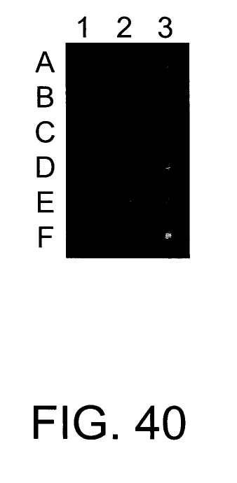

FIG. 40 shows an example of a fluorescence image of a microwell plate with each well containing assay solutions of the following compositions: 50 pmol of UP6-23B (SEQ ID NO: 141) in row A, B and C; 50 pmol of UP10-23B (SEQ ID NO: 145) in row D, E and F; 0, 6.25, and 12.5 pmol streptavidin in column 1, 2 and 3, respectively. Each composition was examined in triplicate as described above. As shown in FIG. 40, systematic increase of the fluorescence intensity as a function of the amount of the receptor agent is observed and it is reproducible.

### Example 13

### Stem Length Dependence of the Fluorescence Characteristics of the Non-Competitive Unimolecular Probe Having an Interactive Label Pair Consisting of a Fluorescer and a Quencher

Two non-competitive unimolecular affinity probes, UP14-21B (SEQ ID NO: 149) and UP1-34B (SEQ ID NO: 127) having 4 and 6 bp stem duplexes, respectively, were synthesized and used to examine stem length dependence of the fluorescence characteristics of the non-competitive unimolecular probe. Same as the unimolecular probes used in Example 11 and 12, UP14-21B (SEQ ID NO: 149) and UP1-34B (SEQ ID NO: 127) have a biotin linked to the third nucleotide, thymidine from the 3′ terminus (see FIG. 23A for the structure of the biotin-coupled thymidine). Each unimolecular probe has an interactive label pair consisting of a fluorescer, fluorescein, and a quencher, DABCYL, which are covalently linked to the 5′ and the 3′ terminus by a linker, respectively. The structures of the fluorescein and DABCYL with the linker are shown in FIGS. 23B and 23D, respectively.

Again, the assay solutions for the above unimolecular probes (50 pmol of each probe with 0, ⅛, and ¼ molar equivalent of streptavidin) were prepared in a 96 well PCR plates by the same procedure described in Example 11, and the temperature dependence of the fluorescence signal was measured with an iCycler iQ real time PCR machine.

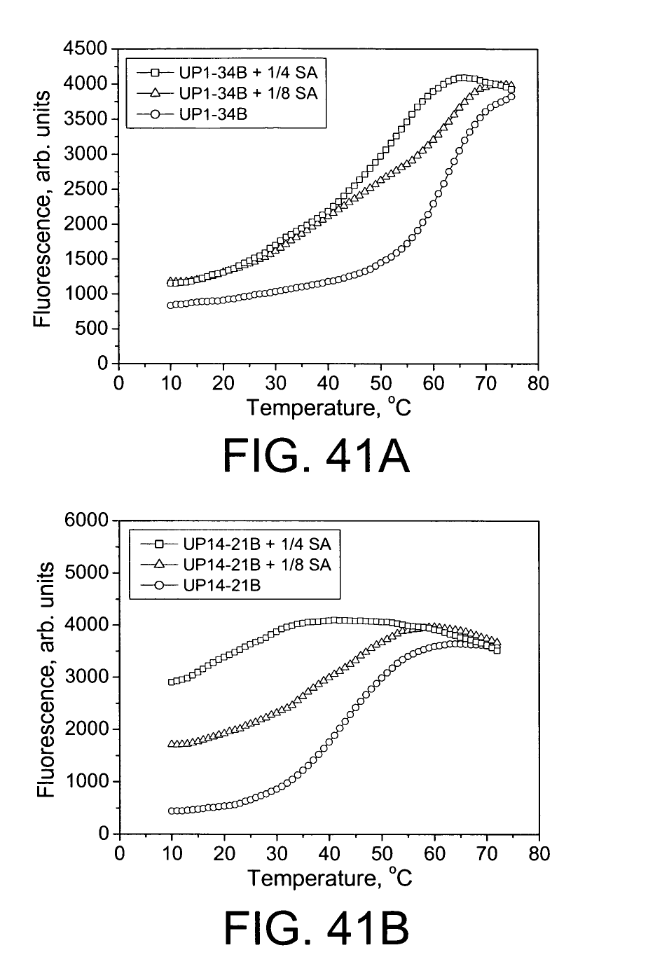

FIGS. 41A and B show temperature dependence of the fluorescence signal of each of the above two unimolecular probes at different streptavidin concentrations. The melting profiles and the response to the addition of the receptor agent, streptavidin are observed to be generally similar to the unimolecular probes having 5 bp stem duplexes (see FIG. 38A-F for comparison) with the melting temperatures being different depending on the stem length (and also the stem sequence and the loop length). The probe UP1-34B having a 6 bp stem duplex structure shows about 20° C. higher melting temperature than that of the probe UP14-21B that has a 4 bp stem duplex structure. The relative increase of the fluorescence signal is observed to be larger for the latter probe having the 4 bp stem duplex structure. This result suggests that the stem length (i.e., the hybridization length of the first object and first complement sequences) in addition to the loop length and the stem sequence as examined in Example 11 and 12 is an important parameter for optimizing the performance and working temperature of the invention probes.

### Example 14

### Loop Sequence Dependence of the Fluorescence Characteristics of the Non-Competitive Unimolecular Probe Having an Interactive Label Pair Consisting of a Fluorescer and a Quencher

Two non-competitive unimolecular affinity probes, UP15-23B (SEQ ID NO: 150) and UP16-23B (SEQ ID NO: 151) having the same stem sequence and loop length as UP6-23B (SEQ ID NO: 141), but with different loop sequences, were synthesized and used to examine loop sequence dependence of the fluorescence characteristics of the non-competitive unimolecular probe. Same as UP6-23B (SEQ ID NO: 141) used in Example 11 and 12, the above two unimolecular probes also have a biotin linked to the same position, the third nucleotide, thymidine from the 3′ terminus, and an interactive label pair consisting of fluorescein and DABCYL covalently linked to the 5′ and the 3′ terminus, respectively. The loop sequences of the above two probes are selected such that each probe has a second stable 4 bp stem duplex conformer between the 5′ terminal region and the loop region, while having a 5 bp stem duplex conformer between the 5′ and 3′ terminal region as the most stable conformer. In contrast, each of the unimolecular probes examined in Example 11 and 12, including UP6-23B (SEQ ID NO: 141) that has the same 5 bp stem duplex sequence and the same loop length as the above two probes, was designed to have only one stem duplex conformer by suitably selecting the loop sequence.

Again, the assay solutions for the above two unimolecular probes (50 pmol of each probe with 0 and ¼ molar equivalent of streptavidin) were prepared in a 96 well PCR plates by the same procedure described in Example 11, and the temperature dependence of the fluorescence signal was measured with an iCycler iQ real time PCR machine.

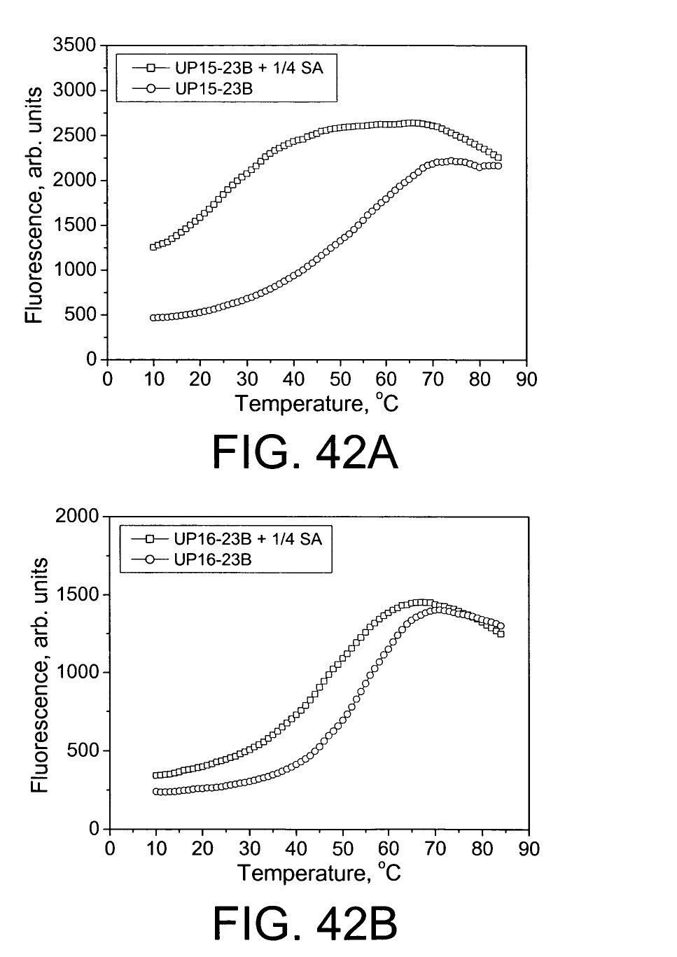

FIGS. 42A and B show temperature dependence of the fluorescence signal of each of the above two unimolecular probes with 0 (circle) and ¼ molar equivalent (rectangular) of streptavidin. The melting profiles and the response to the addition of the receptor agent (streptavidin) are observed to be generally similar to those of the unimolecular probe UP6-23B (SEQ ID NO: 141) that has only one stem duplex conformer (see FIG. 35D for comparison). However, the relative increase of the fluorescence signal upon addition of the receptor agent (streptavidin) is observed to be smaller compared to that of UP6-23B having no secondary stem duplex conformer. For instance, the fluorescence signals of UP15-23B and UP16-23B increase by a factor of about 3 and about 1.5, respectively, upon addition of ¼ equivalent of streptavidin (at 25° C.) while that of UP6-23B increases by a factor of about 5 (see FIG. 37). This result can be readily understood in terms of formation of secondary stem duplex structures of UP15-23B and UP16-23B. Upon binding of the receptor agent to the probe, the primary stem duplex structures become unstable due to the nucleic acid hybridization destabilization effect induced by the receptor agent binding to the probe ligand. This force will cause conformational shift of UP15-23B and UP16-23B to the second stable 4 bp stem duplex structures, while it will cause UP6-23B to shift to the dissociated conformation. Therefore, after the receptor agent binding, the two interacting label moieties are closer to each other in UP15-23B and UP16-23B (due to the secondary stem duplex formation) compared to those in UP6-23B that does not have a secondary stem duplex structure. The fact that UP15-23B shows a larger increase than UP16-23B upon addition of the receptor agent can also be readily understood by the above mechanism. The secondary 4 bp stem duplex of UP15-23B has 4 nucleotides in its loop region, while that of UP16-23B has 13 nucleotides in its loop region. Therefore, the two interacting label moieties are more distal in the secondary stem duplex structure of UP15-23B than that of UP16-23B, resulting in a large increase of the fluorescence signal in the case of UP16-23B upon addition of the receptor agent. The result obtained in this example suggests that selection of the loop sequence with respect to the stem duplex sequence is very important in optimizing the performance of the invention probes. It is preferred that the loop sequence be selected so as to avoid formation of the secondary stem duplex structure(s).

### Example 15

### Probe Ligand Conjugation Position Dependence of the Fluorescence Characteristics of the Non-Competitive Unimolecular Probe Having an Interactive Label Pair Consisting of a Fluorescer and a Quencher

Six non-competitive unimolecular affinity probes having the same sequence in the loop region and the same number of base pairs (5 bp) in the stem duplex, but having a probe ligand (biotin) conjugated to different positions, were synthesized to examine probe ligand conjugation position dependence of the non-competitive unimolecular probe. Among which, three probes, UP10-25B (SEQ ID NO: 152), UP10-23B (SEQ ID NO: 145) and UP10-21B (SEQ ID NO: 153), have a biotin conjugated to the stem duplex region, i.e., conjugated to the 1st, 3rd and 5th nucleotide from the 3′ terminus, respectively. The other three probes, UP10-19B (SEQ ID NO: 154), UP10-13B (SEQ ID NO: 155) and UP10-10B (SEQ ID NO: 156), have a biotin conjugated to the loop region, i.e., conjugated to the 2nd nucleotide from the 3′ end of the loop region, the 8th nucleotide from both the 5′ and 3′ ends of the loop region, and the 5th nucleotide from the 5′ end of the loop region, respectively. Therefore, the former three probes have their probe ligand conjugated to the first object or first complement sequence, but the latter three have their probe ligand outside the first object and first complement sequences. Again, each unimolecular probe has an interactive label pair consisting of fluorescein and DABCYL covalently linked to the 5′ and the 3′ terminus by a linker, respectively.

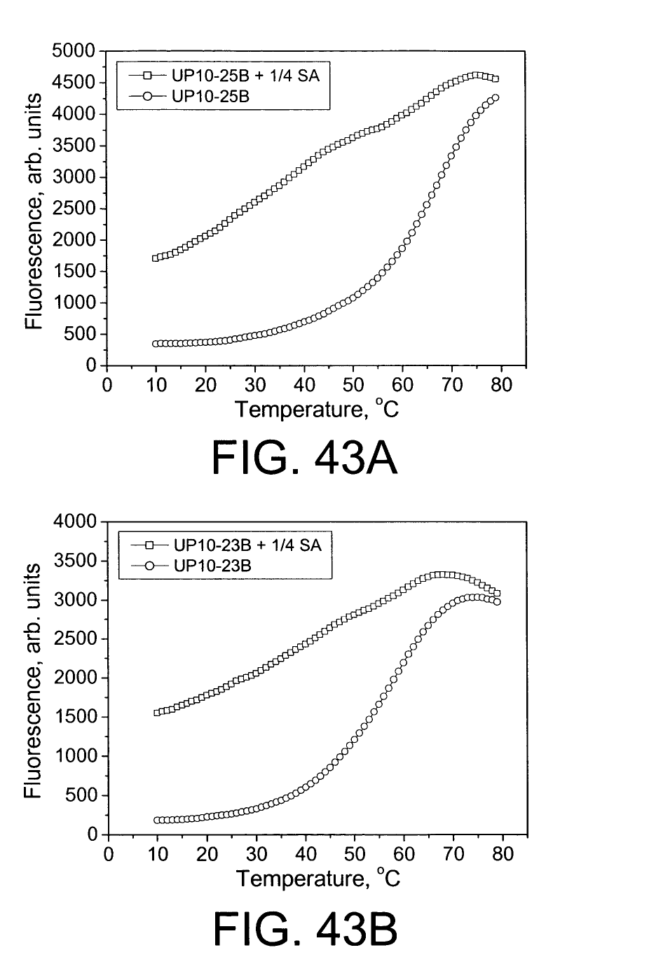

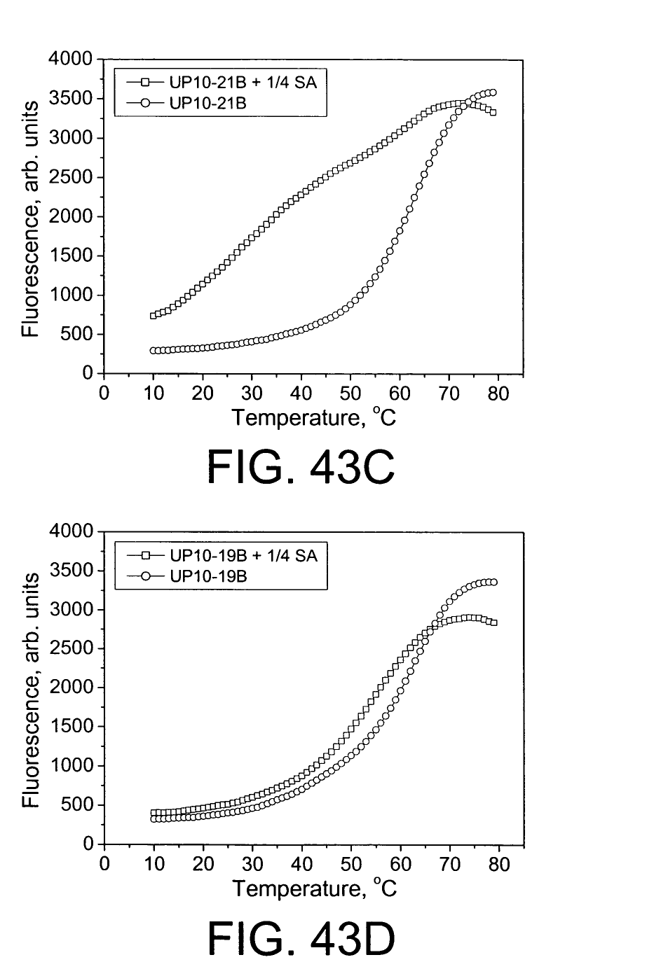

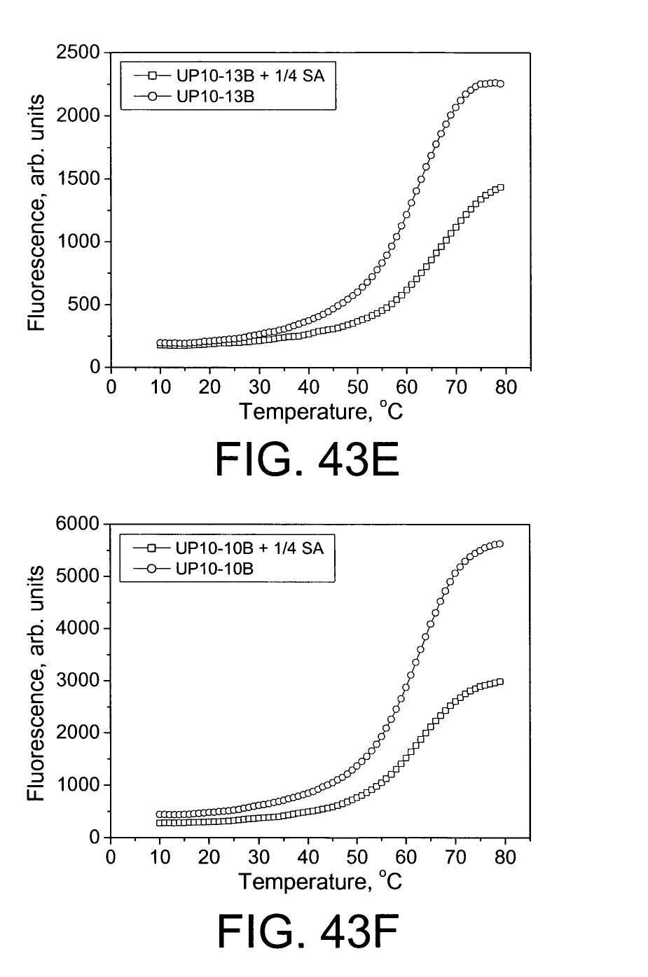

FIG. 43A-F show results obtained with above six unimolecular probes when ¼ molar equivalent of streptavidin relative to the amount of the probe was added as compared to that without addition of streptavidin. The unimolecular probes having the probe ligand conjugated to the stem duplex region (i.e., the first object or first complement sequence) generally show increase of the fluorescence signal upon addition of the receptor agent (streptavidin) with different relative increase depending on the conjugation position (see FIG. 43A-C). However, the other three probes that have the probe ligand conjugated outside the stem duplex region (i.e., in the loop region) do not show significant increase of the fluorescence signal upon addition of the receptor agent (see FIG. 43D-F). Even two of these probes show decrease of the fluorescence signal. These results suggest that the conjugation position of the probe ligand (or recognition element) must be selected appropriately in designing the probes according to the invention.

### Example 16

### Effect of MgCl₂ Concentration in the Melting Profile of the Non-Competitive Unimolecular Probe Having an Interactive Label Pair Consisting of a Fluorescer and a Quencher

Effect of MgCl₂ concentration in the melting profile was examined with the unimolecular probe UP6-23B (SEQ ID NO: 141) having a biotin conjugated to the third nucleotide thymidine from the 3′ terminus and an interactive label pair consisting of fluorescein and DABCYL covalently linked to the 5′ and the 3′ terminus, respectively.

Assay solutions for the above unimolecular probe (50 pmol of each probe with 0 and ¼ molar equivalent of streptavidin) were prepared in a 96 well PCR plates by the same procedure described in Example 11, and the temperature dependence of the fluorescence signal was measured.

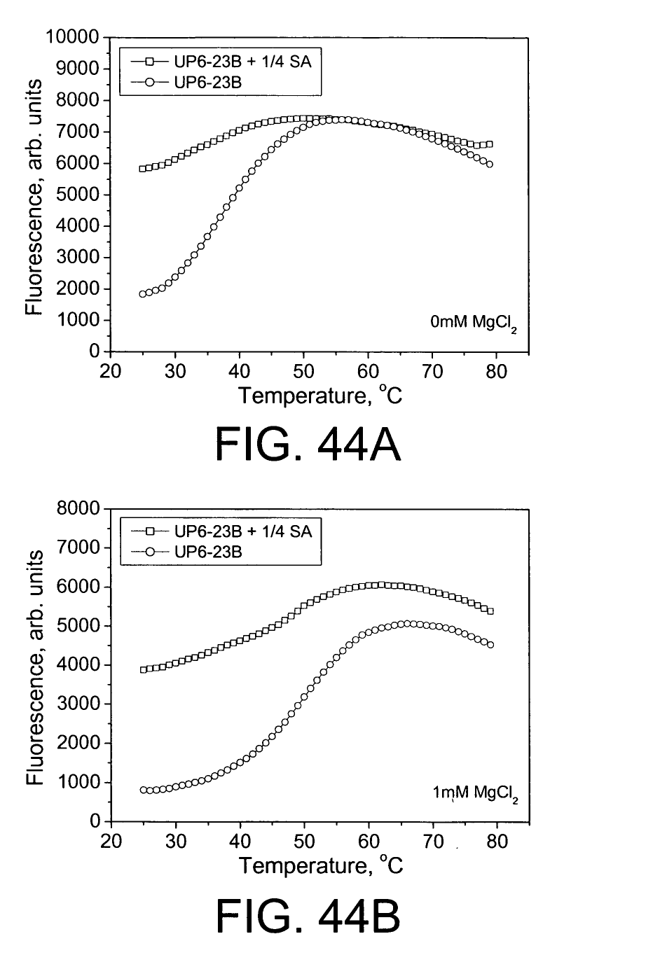

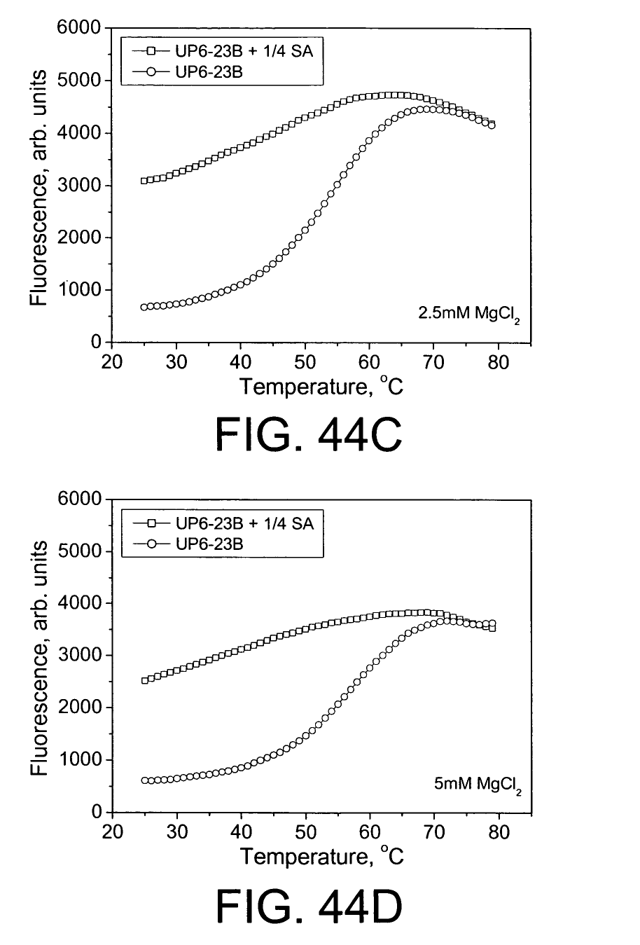

FIG. 44A-D show the results obtained at 0, 1, 2.5 and 5 mM MgCl₂ concentration, respectively. The temperature dependence profiles are observed to be generally similar, but the melting temperature increases as the MgCl₂ concentration increases as expected. In the absence of the receptor agent (circle), the melting temperature increases from about 38° C. at 0 mM MgCl₂ to about 58° C. at 5 mM MgCl₂. The melting temperature in the presence of the receptor agent also increases from about 33° C. at 0 mM MgCl₂ to about 45° C. at 5 mM MgCl₂. This result suggests that control of the concentration of MgCl₂ or the like such as salts or intercalating agents is useful when the probe working temperature needs to be adjusted in accord with the optimum interaction temperature of the target agent with the recognition element.

### Example 17

### pH Dependence of the Fluorescence Characteristics of the Non-Competitive Unimolecular Probe Having an Interactive Label Pair Consisting of a Fluorescer and a Quencher

Effect of pH in the fluorescence characteristics was examined with the unimolecular probe UP6-23B (SEQ ID NO: 141) having a biotin conjugated to the third nucleotide thymidine from the 3′ terminus and an interactive label pair consisting of fluorescein and DABCYL covalently linked to the 5′ and the 3′ terminus, respectively.

Assay solutions for the above unimolecular probe (50 pmol of each probe with 0 and ¼ molar equivalent of streptavidin with total volume including the buffer solution being 50 μL) were prepared in a 96 well PCR plates using the following four buffer solutions: pH 6.0 10 mM MES containing 50 mM KCl, 2.5 mM MgCl₂; pH 7.0 10 mM Tris containing 50 mM KCl, 2.5 mM MgCl₂; pH 8.0 10 mM Tris containing 50 mM KCl, 2.5 mM MgCl₂; and pH 9.0 10 mM Tris containing 50 mM KCl, 2.5 mM MgCl₂. The temperature dependence of the fluorescence signal from each well was measured with an iCycler iQ real time PCR machine.

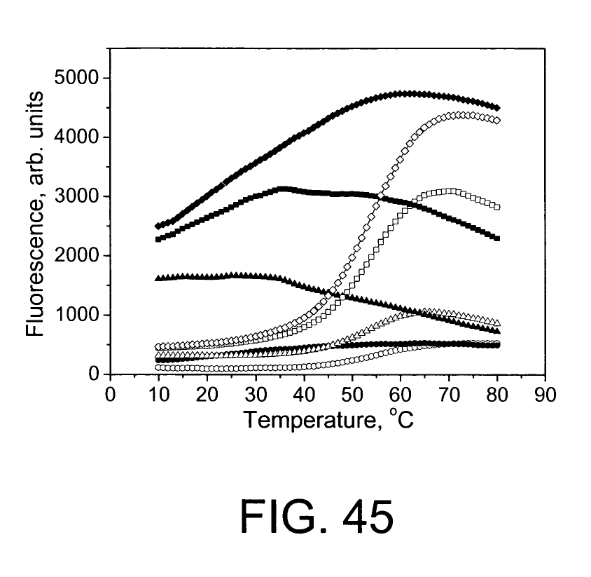

FIG. 45 shows the results obtained at pH 6.0 (circles), 7.0 (triangles), 8.0 (diamonds), and 9.0 (rectangles). The open and solid marks correspond to without and with streptavidin. It is observed that the melting temperature increases with pH as expected, and furthermore the fluorescence intensity increases dramatically depending on the pH. The observed increase of the fluorescence intensity is due to the pH dependent fluorescence characteristics of fluorescein as reported previously. These results suggest that control of pH (in addition to the MgCl₂ concentration) of the assay solution is very useful for optimizing the probe working temperature. The pH dependence of the fluorescence intensity is suggested to be also important in the use of the invention probes. According to the above results, fluorescein used as a fluorescer in this example emits stronger fluorescence at higher pH. Therefore, it will be advantageous to use fluorescene as a fluorescer moity for high pH assays. If assays using the probes of the present invention need to be performed at low pH, it will be desirable to use a different fluorescer that can emit strong fluorescence at low pH.

### Example 18

### Examination of Inhibition of Binding of the Receptor Agent to the Probe Ligand Using a Non-Competitive Unimolecular Probe Having an Interactive Label Pair Consisting of a Fluorescer and a Quencher

Inhibition of the receptor agent binding to the probe ligand was examined using the unimolecular probe UP6-23B (SEQ ID NO: 141) having a biotin conjugated to the third nucleotide thymidine from the 3′ terminus and an interactive label pair consisting of fluorescein and DABCYL covalently linked to the 5′ and the 3′ terminus, respectively.

Assay solutions for the inhibition test were prepared as follows. In each well of a 96 well PCR plate, 50 μL of 10 mM Tris, pH 8.5, 50 mM KCl, 2.5 mM MgCl₂ containing 12.5 pmol of streptavidin and different amount of an inhibitor (biotin) was introduced and incubated for about 30 min at room temperature. About 50 pmol of the probe UP6-23B (SEQ ID NO: 141) was then added to each well and incubated for about 30 min at 60° C. before the fluorescence measurement.

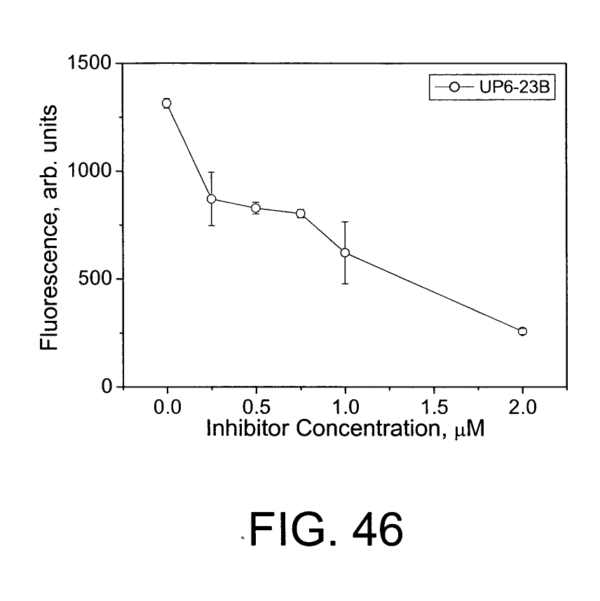

FIG. 46 shows the fluorescence intensity from each well (after subtracting the fluorescence intensity measured for an assay solution not containing streptavidin and the inhibitor) as a function of inhibitor concentration. The fluorescence intensity decreases with increasing inhibitor concentration, indicating that binding of the receptor agent (streptavidin) to the probe ligand (biotin conjugated to the probe) is inhibited by the inhibitor (free biotin). This result suggests that the probes of the present invention can be used for screens to find certain inhibitors that blocks or suppresses interaction of the target agent and the recognition element.

### Example 19

### Assay for Detecting Streptavidin Using Non-Competitive Bimolecular Probes with an Interactive Label Pair Consisting of a Fluorescer and a Quencher

Two bimolecular affinity probes having their structures similar to that depicted in FIG. 21 were constructed and examined for their response to the concentration of the target receptor agent. The first bimolecular probe consists of CM4* (SEQ ID NO: 157) as a first complement sequence (or a first object sequence) and CM5-11B (SEQ ID NO: 158) as a first object sequence (or a first complement sequence). The second bimolecular probe consists of CM4* (SEQ ID NO: 157) as a first complement sequence (or a first object sequence) and CM6-11B (SEQ ID NO: 159) as a first object sequence (or a first complement sequence). CM4* (SEQ ID NO: 157) has 5′and 3′ arm sequences for formation of a 5 bp stem duplex structure and an interactive label pair consisting of fluorescein and DABCYL covalently linked to the 5′ and the 3′ terminus, respectively. CM5-1B (SEQ ID NO: 158) and CM6-1B (SEQ ID NO: 159) are 15mer and 13mer oligonucleotides having a biotin as a probe ligand conjugated to the 11th and nucleotide, thymidine from the 5′ terminus. In above probe compositions, CM5-11B (SEQ ID NO: 158) and CM6-11B (SEQ ID NO: 159) act as first object sequences (or first complement sequences) and all or a part of the loop region of CM4* (SEQ ID NO: 157) acts as a first complement sequence (or a first object sequence).

As depicted schematically in FIG. 21, in the absence of the target receptor agent (streptavidin), the bimolecular probes described above are designed to favor the hybridized conformation depicted in FIG. 21A. However, upon binding of the target receptor agent, the bimolecular probes shift their conformation to the dissociated conformation as depicted in FIG. 21B. In the hybridized conformation (i.e., in the absence of the target receptor agent), the two interacting label moieties in CM4* are distal to each other, and thus interaction between the two label moieties becomes weaker or blocked, resulting in emission of strong fluorescence signal. In the dissociated conformation (i.e., in the presence of the target receptor agent), the two molecules become dissociated and thus the two label moieties in CM4* are in close proximity to each other since the stem duplex structure is formed. This results in stronger interaction between the two label moieties, leading to quenching of the fluorescence signal. Therefore, it is expected that fluorescence signal of the bimolecular probes described above decreases as the target receptor agent (streptavidin) concentration increases. This was examined in this example.

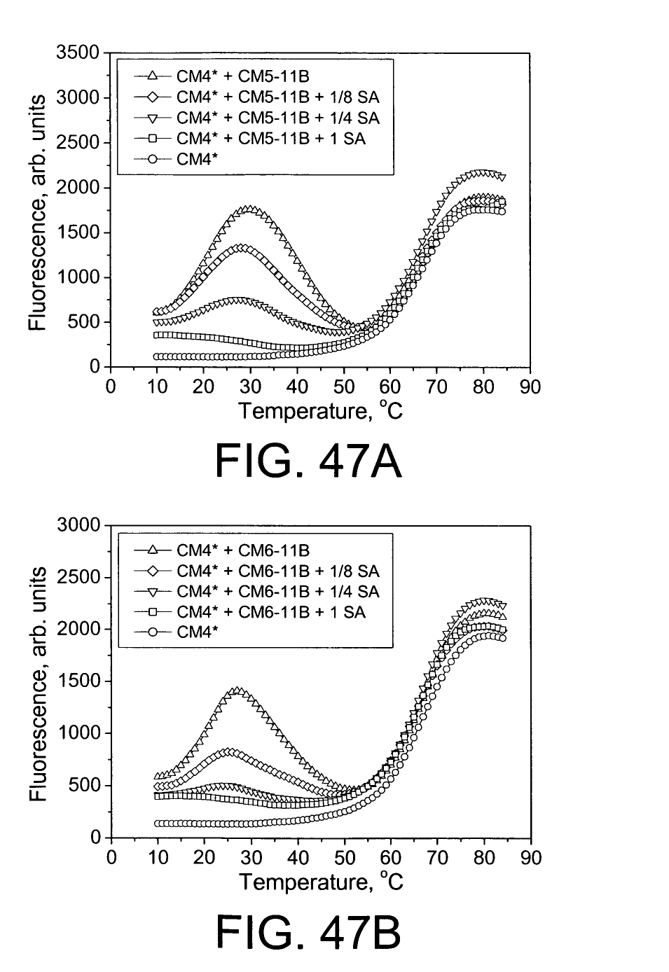

FIGS. 47A and B show fluorescence signals observed for the two bimolecular probes, CM4*+CM5-11B and CM4*+CM6-11B, respectively, as a function of both the concentration of streptavidin and temperature. In each well of a 96 well plate, 50 pmol each of CM4* and CM5-11B (or CM6-11B) and 0, ⅛, ¼ or 1 molar equivalent of streptavidin was mixed in 50 μL of pH 8.5 10 mM Tris containing 50 mM KCl, 2.5 mM MgCl₂. Assay solution containing 50 pmol of CM4* only in 50 μL of the Tris buffer was also sampled into a separate well as a control to observe the background signal. As shown in FIGS. 47A and B, the bimolecular probes show strong fluorescence signal in the absence of the target receptor agent (see the temperature range between about 20 to about 40° C.), indicating that the first object and first complement sequences are hybridized and thus the stem duplex of CM4* is dissociated to make the two label moieties distal to each other. Upon addition of the target receptor agent (streptavidin), the fluorescence signal decreases, indicating that hybridization between the first object and first complement sequences becomes unstable and thus they dissociates from each other. Such dissociation results in formation of the stem duplex structure of CM4*, leading to efficient interaction of the two label moieties, i.e., quenching of the fluorescence as observed.

### Example 20

### Assay for Detecting Streptavidin Using a Non-Competitive Bimolecular Probe Similar to Those Examined in Example 19

A bimolecular affinity probe similar to those examined in Example 19 was constructed and examined for its response to the concentration of the target receptor agent. The bimolecular probe consists of UP10-13B (SEQ ID NO: 155) as a first object sequence (or a first complement sequence ) and CM5 (SEQ ID NO: 160) as a first complement sequence (or a first object sequence). UP10-13B (SEQ ID NO: 155) has a biotin as a probe ligand conjugated to the loop region at the 13th nucleotide from the 5′ terminus and an interactive label pair consisting of fluorescein and DABCYL covalently linked to the 5′ and the 3′ terminus, respectively. CM5 is a 15mer oligonucleotide having a sequence complementary to the loop region of UP10-13B. Similar to the bimolecular probes examined in Example 19, CM5 act as a first complement sequence (or a first object sequences) and the loop regions of UP10-13B acts as a first object sequence (or a first complement sequence).

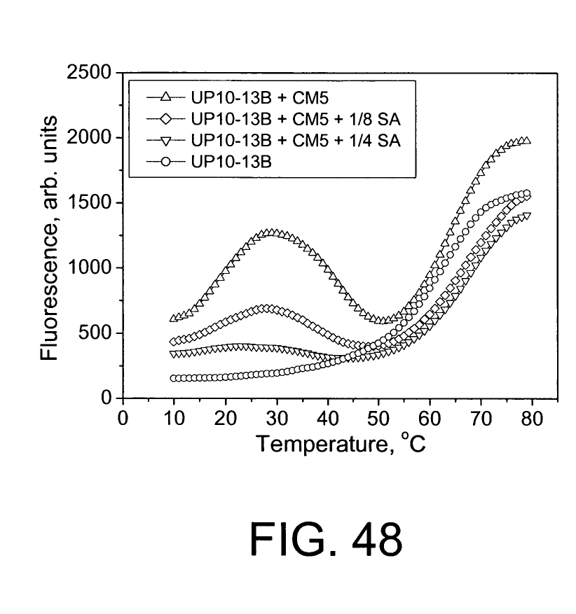

The bimolecular probe constructed in this example are similar in its structure to those examined in Example 19, except that the probe ligand, biotin, is conjugated to the oligonucleotide having a stem loop structure with an interactive label pair in this example and it is conjugated to the linear oligonucleotide without label in Example 19. Assay solutions were prepared similar to Example 19 (i.e., 50 pmol each of UP10-13B and CM5 with 0, ⅛ and ¼ molar equivalent of streptavidin), and the fluorescence signal was measured. The results are presented in FIG. 48. As expected, the response of the bimolecular probe to addition of the target receptor agent is similar to those observed for the bimolecular probes examined in Example 19. It is noteworthy that UP10-13B was examined as a unimolecular probe in Example 15 but it did not work properly as a unimolecular probe. This was attributed in Example 15 to the fact that UP10-13B has its probe ligand conjugated to the loop region (i.e., not within the first object or first complement sequence). However, using the same oligonucleotide UP10-13B in a bimolecular format with an additional oligonucleotide component it was possible to generate the desired change in the fluoresecne signal as a function of the target concentration.

### Example 21

### Assay for Detecting Kinase Activity and its Inhibition Using a Non-Competitive Unimolecular Probe Having Having an Interactive Label Pair Consisting of a Fluorescer and a Quencher

Preparation of a Kinase Probe

A kinase probe was prepared by cross-linking a pre-probe having a reaction group with a peptide using a cross-linker. Two kinds of pre-probes were prepared, which have common backbone structure, a stem-loop structured 25mer oligonucleotide having a fluorescer and a quencher conjucated to 5′ and 3′ termius, respectively. UP10-23C (SEQ ID NO: 161) contains a carboxyl residue covalently linked to the third nucleotide thymidine from the 3′ terminus in the 3′ stem sequence. UP10-23A (SEQ ID NO: 162) contains a primary amine residue at the same site of the stem (see FIGS. 24A and B for the structure of the carboxyl and amine group-coupled thymidine). The choice of pre-probe depends on the reaction group(s) included in oligonucleotide and peptide. When fluorescein is used as a fluorescer, UP10-23A, one having a primary amine, is more preferred as used in this example. The cross-linker used is MBS (m-Maleimidobenzoyl-N-hydroxysuccinimide ester). Since MBS is a NHS-ester-maleimide heterobifunctional cross-linker, it is used for cross-linking two molecules together, first to a primary amine group in one molecule and then to a sulfhydryl group in another molecule. The peptide sequence used in this example was KRTLRRC (SEQ ID NO: 163). The peptide KRTLRR (SEQ ID NO: 164) is known as the recognition sequence of PKC (protein kinase C). PKC phosphorylates the hydroxyl group at the side chain of threonine. A cysteine residue was introduced at the C terminus to provide a sulfhydryl group for conjugation to maleimide ring of the cross-linker.

About 20-fold molar excess of MBS dissolved in DMSO was added dropwise to 1 mM UP10-23A pre-probe solution with rapid mixing and incubated for 30 min at room temperature. By applying the reaction mixture to Sephadex G-25 column, desalted maleimide-activated pre-probe was obtained. About 40-fold molar excess of peptide having the sequence KRTLRRC was then added to the maleimide-activated pre-probe solution and incubated for 30 min at room temperature. After applying the reaction mixture to Sephadex G-25 again, peptide-conjugated probe was purified using Acme HPLC System from Younglin Instrument Co. (Anyang, Korea) using a 10 mm dia×250 mm, C18 HPLC column from Supelco (PA, USA). The product thus prepared is the PKC probe UP10-23PKC (SEQ ID NO: 165) that has a PKC recognition site sequence (SEQ ID NO: 164) conjugated to the third nucleotide from the 3′ terminus and an interacting label pair consisting of fluorescein and DABCYL conjugated to the 5′ and 3′ terminus, respectively. The peptide KRpTLRRC (SEQ ID NO: 166) having a phospholylated threonine Rp was conjugated to the same UP10-23A pre-probe to prepare the PKC control probe, UP10-23PKCp (SEQ ID NO: 167) (see FIGS. 24C and D for the structure of peptide-coupled thymidine).

Kinase Assay

The composition of PKC assay buffer was pH 7.4 20 mM HEPES buffer containing 10 mM MgCl₂, 1 mM ATP, and 1 mM CaCl₂. To detect enzyme activity, 50 pmol of the PKC probe was reacted with 50 mU of PKC for 1 hr at 30° C. in 50 μL of the HEPES buffer. (One unit of PKC is defined as the amount of enzyme required to transfer 1 nmol of phosphate per minute at 30° C. using Type III-S histone protein as the substrate in the presence of 0.6 mg/ml phosphatidylserine and 1.7 mM CaCl₂.) The reaction was terminated by adding EDTA at the final concentration of 10 mM. About 25 pmole of anti-phosphothreonine antibody was added to the reaction mixture, and the fluorescence signal of the sample was detected using an iCycler iQ real time PCR machine. Each of the PKC probe and the PKC control probe were mixed with anti-phosphothreonine antibody without the kinase reaction, as negative and positive controls. To examine the relation between the inhibitor concentration and the PKC activity, 0-90 μM staurosporin, known as a potent PKC inhibitor, was added to the kinase solution and pre-incubated for 5 minutes before the kinase reaction.

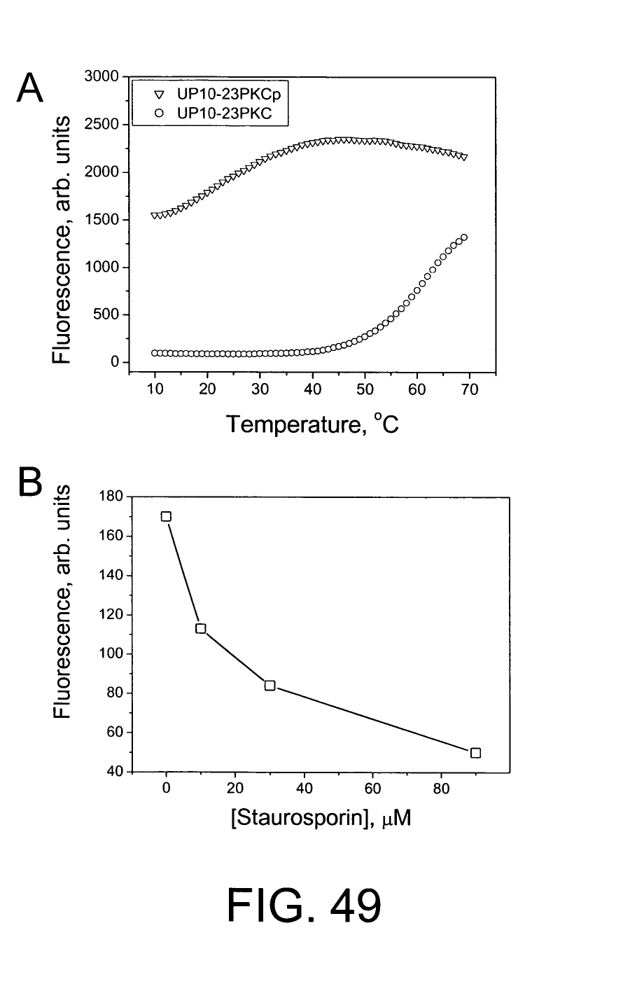

The PKC control probe UP10-23PKCp having pre-phosphorylated threonine showed a larger fluorescence increase when the antibody was added as a destabilizing agent. As shown in FIG. 49A, fluorescence intensity observed from the PKC control probe (50 pmol) in the presence of anti-phosphothreonine antibody (25 pmol) was about 10 fold larger compared to the un-phosphothreonine PKC probe UP10-23PKC in the presence of the same amount of anti-phosphothreonine antibody. This result suggests that the antibody acts as an efficient destabilizing agent that can bind to the phosphorylated peptide sequence included in the PKC control probe and thus destabilize the stem duplex conformation sufficiently according to the invention. FIG. 49A shows results obtained with the kinase reaction described above in the absence and presence of a PKC inhibitor staurosporin. As shown, a substantial increase of the fulorescence signal with respect to that measured with un-reacted PKC probe is observed in the absence of the inhibitor. This result indicates that the PKC recognition site sequence included in the PKC probe as a recognition element was efficiently phosphorylated by the action of PKC and also the anti-phosphothreonine antibody worked as an efficient destabilizing agent according to the invention. In the presence of the inhibitor, the fluorescence signal decreases as the inhibitor concentration increases, indicating that the kinase activity of PKC was inhibited in a concentration dependent manner. The results of this example demonstrate that the reaction probes such as kinase probes or protease probes according to the present invention can be used effectively for screening inhibitors of diverse target reaction inducing agents.

Materials and Methods

The following materials and methods can be used as needed to use the present target detection system.

1) ATP Assay

Determination of ATP using bioluminescence is a well established technique. It uses the ATP dependency of the light emitting luciferase catalyzed oxidation of luciferin for the measurement of extremely low concentrations of ATP (De Luca, M. and McElroy, W. D., (1978)). The working range of the commercial ATP Assay Kits is between 1 nmol to 1 fmol ATP (or between 10⁻⁶ and 10⁻¹¹ M ATP). See product literatures from Roche Molecular Biochemicals, ATP Bioluminescence Assay Kit CLS II, as revised July, 1999; and CalBiochem, ATP Assay Kit Cat. No. 119108, as revised Jan. 26, 1999.

2) Pyrophosphate Assay

Pyrophosphate (PPi) is hydrolyzed to two molecules of inorganic phosphate (Pi) by the reaction catalized by inorganic pyrophosphatase. In the presence of Pi, maltose phosphorylase converts maltose to glucose 1-phosphate and glucose. Then, glucose oxidase converts the glucose to gluconolactone and H₂O₂. Finally, with horseradish peroxidase (HRP) as a catalyst, the H₂O₂ reacts with the Amplex Red reagent (10-acetyl-3,7-dihydroxyphenoxazine) to generate resorufin, which has absorption and fluorescent emission maxima of approximately 563 nm and 587 nm, respectively (Zhou, M. et al., (1997); Hu, G. R. et al., (1997)). This assay can detect as little as 0.2 μM PPi in 100 μl reactions (20 pmol PPi). See product literature from Molecular Probes, PiPer™ Phosphate Assay Kit No. P-22061 as revised on Jul. 17, 2002.

In another scheme, in the presence of Pi, a substrate 2-amino-6-mercapto-7-methylpurine nucleoside phosphorylase (MESG) is converted enzymatically by purine nucleoside phosphorylase (PNP) to ribose 1-phosphate and 2-amino-6-mercapto-7-methylpurine (Webb, M. R., (1992)). Enzymatic conversion of MESG results in a shift in absorbance maximum from 330 nm for the substrate to 360 nm for the product. This assay can detect as little as 1 nmol PPi in 1 ml reactions. See product literature from Molecular Probes, EnzChek Pyrophosphate Assay Kit No. E-6646 as revised on Jun. 21, 2002.

All references disclosed herein are incorporated by reference. The following references are specifically incorporated by reference.
Hirschfeld, T. E., Fluorescent immunoassay employing optical fiber in capillary tube. U.S. Pat. No. 4,447,546 (1984).
Henderson, D. R., Methods for protein binding enzyme complementation assays. U.S. Pat. No. 4,708,929 (1987).
Hampar, B., Showalter, S. D. and Zweig, M., Enzyme-linked immunosorbent assay (ELISA) for determining anti-bodies against herpes simplex virus (HSV) types 1 and 2 in human sera. U.S. Pat. No. 4,764,459 (1988).
Shinbo, T., Yamaguchi, T. and Tachibana, K., Crown ether compound and separating agent, U.S. Pat. No. 4,942,149 (1990).
Dattagupta, N., Solution-phase single hybridization assay for detecting polynucleotide sequences. U.S. Pat. No. 4,968,602 (1990).
Caskey, C. T. and Gibbs, R. A. L., Mutation detection by competitive oligonucleotide priming. U.S. Pat. No. 5,578,458 (1996).
Chehab, F. F., DNA detection by color complementation. U.S. Pat. No. 5,489,507 (1996).
Lucas, J. N., Straume, T. and Bogen, K. T., Detection and isolation of nucleic acid sequences using competitive hybridization probes. U.S. Pat. No. 5,616,465 (1997).
Ebersole, R. C., Moran, J. R. and Ward, M. D., Assay method for biological target complexes on the surface of a biosensor. U.S. Pat. No. 5,658,732 (1997).
Kennedy, C. B., Microfluidic devices and systems. U.S. Pat. No. 5,876,675 (1999).
Parce, J. W., Knapp, M. R., Chow, C. Y. H. and Bousse, L., Controlled fluid transport in microfabricated polymeric substrates. U.S. Pat. No. 5,885,470 (1999).
Parce, J. W., Kopf-Sill, A. R. and Bousse, L. J., High throughput screening assay systems in microscale fluidic devices. U.S. Pat. No. 5,942,443 (1999).
Tyagi, S., Kramer, F. R., and Lizardi, P. M. Detectably labeled dual conformation oligonucleotide probes, assays and kits. U.S. Pat. No. 5,925,517 (1999).
Leiner, M. J. P., He, H. and Boila-Gockel, A., Method of determining an alkali ion. U.S. Pat. No. 5,952,491 (1999).
Taremi, S. S. and Prosise, W. W., Surface plasmon resonance based enzymatic assay. U.S. Pat. No. 5,981,167 (1999).
Lucas, J. N., Straume, T. and Bogen. K. T., Detection and isolation of nucleic acid sequences using a bifunctional hybridization probe. U.S. Pat. No. 6,027,879 (2000).
Whitten, D. G., McBranch, D. W., Jones, R. and Bergstedt, T. S., Improvements to the fluorescent polymer-QTL approach to biosensing. PCT Pub. No. WO 01/85997 A1 (2001).
Aizawa, M., Biosensors in Introduction to Molecular Electronics, Eds Petty, M. C., Bryce, M. R. and Bloor, D., pp. 295-314 (Edward Arnold, London, 1995).
Barrie, K. A., Perez, E. E., Lamers, S. L., Farmerie, W. G., Dunn, B. M., Sleasman, J. W. and Goodenow, M. M., Natural variation in HIV-1 protease, Gag p7 and p6, and protease cleavage sites within gag/pol polyproteins: amino acid substitutions in the absence of protease inhibitors in mothers and children infected by human immunodeficiency virus type I. Virology, 219:407-416 (1996).
Bosworth, N. and Towers, P., Scintillation proximity assay. Nature 341:167-8 (1989).
Brown, T. and Dorcas, J. S., Modem machine-aided methods of oligonucleotide synthesis. In Oligonucleotides and Analogues a Practical Approach. Ed. F. Eckstein (IRL Press Oxford UK, 1995).
Carter, R. M., Mekalanos, J. J., Jacobs, M. B., Aubrano, G. J. and Guilbault, G. G., Quartz crystal microbalance detection of Vibrio cholerae O139 serotype. J. Immunol. Methods, 187:121-5 (1995).
Caruthers, M. H., Gene synthesis machinery: DNA chemistry and its uses. Science 230:281-285 (1985).
Chen, L., McBranch, D. W., Wang, H. L., Helgeson, R., Wudl, F. and Whitten, D. G., Highly sensitive biological and chemical sensors based on reversible fluorescence quenching in a conjugated polymer. Proc. Natl. Acad. Sci. 96:12287-12292 (1999).
Cohen, C., Chin-Dixon, E., Jeong, S., and Nikiforov, T., A microchip-based enzyme assay for protein kinase A. Anal. Biochem. 273:89-97 (1999).
Comoglio, S. and Celda, F., An immuno-enzymatic assay of cortisol using E. coli β-glactosidase as label. J. Immunol. Methods, 10:161-170 (1976).
De Luca, M. and McElroy, W. D., Methods Enzymol. 57:3-15 (1978).
Dempsey, L. A., Brunn, G. J. and Platt, J. L., Heparanase, a potential regulator of cell-matrix interactions. Trends Biochem. Sci. 25:349-351 (2000).
Egholm, M., Buchardt, O., Christensen, L., Behrens, C., Freier, S. M., Driver, D. A., Berg, R. H., Kim, S. K., Norden, B. and Nielsen, P. E., PNA hybridizes to complementary oligonucleotides obeying the Watson-crick hydrogen-bonding rules. Nature, 365:566-568 (1993).
Gluzman, I. Y., Francis, S. E., Oksman, A., Smith, C. E., Duffin, K. L. and Goldberg, D. E., Order and specificity of the Plasmodium falciparum hemoglobin degradation pathway. J. Clin. Invest. 94:1602 (1994).
Gonnelli, M., Gabellieri, E., Montagnoli, G. and Felicioli, R., Complementing S-peptide as modulator in enzyme immonoassay. Biochem. Biophys. Res. Comm. 102:917-923 (1981).
Grakoui, A., Levis, R., Raju, R., Huang, H. V. and Rice, C. M., A cis-acting mutation in the Sindbis virus junction region which affects subgenomic RNA synthesis. J. of Virol., 67:2832 (1993).
Griffin, T. J., Hall, J. G., Prudent, J. R. and Smith, L. M., Direct genetic analysis by matrix-assisted laser desorption/ionization mass spectrometry. Proc. Natl. Acad. Sci., 96:6301-6306 (1 999).
Hall, M. R. T. and Gibson, W., Virology, 227:160 (1997).
Houghtem, R. A., General method for the rapid solid-phase synthesis of large numbers of peptides: specificity of antigen-antibody interaction at the level of individual amino acids. Proc. Natl. Acad. Sci. 82:5131-5 (1985).
Hu, G. R., Harrop, P., Warlow, R. S., Gacis, M. L. and Walls, R. S., High resolution autoantibody detection by optimized protein G-horseradish peroxidase immunostaining in a western blotting assay. J. Immunol. Methods, 202:113-121 (1997).
Jeong, S. and Nikiforov, T. T., Kinase assay based on thiophosphorylation and biotinylation. BioTechniques, 27:1232-1238 (1999).
Killard, A. J., Smyth, M. R. Grennan, K., Micheli, L. and Palleschi, G., Rapid antibody biosensor assays for environmental analysis. Biochem. Soc. Trans. 28, 81-84 (2000).
Knorre, D. G., Vlassov, V. V., Zarytova, V. F., Lebedev, A. V., and Federova, O. S. in Design and targeted reactions of oligonucleotide derivatives, (CRC Press, Boca Raton, Ann Arbor, London, Tokyo, 1994).
Kolykholov, A. A. et al., J. of Virol., 68:7525 (1994).
Lakowicz, J. R., Topics in Fluorescence Spectroscopy, Vol. 4: Probe Design and Chemical Sensing, pp. 133-134 (Plenum Press, New York and London, 1994).
Leiner, M. J. P. and Hartmann, P., Theory and practice in optical pH sensing. Sensors and Actuators B, 11:281-289 (1993).
Liedberg, B., Nylander, C. and Lundstrom, I., Biosensing with surface plasmon resonance—how it all started. Biosens. Bioelectron., 10:i-ix (1995).
Moon, R. P., Tyas, L., Certa, U., Rupp, K., Bur, D., Jacquet, C., Matile, H., Loetscher, H., Grueninger-Leitch, F., Kay, J., Dunn, B. M., Berry, C. and Ridley, R. G., Expression and characterisation of plasmepsin I from Plasmodium falciparum. Eur. J. Biochem., 244:552-560 (1997).
Okada, Y., Yamada, S., Toyoshima, M., Dong, J., Nakajima, M. and Sugahara, K., Structural recognition by recombinant human heparanase that plays critical roles in tumor metastasis: Hierarchical sulfate groups with differential effects and the essential target disulfated trisaccharide sequence. J. Biol. Chem. in print (2002).
Orum, H., Nielsen, P. E., Egholm, M., Berg, R. H., Buchardt, 0. and Stanley, C., Single base pair mutation analysis by PNA directed PCR clamping. Nucleic Acids Res. 21:5332-5336 (1993).
Pearson, R. B. and Kemp, B. E., Protein kinase phosphorylation site sequences and consensus specificity motifs: tabulations. Methods in Enzymol. 200:62-81 (1991).
Pope, A. J., Haupts, U., and Moore, K. J., Homogeneous fluorescence readouts for miniaturized high-throughput screening; theory and practice. Drug Discov. Today 4:350-362 (1999).
Sato, K., Tokesh, M., Odake, T., Kimura, H., Ooi, T., Nakao, M., and Kitamori, T., Integration of an immunosorbent assay system: analysis of secretory human immunoglobulin A on polystyrene beads in a microchip. Anal. Chem. 72:1144-7 (2000).
Sutherland, R. M., Dahne, C. Place, J. F. and Ringrose, A. S., Optical detection of antibody-antigen reactions at a glass-liquid interface. Clin, Chem., 30:1533-1538 (1984).
Travers, A. and Buckle, M. in DNA-Protein Interactions: A Practical Approach, (Oxford University Press, 2000).
Uhlmann, E. and Peyman, A., Antisense oligonucleotides: a new therapeutic principle. Chem. Rev. 90: 543-584 (1990).
Van Vunakis H., Radioimmunoassays: an overview. Methods Enzymol. 70(A):201-9 (1980).
Webb, M. R., A continuous spectrophotometric assay for inorganic phosphate and for measuring phosphate release kinetics in biological systems. Proc. Natl. Acad. Sci. 89:4884-4887 (1992).
Wunsch, E., Synthesis of naturally occurring polypeptides, problems of current research, Angew Chem. Int. Ed. 10:786-795 (1971).
Zhou, M., Diwu, Z., Pandhuk-Voloshina, N. and Haugland, R. P., A stable nonfluorescent derivative of resorufin for the fluorometric determination of trace hydrogen peroxide: applications in detecting the activity of phagocyte NADPH oxidase and other oxidases. Anal. Biochem. 253:162-168 (1997).
Bachem catalog, pp. 749-758 (2002).
New England Biolabs catalog, p. 162 (2000).
Pierce 2001-2002 Catalog, “Bifunctional cross-linking reagents”, pp. 294-331.
WEB page, “The Protein Kinase Resources” at http://pkr.sdsc.edu/html/disease.shtml.

The invention has been described with reference to preferred embodiments thereof, however, it will be appreciated that those skilled in the art, upon consideration of this disclosure, may make modifications and improvements within the spirit and scope of the invention.

1. A probe comprising at least one and preferably all of the following as operably linked components:
a) a first pair of nucleic acid sequences consisting of a first object sequence and a first complement sequence, said first object and first complement sequences each independently having about 3 to about 150 nucleotides, being substantially complementary to each other, and forming a first hybridized duplex;
b) at least one recognition element conjugated to at least one of said first object and first complement sequences, said recognition element specifically interacting with at least one target agent; and
c) optionally, at least one detectable label, said detectable label producing a characteristic signal whose level is a function of the amount of said first hybridized duplex,
wherein in the presence of said target agent, said interaction of said target agent with said recognition element alters the amount of said first hybridized duplex compared to that in the absence of said target agent, altering said characteristic signal.

2. The probe of claim 1 further comprising a second pair of competing nucleic acid sequences consisting of a second object sequence and a second complement sequence,
said second object and second complement sequences each independently having about 3 to about 150 nucleotides, being substantially complementary to each other, and forming a second hybridized duplex; and
said first and second object sequences being contained in an object sequence and having an overlapping region consisting of at least one nucleotide,
wherein in the presence of said target agent, said interaction of said target agent with said recognition element causes decrease in the amount of one of said first and second hybridized duplexes and increase in the amount of the other hybridized duplex compared to those in the absence of said target agent, altering said characteristic signal.

3. A probe according to claim 2, wherein said recognition element is conjugated to said first object sequence excluding said overlapping region or the first complement sequence.

4. A probe according to any of claims 1 and 2, wherein said object and complement sequences are selected from the group consisting of DNA, RNA, PNA and mixtures of DNA and RNA.

5. An affinity probe according to claim 1, wherein said recognition element is a probe ligand, and said target agent is a receptor agent that can specifically bind to said probe ligand.

6. An affinity probe according to claim 5, wherein the melting temperature of said first hybridized duplex decreases by at least 1° C. upon binding of said receptor agent to said probe ligand.

7. An affinity probe according to claim 5, wherein said probe ligand is covalently linked to said first object or first complement sequence by at least one coupling elements.

8. An affinity probe according to claim 7, wherein said coupling element is selected from the group consisting of chemical bonds, divalent atoms, divalent chemical moieties, and multivalent chemical moieties.

9. An affinity probe according to claim 5, wherein said probe ligand is selected from the group consisting of chemical ligands, biochemical ligands, antigens, antibodies, antibody fragments, enzymes, substrates of enzymes, substrates or inhibitors of enzymes, hormones, antibiotics, narcotics, toxins, polypeptides, proteins, protein fragments, targeting sequences, transit peptides, glycoproteins, lipids, phospholipids, polysaccaharides, carbohydrates, nucleic acids, and peptide nucleic acids.

10. A cleavage probe according to claim 1, wherein said recognition element is a probe substrate comprising a destabilizing agent and at least one cleavage site, and said target agent is a reaction-inducing agent that can specifically cleave said cleavage site, wherein a first end of said cleavage site is conjugated to said destabilizing agent and a second end of said cleavage site is covalently linked to said first object or first complement sequence by at least one coupling element.

11. A cleavage probe according to claim 10, wherein the melting temperature of said first hybridized duplex increases by at least 1° C. upon cleavage of said cleavage site by said reaction inducing agent.

12. A cleavage probe according to claim 10, wherein said coupling element is selected from the group consisting of chemical bonds, divalent atoms, divalent chemical moieties, and multivalent chemical moieties.

13. A cleavage probe according to claim 10, wherein said destabilizing agent is a protein or a protein complex covalently or non-covalently linked to the first end of said cleavage site by a linker.

14. A cleavage probe according to claim 13, wherein said destabilizing agent is a streptavidin or its derivative bound to a biotin, wherein the biotin is covalently linked to the first end of said cleavage site by a linker.

15. A cleavage probe according to claim 13, wherein said linker is selected from the group consisting of chemical bonds, divalent atoms, divalent chemical moieties, and multivalent chemical moieties.

16. A cleavage probe according to claim 10, wherein said cleavage site is specifically cleaved by a protease, an endonuclease, a lipase, or a glycosidase.

17. A cleavage probe according to claim 16, wherein said cleavage site is specifically cleaved by heparanase-1.

18. A cleavage probe according to claim 16, wherein said cleavage site is specifically cleaved by a mammalian or viral protease.

19. A cleavage probe according to claim 16, wherein said cleavage site is specifically cleaved by a protease associated with a human pathogen.

20. A cleavage probe according to claim 19, wherein the protease is expressed by a cytomegalovirus (CMV); herpes simplex virus (HSV); hepatitis virus; a plasmodium, human immunodeficiency virus (HIV), Kaposi's sarcoma-associated herpes virus (KSHV), yellow fever virus, flavivirus, or rhinovirus.

21. A cleavage probe according to claim 18, wherein the protease is a serine-type, cystein-type, or aspartate-type protease.

22. A cleavage probe according to claim 20, wherein the plasmodium is P falciparum and the protease is one of plasmepsin I and plasmepsin II.

23. A cleavage probe according to claim 20, wherein said cleavage site is specifically cleaved by a maturational protease of HSV.

24. A cleavage probe according to claim 20, wherein the hepatitis virus is type C.

25. A cleavage probe according to claim 19, wherein the human pathogen is yeast, bacterium, fungi, nematode, virus, or protozoa.

26. A cleavage probe according to claim 18, wherein said cleavage site is specifically cleaved by a mammalian protease associated with blood coagulation, apoptosis, Alzheimer's disease or the extracellular matrix.

27. A Type I coupling probe according to claim 1, wherein said recognition element is a probe substrate comprising at least one reaction site covalently linked to said first object or first complement sequence by at least one coupling element, and the target agent is a reaction-inducing agent that can specifically conjugate a destabilizing agent to said reaction site.

28. A Type I coupling probe according to claim 27, wherein the melting temperature of said first hybridized duplex decreases by at least 1° C. upon conjugation of said destabilizing agent by said reaction inducing agent.

29. A Type I coupling probe according to claim 27, wherein said coupling element is selected from the group consisting of chemical bonds, divalent atoms, divalent chemical moieties, and multivalent chemical moieties.

30. A Type I coupling probe according to claim 27, wherein said destabilizing agent is a protein or a protein complex having at least one reaction group that can be covalently linked to said reaction site in the presence of said reaction inducing agent.

31. A Type I coupling probe according to claim 30, wherein said destabilizing agent is a streptavidin bound to a modified biotin that has at least one said reaction group.

32. A Type I coupling probe according to claim 27, wherein said reaction site is specifically conjugated to said destabilizing agent by a ligase.

33. A Type I coupling probe according to claim 32, wherein said ligase is selected from the group consisting of polynucleotide ligases, aminoacyl tRNA ligases, and biotin protein ligases.

34. A Type II coupling probe according to claim 1, wherein said recognition element is a probe substrate comprising at least one reaction site covalently linked to said first object or first complement sequence by at least one coupling element, and the target agent is a reaction-inducing agent that can specifically convert said reaction site to a conjugation site to which a destabilizing agent can be conjugated.

35. A Type II coupling probe according to claim 34, wherein the melting temperature of said first hybridized duplex decreases by at least 1° C. upon conjugation of said destabilizing agent by said reaction inducing agent.

36. A Type II coupling probe according to claim 34, wherein said coupling element is selected from the group consisting of chemical bonds, divalent atoms, divalent chemical moieties, and multivalent chemical moieties.

37. A Type II coupling probe according to claim 34, wherein said destabilizing agent is a protein or a protein complex that can specifically bind to said conjugation site.

38. A Type II coupling probe according to claim 34, wherein said destabilizing agent is a protein or a protein complex having at least one reaction group that can be covalently linked to said conjugation site either spontaneously or in the presence of a coupling reagent.

39. A Type II coupling probe according to claim 38, wherein said destabilizing agent is a streptavidin or its derivative bound to a modified biotin that has at least one said reaction group.

40. A Type II coupling probe according to claim 34, wherein said reaction site is specifically converted by a transferase.

41. A Type II coupling probe according to claim 40, wherein said transferase is selected from the group consisting of kinases and acetyl-CoA transferases.

42. A Type II coupling probe according to claim 41, wherein said kinase is a protein kinase selected from the group consisting of c-AMP dependent protein kinase (PKA), casein kinase I (CKI), casein kinase II (CKII), glycogen synthase kinase 3 (GSK-3), cdc2 protein kinase, calmodulin-dependent protein kinase II (CaMK II), insulin receptor (INSR), mitogen-activated protein kinase (MAPK), cGMP-dependent protein kinase (cGPK), phosphorylase kinase (PhK), protein kinase C (PKC), p34 cdc2 protein kinase, meiosis-activated myelin basic protein kinase (p44 mpk), smooth muscle myosin light chain kinase, epidermal growth factor receptor kinase (EGF-RK), and protein tyrosine kinase pp60c-src (PTK).

43. A Type II coupling probe according to claim 34, wherein said reaction site is a site specific to a kinase, and said conjugation site includes a phosphate attached by the kinase.

44. A Type II coupling probe according to claim 43, wherein said destabilizing agent is an antibody or a receptor that can specifically bind to said conjugation site having the phosphate attached by the kinase.

45. A Type II coupling probe according to claim 43, wherein said phosphate is a thiophosphate and said destabilizing agent is a streptavidin or its derivative bound to a modified biotin that has at least one reaction group such as iodoacetyl or thiol group that can be covalently linked to said conjugation site in the presence of a coupling reagent.

46. A Type II coupling probe according to claim 43, wherein said destabilizing agent is a nanoparticle, a microparticle, a bead or a membrane made of synthetic polymers or the like containing dicataionic, tricataionic or polycataionic metal ions, that can specifically bind to said conjugation site having the phosphate group.

47. A Type II(-) coupling probe according to claim 1, wherein said recognition element is a probe substrate comprising at least one reaction site covalently linked to said first object or first complement sequence by at least one coupling element, and the target agent is a reaction-inducing agent that can specifically convert said reaction site that is a conjugation site to which a destabilizing agent can be conjugated, to a non-conjugatable site.

48. A Type II(-) coupling probe according to claim 47, wherein the melting temperature of said first hybridized duplex decreases by at least 1° C. upon conjugation of said destabilizing agent.

49. A Type II(-) coupling probe according to claim 47, wherein said coupling element is selected from the group consisting of chemical bonds, divalent atoms, divalent chemical moieties, and multivalent chemical moieties.

50. A Type II(-) coupling probe according to claim 47, wherein said destabilizing agent is a protein or a protein complex that can specifically bind to said reaction site.

51. A Type II(-) coupling probe according to claim 47, wherein said destabilizing agent is a protein or a protein complex having at least one reaction group that can be covalently linked to said reaction site either spontaneously or in the presence of a coupling reagent.

52. A Type II(-) coupling probe according to claim 51, wherein said destabilizing agent is a streptavidin or its derivative bound to a modified biotin that has at least one said reaction group.

53. A Type II(-) coupling probe according to claim 47, wherein said reaction site is a site having a phosphate group that can be specifically de-phosphorylated by a phosphatase.

54. A Type II(-) coupling probe according to claim 53, wherein said phosphatase is selected from phosphoprotein phosphatases such as protein tyrosine phosphatases, dual specificity phosphatases and protein Ser/Thr phosphatases; phospholipids phosphatases such as phosphatidylinositol-3,4-biphosphate 4-phosphatase, phosphatidylinositol-3,4,5-triphosphate 3-phosphatase, SH2 domain-containing inositol phosphatase (SHIP) and membrane-associated phospholipid phosphatase; and polynucleotide phosphatases such as polynucleotide 3′-phosphatase and polynucleotide 5′-phosphatase.

55. A Type II(-) coupling probe according to claim 53, wherein said destabilizing agent is an antibody or a receptor that can specifically bind to said reaction site having the phosphate group.

56. A Type II(-) coupling probe according to claim 53, wherein said phosphate is a thiophosphate and said destabilizing agent is a streptavidin or its derivative bound to a modified biotin that has at least one reaction group such as iodoacetyl or thiol group that can be covalently linked to said reaction site in the presence of a coupling reagent.

57. A Type II(-) coupling probe according to claim 53, wherein said destabilizing agent is a nanoparticle, a microparticle, a bead or a membrane made of synthetic polymers or the like containing dicataionic, tricataionic or polycataionic metal ions, that can specifically bind to said reaction site having the phosphate group.

58. An affinity probe according to claim 2, wherein said first hybridized duplex is preferentially formed compared to said second hybridized duplex in the absence of said target agent.

59. An affinity probe according to claim 58, wherein the melting temperature of said first hybridized duplex is at least 1° C. higher than the melting temperature of said second hybridized duplex in the absence of said target agent.

60. An affinity probe according to claim 58, wherein said second hybridized duplex is preferentially formed compared to said first hybridized duplex in the presence of an excess of said target agent.

61. An affinity probe according to claim 60, wherein the melting temperature of said first hybridized duplex is at least 1° C. lower than the melting temperature of said second hybridized duplex in the presence of an excess of said target agent.

62. A cleavage probe according to claim 2, wherein said second hybridized duplex is preferentially formed compared to said first hybridized duplex in the absence of said target agent.

63. A cleavage probe according to claim 62, wherein the melting temperature of said first hybridized duplex is at least 1° C. lower than the melting temperature of said second hybridized duplex in the absence of said target agent.

64. A cleavage probe according to claim 62, wherein said first hybridized duplex is preferentially formed compared to said second hybridized duplex in the presence of an excess of said target agent.

65. A cleavage probe according to claim 64, wherein the melting temperature of said first hybridized duplex is at least 1° C. higher than the melting temperature of said second hybridized duplex in the presence of an excess of said target agent.

66. A Type I coupling probe according to claim 2, wherein said first hybridized duplex is preferentially formed compared to said second hybridized duplex in the absence of said target agent.

67. A Type I coupling probe according to claim 66, wherein the melting temperature of said first hybridized duplex is at least 1° C. higher than the melting temperature of said second hybridized duplex in the absence of said target agent.

68. A Type I coupling probe according to claim 66, wherein said second hybridized duplex is preferentially formed compared to said first hybridized duplex in the presence of an excess of said target agent.

69. A Type I coupling probe according to claim 68, wherein the melting temperature of said first hybridized duplex is at least 1° C. lower than the melting temperature of said second hybridized duplex in the presence of an excess of said target agent.

70. A Type II coupling probe according to claim 2, wherein said first hybridized duplex is preferentially formed compared to said second hybridized duplex in the absence of said target agent.

71. A Type II coupling probe according to claim 70, wherein the melting temperature of said first hybridized duplex is at least 1° C. higher than the melting temperature of said second hybridized duplex in the absence of said target agent.

72. A Type II coupling probe according to claim 70, wherein said second hybridized duplex is preferentially formed compared to said first hybridized duplex in the presence of an excess of said target agent.

73. A Type II coupling probe according to claim 72, wherein the melting temperature of said first hybridized duplex is at least 1° C. lower than the melting temperature of said second hybridized duplex in the presence of an excess of said target agent.

74. A Type II(-) coupling probe according to claim 2, wherein said second hybridized duplex is preferentially formed compared to said first hybridized duplex in the absence of said target agent.

75. A Type II(-) coupling probe according to claim 74, wherein the melting temperature of said first hybridized duplex is at least 1° C. lower than the melting temperature of said second hybridized duplex in the absence of said target agent.

76. A Type II(-) coupling probe according to claim 74, wherein said first hybridized duplex is preferentially formed compared to said second hybridized duplex in the presence of an excess of said target agent.

77. A Type II(-) coupling probe according to claim 76, wherein the melting temperature of said first hybridized duplex is at least 1° C. higher than the melting temperature of said second hybridized duplex in the presence of an excess of said target agent.

78. A probe according to claim 1, wherein said detectable label is a non-interactive label selected from the group consisting of fluorescers, luminescers, radioisotopes, enzymes, antibodies, antigens, and electrochemical labels.

79. A probe according to claim 78, wherein said probe is a bimolecular probe consisting of a first molecule comprising said first object sequence and a second molecule comprising said first complement sequence, wherein said at least one detectable label is covalently linked to at least one of said first and second molecules.

80. A probe according to claim 79, wherein said first molecule is immobilized to a support and said at least one detectable label is covalently linked to said second molecule.

81. A probe according to claim 79, wherein said second molecule is immobilized to a support and said at least one detectable label is covalently linked to said first molecule.

82. A probe according to claim 1, wherein said detectable label is an intercalating dye that can preferentially bind to double-stranded nucleic acids.

83. A probe according to claim 82, wherein said probe comprises a first molecule comprising said first object sequence and a second molecule comprising said first complement sequence.

84. A probe according to claim 83, wherein said first or second molecule is immobilized to a support.

85. A probe according to claim 82, wherein said first object and first complement sequences are covalently linked by a loop moiety.

86. A probe according to claim 85, wherein said loop moiety connects any of 5′ or 3′ terminus of said first object sequence to any of 5′ or 3′ terminus of said first complement sequence.

87. A probe according to claim 85, wherein said loop moiety is a nucleic acid sequence having about 4 to about 100 nucleotides.

88. A probe according to claim 87, wherein said first object sequence, said loop moiety, and said first complement sequence are covalently linked in sequence in a 5′ to 3′ or 3′ to 5′ direction.

89. A probe according to claim 85 immobilized to a support.

90. A probe according to claim 1, wherein said at least one detectable label comprises an interactive label pair comprising a first label moiety conjugated to said first object sequence and a second label moiety conjugated to said first complement sequence, said first and second label moieties interacting when said first hybridized duplex is formed.

91. A probe according to claim 90, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

92. A probe according to claim 90, wherein said probe is a bimolecular probe consisting of a first molecule comprising said first object sequence including said first label moiety, and a second molecule comprising said first complement sequence including said second label moiety.

93. A probe according to claim 92, wherein said first or second molecule is immobilized to a support.

94. A probe according to claim 90, wherein said probe is a unimolecular probe, wherein said first object and first complement sequences are covalently linked by a loop moiety.

95. A probe according to claim 94, wherein said loop moiety connects any of 5′ or 3′ terminus of said first object sequence to any of 5′ or 3′ terminus of said first complement sequence.

96. A probe according to claim 94, wherein said loop moiety is a nucleic acid sequence having about 4 to about 100 nucleotides.

97. A probe according to claim 96, wherein said first object sequence, said loop moiety, and said first complement sequence are covalently linked in sequence in a 5′ to 3′ or 3′ to 5′ direction.

98. A probe according to claim 94 immobilized to a support.

99. A probe according to claim 1, wherein said probe is a unimolecular probe, wherein said first object and first complement sequences are covalently linked by a loop moiety and said at least one detectable label comprises an interactive label pair comprising first and second label moieties conjugated to different positions of said unimolecular probe, said first and second label moieties interacting when said first hybridized duplex is formed.

100. A probe according to claim 99, wherein said loop moiety connects any of 5′ or 3′ terminus of said first object sequence to any of 5′ or 3′ terminus of said first complement sequence.

101. A probe according to claim 99, wherein said loop moiety is a nucleic acid sequence having about 4 to about 100 nucleotides.

102. A probe according to claim 101, wherein said first object sequence, said loop moiety, and said first complement sequence are covalently linked in sequence in a 5′ to 3′ or 3′ to 5′ direction.

103. A probe according to claim 99, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

104. A probe according to claim 99, wherein at least one of said first and second label moieties is covalently linked to said loop moiety.

105. A probe according to claim 99 immobilized to a support.

106. A probe according to claim 1, wherein said probe further comprises a pair of nucleic acid arm sequences consisting of a 5′ arm sequence covalently linked to 5′ terminus of said first object sequence and a 3′ arm sequence covalently linked to 3′ terminus of said first object sequence, said pair of arm sequences forming a stem duplex having about 3 to about 35 complementary base pairs when said first object sequence is not hybridized to said first complement sequence; and wherein said at least one detectable label comprises an interactive label pair comprising a first label moiety conjugated to said 5′ arm sequence and a second label moiety conjugated to said 3′ arm sequence, said first and second label moieties interacting when said stem duplex is formed.

107. A probe according to claim 1, wherein said probe further comprises a pair of nucleic acid arm sequences consisting of a 5′ arm sequence covalently linked to 5′ terminus of said first complement sequence and a 3′ arm sequence covalently linked to 3′ terminus of said first complement sequence, said pair of arm sequences forming a stem duplex having about 3 to about 35 complementary base pairs when said first object sequence is not hybridized to said first complement sequence; and wherein said at least one detectable label comprises an interactive label pair comprising a first label moiety conjugated to said 5′ arm sequence and a second label moiety conjugated to said 3′ arm sequence, said first and second label moieties interacting when said stem duplex is formed.

108. A probe according to claim 1, wherein said probe further comprises two pairs of nucleic acid arm sequences, a first arm pair consisting of a first 5′ arm sequence covalently linked to 5′ terminus of said first object sequence and a first 3′ arm sequence covalently linked to 3′ terminus of said first object sequence and a second arm pair consisting of a second 5′ arm sequence covalently linked to 5′ terminus of said first complement sequence and a second 3′ arm sequence covalently linked to 3′ terminus of said first complement sequence, said first arm pair forming a first stem duplex having about 3 to about 35 complementary base pairs and said second arm pair forming a second stem duplex having about 3 to about 35 complementary base pairs when said first object sequence is not hybridized to said first complement sequence; and wherein said at least one detectable label comprises two interactive label pairs, a first label pair comprising a first label moiety conjugated to said first 5′ arm sequence and a second label moiety conjugated to said first 3′ arm sequence and a second label pair comprising a third label moiety conjugated to said second 5′ arm sequence and a fourth label moiety conjugated to said second 3′ arm sequence, said first label pair interacting when said first stem duplex is formed and said second label pair interacting when said second stem duplex is formed.

109. A probe according to any of claims 106-108, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

110. A probe according to any of claims 106-108, wherein said probe is a bimolecular probe consisting of a first molecule comprising said first object sequence and a second molecule comprising said first complement sequence.

111. A probe according to claim 110, wherein said first or second molecule is immobilized to a support.

112. A probe according to any of claims 106-108, wherein said probe is a unimolecular probe, wherein a first unit containing at least said first object sequence and a second unit containing at least said first complement sequence are covalently linked by a loop moiety.

113. A probe according to claim 112, wherein said loop moiety connects any of 5′ or 3′ terminus of said first object sequence to any of 5′ or 3′ terminus of said first complement sequence.

114. A probe according to claim 112, wherein said loop moiety is a nucleic acid sequence having about 4 to about 100 nucleotides.

115. A probe according to claim 114, wherein said first object sequence, said loop moiety, and said first complement sequence are covalently linked in sequence in a 5′ to 3′ or 3′ to 5′ direction.

116. A probe according to claim 112 immobilized to a support.

117. A probe according to claim 2, wherein said detectable label is a non-interactive label selected from the group consisting of fluorescers, luminescers, radioisotopes, enzymes, antibodies, antigens, and electrochemical labels.

118. A probe according to claim 117, wherein said probe is a trimolecular probe consisting of a first molecule comprising said object sequence, a second molecule comprising said first complement sequence, and a third molecule comprising said second complement sequence, wherein said at least one detectable label is covalently linked to at least one of said first, second, and third molecules.

119. A probe according to claim 118, wherein said at least one detectable label is covalently linked to at least one of said second and third molecules, wherein if both molecules are labeled, each molecule has a different label.

120. A probe according to claim 119, wherein said first molecule is immobilized to a support.

121. A probe according to claim 118, wherein one of said second or third molecule is immobilized to a support and said at least one detectable label is covalently linked to said first molecule.

122. A probe according to claim 117, wherein said probe is a bimolecular probe consisting of a first molecule comprising said object sequence and one of said first and second complement sequences, and a second molecule containing a remaining sequence, wherein the two sequences contained in said first molecule are covalently linked by a loop moiety and said at least one detectable label is covalently linked to at least one of said first and second molecules.

123. A probe according to claim 122, wherein said loop moiety connects any of 5′ or 3′ terminus of a first sequence to any of 5′ or 3′ terminus of a second sequence.

124. A probe according to claim 122, wherein said loop moiety is a nucleic acid sequence having about 4 to about 100 nucleotides.

125. A probe according to claim 124, wherein a first sequence, said loop moiety, and a second sequence are covalently linked in sequence in a 5′ to 3′ or 3′ to 5′ direction.

126. A probe according to claim 122, wherein said first molecule is immobilized to a support and said at least one detectable label is covalently linked to said second molecule.

127. A probe according to claim 122, wherein said second molecule is immobilized to a support and said at least one detectable label is covalently linked to said first molecule.

128. A probe according to claim 2, wherein said detectable label is an intercalating dye that can preferentially bind to double-stranded nucleic acids.

129. A probe according to claim 128, wherein said probe comprises a first molecule comprising said object sequence, a second molecule comprising said first complement sequence, and a third molecule comprising said second complement sequence.

130. A probe according to claim 129, wherein said second or third molecule is immobilized to a support.

131. A probe according to claim 2, wherein said at least one detectable label comprises an interactive label pair comprising a first label moiety conjugated to said first object sequence and a second label moiety conjugated to said first complement sequence, said first and second label moieties interacting when said first hybridized duplex is formed.

132. A probe according to claim 2, wherein said at least one detectable label comprises an interactive label pair comprising a first label moiety conjugated to said second object sequence and a second label moiety conjugated to said second complement sequence, said first and second label moieties interacting when said second hybridized duplex is formed.

133. A probe according to claim 2, wherein said at least one detectable label comprises two different interactive label pairs, a first label pair comprising a first label moiety conjugated to said first object sequence and a second label moiety conjugated to said first complement sequence and a second label pair comprising a third label moiety conjugated to said second object sequence and a fourth label moiety conjugated to said second complement sequence, said first and second label moieties interacting when said first hybridized duplex is formed and said third and fourth label moieties interacting when said second hybridized duplex is formed.

134. A probe according to claim 133, wherein said first and third label moieties are a same moiety.

135. A probe according to any of claims 131-133, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

136. A probe according to any of claims 131-133, wherein said probe is a trimolecular probe consisting of a first molecule comprising said object sequence, a second molecule comprising said first complement sequence, and a third molecule comprising said second complement sequence.

137. A probe according to claim 136, wherein one of said first, second, and third molecules is immobilized to a support.

138. A probe according to any of claims 131-133, wherein said probe is a bimolecular probe consisting of a first molecule containing two of said object sequence, said first complement sequence, and said second complement sequence, and a second molecule containing a remaining sequence, wherein said two sequences contained in said first molecule are covalently linked by a loop moiety.

139. A probe according to claim 138, wherein said loop moiety connects any of 5′ or 3′ terminus of a first sequence to any of 5′ or 3′ terminus of a second sequences.

140. A probe according to claim 138, wherein said loop moiety is a nucleic acid sequence having about 4 to about 100 nucleotides.

141. A probe according to claim 140, wherein a first sequence, said loop moiety, and a second sequence are covalently linked in sequence in a 5′ to 3′ or 3′ to 5′ direction.

142. A probe according to claim 138, wherein said first or second molecules is immobilized to a support.

143. A probe according to any of claims 131-133, wherein said probe is a unimolecular probe, wherein said object sequence, said first complement sequence, and said second complement sequence are covalently linked by at least one loop moiety.

144. A probe according to claim 143, wherein said loop moiety is a nucleic acid sequence having about 4 to about 100 nucleotides.

145. A probe according to claim 144, wherein said first complement sequence, a first loop moiety, said object sequence, a second loop moiety, and said second complement sequence are covalently linked in sequence in a 5′ to 3′ or 3′ to 5′ direction.

146. A probe according to claim 144, wherein said object sequence, a first loop moiety, said first complement sequence, a second loop moiety, and said second complement sequence are covalently linked in sequence in a 5′ to 3′ or 3′ to 5′ direction.

147. A probe according to claim 144, wherein said object sequence, a first loop moiety, said second complement sequence, a second loop moiety, and said first complement sequence are covalently linked in sequence in a 5′ to 3′ or 3′ to 5′ direction.

148. A probe according to claim 143 immobilized to a support.

149. A probe according to claim 2, wherein said probe is a unimolecular probe, wherein said object sequence, said first complement sequence, and said second complement sequence are covalently linked by at least one loop moiety, and said at least one detectable label comprises at least one interactive label pair, each label pair comprising first and second label moieties conjugated to different positions of said unimolecular probe, said first and second label moieties interacting when said first or second hybridized duplex is formed.

150. A probe according to claim 149, wherein at least one of said first and second label moieties is covalently linked to said loop moiety.

151. A probe according to claim 149, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

152. A probe according to claim 149 immobilized to a support.

153. A probe according to claim 2, wherein said probe is a bimolecular probe consisting of a first molecule containing two of said object sequence, said first complement sequence, and said second complement sequence, and a second molecule containing a remaining sequence, wherein said two sequences contained in said first molecule are covalently linked by a loop moiety, and said at least one detectable label comprises at least one interactive label pair, each label pair comprising first and second label moieties conjugated to different positions of said bimolecular probe, said first and second label moieties interacting when said first or second hybridized duplex is formed.

154. A probe according to claim 153, wherein at least one of said first and second label moieties is covalently linked to said loop moiety.

155. A probe according to claim 153, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

156. A probe according to claim 153, wherein said first or second molecule is immobilized to a support.

157. A probe according to claim 2, wherein said probe further comprises a pair of nucleic acid arm sequences consisting of a 5′ arm sequence covalently linked to 5′ terminus of said first complement sequence and a 3′ arm sequence covalently linked to 3′ terminus of said first complement sequence, said pair of arm sequences forming a stem duplex having about 3 to about 35 complementary base pairs when said first complement sequence is not hybridized to said first object sequence; and wherein said at least one detectable label comprises an interactive label pair comprising a first label moiety conjugated to said 5′ arm sequence and a second label moiety conjugated to said 3′ arm sequence, said first and second label moieties interacting when said stem duplex is formed.

158. A probe according to claim 2, wherein said probe further comprises a pair of nucleic acid arm sequences consisting of a 5′ arm sequence covalently linked to 5′ terminus of said second complement sequence and a 3′ arm sequence covalently linked to 3′ terminus of said second complement sequence, said pair of arm sequences forming a stem duplex having about 3 to about 35 complementary base pairs when said second complement sequence is not hybridized to said second object sequence; and wherein said at least one detectable label comprises an interactive label pair comprising a first label moiety conjugated to said 5′ arm sequence and a second label moiety conjugated to said 3′ arm sequence, said first and second label moieties interacting when said stem duplex is formed.

159. A probe according to claim 2, wherein said probe further comprises two pairs of nucleic acid armn sequences, a first arm pair consisting of a first 5′ arm sequence covalently linked to 5′ terminus of said first complement sequence and a first 3′ arm sequence covalently linked to 3′ terminus of said first complement sequence and a second arm pair consisting of a second 5′ arm sequence covalently linked to 5′ terminus of said second complement sequence and a second 3′ arm sequence covalently linked to 3′ terminus of said second complement sequence, said first arm pair forming a first stem duplex having about 3 to about 35 complementary base pairs when said first complement sequence is not hybridized to said first object sequence and said second arm pair forming a second stem duplex having about 3 to about 35 complementary base pairs when said second complement sequence is not hybridized to said second object sequence; and wherein said at least one detectable label comprises two interactive label pairs, a first label pair comprising a first label moiety conjugated to said first 5′ arm sequence and a second label moiety conjugated to said first 3′ arm sequence and a second label pair consisting of a third label moiety conjugated to said second 5′ arm sequence and a fourth label moiety conjugated to said second 3′ arm sequence, said first label pair interacting when said first stem duplex is formed and said second label pair interacting when said second stem duplex is formed.

160. A probe according to any of claims 157-159, wherein said interactive label pair is a fluorescer and a quencher, wherein the interaction between said interactive label pair causes change in the fluorescence of said probe, said change in the fluorescence being selected from the group consisting of intensity decrease, lifetime decrease, polarization change, and wavelength change.

161. A probe according to any of claims 157-159, wherein said probe is a trimolecular probe consisting of a first molecule comprising said object sequence, a second molecule comprising said first complement sequence, and a third molecule comprising said second complement sequence.

162. A probe according to claim 161, wherein one of said first, second, and third molecules is immobilized to a support.

163. A probe according to any of claims 157-159, wherein said probe is a bimolecular probe consisting of a first molecule containing two of said object sequence, said first complement sequence, and said second complement sequence, and a second molecule containing a remaining sequence, wherein said two sequences contained in said first molecule are covalently linked by a loop moiety.

164. A probe according to claim 163, wherein one of said first and second molecules is immobilized to a support.

165. A probe according to any of claims 157-159, wherein said probe is a unimolecular probe, wherein a first molecule comprising said object sequence, a second molecule comprising said first complement sequence, and a third molecule comprising said second complement sequence are covalently linked by at least one loop moiety.

166. A probe according to claim 165 immobilized to a support.

167. An assay for detecting in a sample the presence or absence of at least one target receptor agent that can selectively bind to a probe ligand under the conditions including a detection temperature, said assay comprising:
a) contacting said sample with a probe according to claim 5; and
b) ascertaining any change in the level of said characteristic signal at said detection temperature compared to that in the absence of said target receptor agent.

168. An assay for detecting in a sample the presence or absence of at least one target ligand under the conditions including a detection temperature, said assay comprising:
a) contacting said sample with a probe according to claim 5 in the presence of a receptor agent that can bind to both said probe and target ligands; and
b) ascertaining any change in the level of said characteristic signal at said detection temperature compared to that in the absence of said target ligand.

169. An assay for detecting in a sample the presence or absence of at least one target reaction inducing agent that can specifically cleave a cleavage site under the conditions including a detection temperature, said assay comprising:
a) contacting said sample with a probe according to claim 10; and
b) ascertaining any change in the level of said characteristic signal at said detection temperature compared to that in the absence of said target reaction inducing agent.

170. An assay for detecting in a sample the presence or absence of at least one target reaction inducing agent that can specifically induce a covalent coupling of a reaction site under the conditions including a detection temperature, said assay comprising:
a) contacting said sample with a probe according to claim 27; and
b) ascertaining any change in the level of said characteristic signal at said detection temperature compared to that in the absence of said target reaction inducing agent.

171. An assay for detecting in a sample the presence or absence of at least one target reaction inducing agent that can specifically convert a reaction site to a conjugation site under the conditions including a detection temperature, said assay comprising:
a) contacting said sample with a probe according to claim 34; and
b) ascertaining any change in the level of said characteristic signal at said detection temperature compared to that in the absence of said target reaction inducing agent.

172. An assay for detecting in a sample the presence or absence of at least one target reaction inducing agent that can specifically convert a reaction site to a non-conjugatable site under the conditions including a detection temperature, said assay comprising:
a) contacting said sample with a probe according to claim 47; and
b) ascertaining any change in the level of said characteristic signal at said detection temperature compared to that in the absence of said target reaction inducing agent.

173. An assay for detecting an inhibitor or enhancer for binding of a receptor agent to a probe ligand under the conditions including a detection temperature, said assay comprising:
a) contacting a target compound with a probe according to claim 5 in the presence of said receptor agent; and
b) ascertaining any change in the level of said characteristic signal at said detection temperature compared to that in the absence of said target candidate compound.

174. An assay for detecting an inhibitor or enhancer for a reaction inducing agent that can specifically cleave a cleavage site under the conditions including a detection temperature, said assay comprising:
a) contacting a target compound with a probe according to claim 10 in the presence of said reaction inducing agent; and
b) ascertaining any change in the level of said characteristic signal at said detection temperature compared to that in the absence of said target compound.

175. An assay for detecting an inhibitor or enhancer for at least one reaction inducing agent that can specifically induce a covalent coupling of a reaction site under the conditions including a detection temperature, said assay comprising:
a) contacting a target compound with a probe according to claim 27 in the presence of said reaction inducing agent and a destabilizing agent; and
b) ascertaining any change in the level of said characteristic signal at said detection temperature compared to that in the absence of said target compound.

176. An assay for detecting an inhibitor or enhancer for at least one reaction inducing agent that can specifically convert a reaction site to a conjugation site under the conditions including a detection temperature, said assay comprising:
a) contacting a target compound with a probe according to claim 34 in the presence of said reaction inducing agent and a destabilizing agent; and
b) ascertaining any change in the level of said characteristic signal at said detection temperature compared to that in the absence of said target compound.

177. An assay for detecting an inhibitor or enhancer for at least one reaction inducing agent that can specifically convert a reaction site to a non-conjugatable site under the conditions including a detection temperature, said assay comprising:
a) contacting a target compound with a probe according to claim 47 in the presence of said reaction inducing agent and a destabilizing agent; and
b) ascertaining any change in the level of said characteristic signal at said detection temperature compared to that in the absence of said target compound.

178. An assay according to any of claims 167-177, wherein the step of ascertaining comprises measuring the level of said characteristic signal.

179. An assay according to claim 178, wherein the step of ascertaining comprises measuring the level of said characteristic signal as a function of time.

180. An assay according to any of claims 167, 168, and 173 further comprising a step of contacting a target-less control with a probe according to claim 5, and a step of measuring the level of said characteristic signal at said detection temperature, wherein the step of ascertaining includes calculating a difference between the level of said characteristic signal in said control and that in said sample.

181. An assay according to any of claims 169 and 174 further comprising a step of contacting a target-less control with a probe according to claim 10, and a step of measuring the level of said characteristic signal at said detection temperature, wherein the step of ascertaining includes calculating a difference between the level of said characteristic signal in said control and that in said sample.

182. An assay according to any of claims 170 and 175 further comprising a step of contacting a target-less control with a probe according to claim 27, and a step of measuring the level of said characteristic signal at said detection temperature, wherein the step of ascertaining includes calculating a difference between the level of said characteristic signal in said control and that in said sample.

183. An assay according to any of claims 171 and 176 further comprising a step of contacting a target-less control with a probe according to claim 34, and a step of measuring the level of said characteristic signal at said detection temperature, wherein the step of ascertaining includes calculating a difference between the level of said characteristic signal in said control and that in said sample.

184. An assay according to any of claims 172 and 177 further comprising a step of contacting a target-less control with a probe according to claim 47, and a step of measuring the level of said characteristic signal at said detection temperature, wherein the step of ascertaining includes calculating a difference between the level of said characteristic signal in said control and that in said sample.

185. An assay according to any of claims 167, 168, and 173, wherein said detection temperature is at least 5° C. lower than the melting temperature of said first hybridized duplex in the absence of said receptor agent.

186. An assay according to any of claims 169 and 174, wherein said detection temperature is at least 5° C. higher than the melting temperature of said first hybridized duplex in the absence of said reaction inducing agent.

187. An assay according to any of claims 170, 171, 175, and 176, wherein said detection temperature is at least 5° C. lower than the melting temperature of said first hybridized duplex in the absence of said reaction inducing agent.

188. An assay according to any of claims 172 and 177, wherein said detection temperature is at least 5° C. higher than the melting temperature of said first hybridized duplex in the absence of said reaction inducing agent.

189. An assay according to any of claims 167-177, wherein said probe is a bimolecular or trimolecular probe having at least one non-interactive label.

190. An assay according to claim 189, wherein said probe is not an immobilized probe and the step of ascertaining includes a step of separating hybridized and unhybridized species.

191. An assay according to claim 189, wherein said probe is an immobilized probe and the step of ascertaining includes a step of washing out unbound species.

192. An assay according to any of claims 167-177, wherein said detectable label is an intercalating dye.

193. An assay according to claim 192, wherein said probe is not an immobilized probe and the step of ascertaining includes a step of separating hybridized and unhybridized species.

194. An assay according to claim 192, wherein said probe is an immobilized probe and the step of ascertaining includes a step of washing out unbound species.

195. An assay according to any of claims 167-177, wherein said probe comprises at least one interactive label pair.

196. An assay according to 195, wherein said interactive label pair is a pair of a fluorescer and a quencher or a pair of a luminescer and a quencher.

197. An assay according to claim 195, wherein said probe is a unimolecular probe.

198. An assay according to claim 195, wherein said probe is a bimolecular probe.

199. An assay according to claim 195, wherein said probe is a trimolecular probe.

200. An assay according to claim 195, wherein said probe is an immobilized probe.

201. An assay according to any of claims 167-172, wherein said probe is a unimolecular probe comprising at least one interactive label pair consisting of a fluorescer and a quencher or a luminescer and a quencher.

202. An assay according to claim 201, wherein said sample is a cell or tissue, and the step of contacting includes introducing said probe inside or outside the cell or tissue.

203. An assay according to claim 173, wherein said probe is a unimolecular probe comprising at least one interactive label pair consisting of a fluorescer and a quencher or a luminescer and a quencher.

204. An assay according to claim 203, wherein said sample is a cell or tissue, and the step of contacting includes introducing said probe and said target compound inside or outside the cell or tissue.

205. An assay according to claim 204, wherein said receptor agent is a receptor present inside or outside the cell or tissue.

206. An assay according to claim 204, wherein said receptor agent is a receptor protein recombinantly expressed by introducing a vector encoding the receptor protein inside the cell or tissue.

207. An assay according to any of claims 174-177, wherein said probe is a unimolecular probe comprising at least one interactive label pair consisting of a fluorescer and a quencher or a luminescer and a quencher.

208. An assay according to claim 207, wherein said sample is a cell or tissue, and the step of contacting includes introducing said probe and said target compound inside or outside the cell or tissue.

209. An assay according to claim 208, wherein said reaction inducing agent is an enzyme present inside or outside the cell or tissue.

210. An assay according to claim 209, wherein said reaction inducing agent is an enzyme recombinantly expressed by introducing a vector encoding the enzyme inside the cell or tissue.

211. An assay for detecting in a sample the presence or absence of at least one target receptor agent that can selectively bind to a probe ligand under the conditions including a detection temperature, said assay comprising:
c) contacting said sample with a probe according to claim 58; and
d) ascertaining any change in the level of said characteristic signal at said detection temperature compared to that in the absence of said target receptor agent.

212. An assay for detecting in a sample the presence or absence of at least one target ligand under the conditions including a detection temperature, said assay comprising:
c) contacting said sample with a probe according to claim 58 in the presence of a receptor agent that can bind to both said probe and target ligands; and
d) ascertaining any change in the level of said characteristic signal at said detection temperature compared to that in the absence of said target ligand.

213. An assay for detecting in a sample the presence or absence of at least one target reaction inducing agent that can specifically cleave a cleavage site under the conditions including a detection temperature, said assay comprising:
c) contacting said sample with a probe according to claim 62; and
d) ascertaining any change in the level of said characteristic signal at said detection temperature compared to that in the absence of said target reaction inducing agent.

214. An assay for detecting in a sample the presence or absence of at least one target reaction inducing agent that can specifically induce a covalent coupling of a reaction site under the conditions including a detection temperature, said assay comprising:
c) contacting said sample with a probe according to claim 66; and
d) ascertaining any change in the level of said characteristic signal at said detection temperature compared to that in the absence of said target reaction inducing agent.

215. An assay for detecting in a sample the presence or absence of at least one target reaction inducing agent that can specifically convert a reaction site to a conjugation site under the conditions including a detection temperature, said assay comprising:
c) contacting said sample with a probe according to claim 70; and
d) ascertaining any change in the level of said characteristic signal at said detection temperature compared to that in the absence of said target reaction inducing agent.

216. An assay for detecting in a sample the presence or absence of at least one target reaction inducing agent that can specifically convert a reaction site to a non-conjugatable site under the conditions including a detection temperature, said assay comprising:
c) contacting said sample with a probe according to claim 73; and
d) ascertaining any change in the level of said characteristic signal at said detection temperature compared to that in the absence of said target reaction inducing agent.

217. An assay for detecting an inhibitor or enhancer for binding of a receptor agent to a probe ligand under the conditions including a detection temperature, said assay comprising:
c) contacting a target compound with a probe according to claim 58 in the presence of said receptor agent; and
d) ascertaining any change in the level of said characteristic signal at said detection temperature compared to that in the absence of said target candidate compound.

218. An assay for detecting an inhibitor or enhancer for a reaction inducing agent that can specifically cleave a cleavage site under the conditions including a detection temperature, said assay comprising:
c) contacting a target compound with a probe according to claim 62 in the presence of said reaction inducing agent; and
d) ascertaining any change in the level of said characteristic signal at said detection temperature compared to that in the absence of said target compound.

219. An assay for detecting an inhibitor or enhancer for at least one reaction inducing agent that can specifically induce a covalent coupling of a reaction site under the conditions including a detection temperature, said assay comprising:
c) contacting a target compound with a probe according to claim 66 in the presence of said reaction inducing agent and a destabilizing agent; and
d) ascertaining any change in the level of said characteristic signal at said detection temperature compared to that in the absence of said target compound.

220. An assay for detecting an inhibitor or enhancer for at least one reaction inducing agent that can specifically convert a reaction site to a conjugation site under the conditions including a detection temperature, said assay comprising:
c) contacting a target compound with a probe according to claim 70 in the presence of said reaction inducing agent and a destabilizing agent; and
d) ascertaining any change in the level of said characteristic signal at said detection temperature compared to that in the absence of said target compound.

221. An assay for detecting an inhibitor or enhancer for at least one reaction inducing agent that can specifically convert a reaction site to a non-conjugatable site under the conditions including a detection temperature, said assay comprising:
c) contacting a target compound with a probe according to claim 73 in the presence of said reaction inducing agent and a destabilizing agent; and
d) ascertaining any change in the level of said characteristic signal at said detection temperature compared to that in the absence of said target compound.

222. A kit comprising at least one probe according to any of claims 1 and 2 and instructions for performing an assay for detecting at least one target agent or target ligand, or for detecting inhibitors or enhancers that inhibit or enhance interaction of the target agent with said recognition element.

223. A kit according to claim 211 further including one or more reagents selected from the group consisting of salts, buffers, nuclease inhibitors, substrates for enzyme or enzyme-coupled labels, receptor agents, reaction-inducing agents, vectors encoding receptor proteins or enzymes that act as target agents, and coupling reagents.

224. A kit according to claim 211 further including one or more components suitable for performing an in vivo or in situ assay, wherein said component is a component necessary for introducing said probe into a cell selected from permeabilizing agents and liposome precursors.

225. A kit according to claim 211, wherein said probe is an immobilized probe.

226. A kit according to claim 214 including at least one additional immobilized probe according to this invention having a different target agent and immobilized to the same support at a predetermined location.

227. A kit according to claim 214 including at least one additional immobilized probe according to this invention having a same target agent and immobilized to the same support at a different predetermined location.

228. A target detection system comprising at least one probe according to any of claims 1 and 2.


SEQUENCE LISTING (167 sequences)

SEQ ID NO: 1 - protein, 7 aa, Human immunodeficiency virus type 1
  MISC_FEATURE (1)..(7): HIV-1 protease cleavage site
SQNYPIV

SEQ ID NO: 2 - protein, 7 aa, Human immunodeficiency virus type 1
  MISC_FEATURE (1)..(7): HIV-1 protease cleavage site
ARVLAEA

SEQ ID NO: 3 - protein, 7 aa, Human immunodeficiency virus type 1
  MISC_FEATURE (1)..(7): HIV-1 protease cleavage site
ATIMMQR

SEQ ID NO: 4 - protein, 7 aa, Human immunodeficiency virus type 1
  MISC_FEATURE (1)..(7): HIV-1 protease cleavage site
RQANFLG

SEQ ID NO: 5 - protein, 7 aa, Human immunodeficiency virus type 1
  MISC_FEATURE (1)..(7): HIV-1 protease cleavage site
PGNFLQS

SEQ ID NO: 6 - protein, 5 aa, Human immunodeficiency virus type 1
  MISC_FEATURE (1)..(5): HIV-1 protease cleavage site
SVPQI

SEQ ID NO: 7 - protein, 7 aa, Human immunodeficiency virus type 1
  MISC_FEATURE (1)..(7): HIV-1 protease cleavage site
SFNFPQI

SEQ ID NO: 8 - protein, 7 aa, Human immunodeficiency virus type 1
  MISC_FEATURE (1)..(7): HIV-1 protease cleavage site
TLNFPIS

SEQ ID NO: 9 - protein, 7 aa, Human immunodeficiency virus type 1
  MISC_FEATURE (1)..(7): HIV-1 protease cleavage site
AETFYVD

SEQ ID NO: 10 - protein, 7 aa, Human immunodeficiency virus type 1
  MISC_FEATURE (1)..(7): HIV-1 protease cleavage site
RKVLFLD

SEQ ID NO: 11 - protein, 10 aa, Hepatitis C virus
  MISC_FEATURE (1)..(10): HCV NS3 protease cleavage site
DEMEECATHL

SEQ ID NO: 12 - protein, 10 aa, Hepatitis C virus
  MISC_FEATURE (1)..(10): HCV NS3 protease cleavage site
CSTPCSGSWL

SEQ ID NO: 13 - protein, 10 aa, Hepatitis C virus
  MISC_FEATURE (1)..(10): HCV NS3 protease cleavage site
EDVVCCSMSY

SEQ ID NO: 14 - protein, 10 aa, Hepatitis C virus
  MISC_FEATURE (1)..(10): HCV NS3 protease cleavage site
EDVVPCSMSY

SEQ ID NO: 15 - protein, 10 aa, Hepatitis C virus
  MISC_FEATURE (1)..(10): HCV NS3 protease cleavage site
DEMEECASHL

SEQ ID NO: 16 - protein, 10 aa, Hepatitis C virus
  MISC_FEATURE (1)..(10): HCV NS3 protease cleavage site
MQTTCPCGAQ

SEQ ID NO: 17 - protein, 10 aa, Hepatitis C virus
  MISC_FEATURE (1)..(10): HCV NS3 protease cleavage site
DDIVCCSMSY

SEQ ID NO: 18 - protein, 5 aa, human herpesvirus 1
  MISC_FEATURE (1)..(5): HSV-1 protease cleavage site
LVLAS

SEQ ID NO: 19 - protein, 6 aa, Human herpesvirus 1
  MISC_FEATURE (1)..(6): HSV-1 protease cleavage site
LVLASS

SEQ ID NO: 20 - protein, 12 aa, Human T-cell lymphotropic virus type 1
  MISC_FEATURE (1)..(12): HTLV-1 protease cleavage site
SAPQVLPVMHPN

SEQ ID NO: 21 - protein, 12 aa, Human T-cell lymphotropic virus type 1
  MISC_FEATURE (1)..(12): HTLV-1 protease cleavage site
SKTKVLVVQPKN

SEQ ID NO: 22 - protein, 5 aa, human cytomegalovirus
  MISC_FEATURE (1)..(5): HCMV Assemblin cleavage site
VVNAS

SEQ ID NO: 23 - protein, 6 aa, human cytomegalovirus
  MISC_FEATURE (1)..(6): HCMV Assemblin cleavage site
VVNASS

SEQ ID NO: 24 - protein, 17 aa, mammalian
  MISC_FEATURE (1)..(17): Alpha-secretase cleavage site
VHHQKLVFFAEDVGSNK

SEQ ID NO: 25 - protein, 10 aa, mammalian
  MISC_FEATURE (1)..(10): Beta-secretase cleavage site
SEVKMDAEFR

SEQ ID NO: 26 - protein, 10 aa, mammalian
  MISC_FEATURE (1)..(10): Beta-secretase cleavage site
SEVNLDAEFR

SEQ ID NO: 27 - protein, 15 aa, mammalian
  MISC_FEATURE (1)..(15): Gamma-secretase cleavage site
RRGGVVIATVIVGER

SEQ ID NO: 28 - protein, 8 aa, mammalian
  MISC_FEATURE (1)..(8): Plasmin cleavage site
PRFKIIGG

SEQ ID NO: 29 - protein, 4 aa, mammalian
  MISC_FEATURE (1)..(4): Matrix metallo proteinase-1 cleavage site
PLGL

SEQ ID NO: 30 - protein, 4 aa, mammalian
  MISC_FEATURE (1)..(4): Matrix metallo proteinase-1 cleavage site
PLGI

SEQ ID NO: 31 - protein, 8 aa, mammalian
  MISC_FEATURE (1)..(8): Matrix metallo proteinase-3 cleavage site
IPENFFGV

SEQ ID NO: 32 - protein, 8 aa, mammalian
  MISC_FEATURE (1)..(8): Matrix metallo proteinase-3 cleavage site
GPEGLRVG

SEQ ID NO: 33 - protein, 8 aa, mammalian
  MISC_FEATURE (1)..(8): Matrix metallo proteinase-3 cleavage site
RVGFYESD

SEQ ID NO: 34 - protein, 8 aa, mammalian
  MISC_FEATURE (1)..(8): Matrix metallo proteinase-3 cleavage site
LLSALVET

SEQ ID NO: 35 - protein, 8 aa, mammalian
  MISC_FEATURE (1)..(8): Matrix metallo proteinase-3 cleavage site
EAIPMSIP

SEQ ID NO: 36 - protein, 8 aa, mammalian
  MISC_FEATURE (1)..(8): Matrix metallo proteinase-3 cleavage site
RAIHIQAE

SEQ ID NO: 37 - protein, 8 aa, mammalian
  MISC_FEATURE (1)..(8): Matrix metallo proteinase-3 cleavage site
PFSPLVAT

SEQ ID NO: 38 - protein, 8 aa, mammalian
  MISC_FEATURE (1)..(8): Aggrecanase cleavage site
TEGEARGS

SEQ ID NO: 39 - protein, 5 aa, mammalian
  MISC_FEATURE (1)..(5): Caspase-1 cleavage site
YVHDA

SEQ ID NO: 40 - protein, 5 aa, mammalian
  MISC_FEATURE (1)..(5): Caspase-1 cleavage site
DGPDG

SEQ ID NO: 41 - protein, 5 aa, mammalian
  MISC_FEATURE (1)..(5): Caspase-1 cleavage site
DEVDG

SEQ ID NO: 42 - protein, 5 aa, mammalian
  MISC_FEATURE (1)..(5): Caspase-3 cleavage site
DEVDN

SEQ ID NO: 43 - protein, 5 aa, mammalian
  MISC_FEATURE (1)..(5): Caspase-3 cleavage site
DMQDN

SEQ ID NO: 44 - protein, 4 aa, mammalian
  MISC_FEATURE (1)..(4): Caspase-3 cleavage site
DXXD

SEQ ID NO: 45 - protein, 5 aa, mammalian
  MISC_FEATURE (1)..(5): Caspase-3 cleavage site
DEPDS

SEQ ID NO: 46 - protein, 5 aa, mammalian
  MISC_FEATURE (1)..(5): Caspase-3 cleavage site
DEADG

SEQ ID NO: 47 - protein, 5 aa, mammalian
  MISC_FEATURE (1)..(5): Caspase-3 cleavage site
DETDS

SEQ ID NO: 48 - protein, 5 aa, mammalian
  MISC_FEATURE (1)..(5): Caspase-3 cleavage site
DAVDT

SEQ ID NO: 49 - protein, 4 aa, mammalian
  MISC_FEATURE (1)..(4): Caspase-3 cleavage site
ESMD

SEQ ID NO: 50 - protein, 5 aa, mammalian
  MISC_FEATURE (1)..(5): Caspase-4 cleavage site
LEVDX

SEQ ID NO: 51 - protein, 5 aa, mammalian
  MISC_FEATURE (1)..(5): Caspase-4 cleavage site
WEHDX

SEQ ID NO: 52 - protein, 5 aa, mammalian
  MISC_FEATURE (1)..(5): Caspase-4 cleavage site
LEHDX

SEQ ID NO: 53 - protein, 5 aa, mammalian
  MISC_FEATURE (1)..(5): Caspase-6 cleavage site
VEIDN

SEQ ID NO: 54 - protein, 5 aa, mammalian
  MISC_FEATURE (1)..(5): Caspase-8 cleavage site
IETDX

SEQ ID NO: 55 - protein, 5 aa, mammalian
  MISC_FEATURE (1)..(5): Caspase-8 cleavage site
LETDX

SEQ ID NO: 56 - protein, 4 aa, mammalian
  MISC_FEATURE (1)..(4): Caspase-10 cleavage site
IEAD

SEQ ID NO: 57 - protein, 9 aa, mammalian
  MISC_FEATURE (1)..(9): Angiotensin converting enzyme cleavage site
RPPGFSPFR

SEQ ID NO: 58 - protein, 10 aa, mammalian
  MISC_FEATURE (1)..(10): Angiotensin converting enzyme cleavage site
DDVYIHPFHL

SEQ ID NO: 59 - protein, 7 aa, mammalian
  MISC_FEATURE (1)..(7): Platelet glydoprotein V thrombin cleavage site
PGPRGPP

SEQ ID NO: 60 - protein, 8 aa, mammalian
  MISC_FEATURE (1)..(8): Factor IX cleavage site
NLTRIVGG

SEQ ID NO: 61 - protein, 14 aa, mammalian
  MISC_FEATURE (1)..(14): Thrombin cleavage site
TVELQGVVPRGVNL

SEQ ID NO: 62 - protein, 14 aa, mammalian
  MISC_FEATURE (1)..(14): Thrombin cleavage site
TVELQGLVPRGVNL

SEQ ID NO: 63 - protein, 14 aa, mammalian
  MISC_FEATURE (1)..(14): Thrombin cleavage site
DFLAEGGGVRGPRV

SEQ ID NO: 64 - protein, 14 aa, mammalian
  MISC_FEATURE (1)..(14): Thrombin cleavage site
KATNATLDPRSFLL

SEQ ID NO: 65 - protein, 8 aa, Plasmodium
  MISC_FEATURE (1)..(8): Plasmepsin I cleavage site
ERMFLSFP

SEQ ID NO: 66 - protein, 8 aa, Plasmodium
  MISC_FEATURE (1)..(8): Plasmepsin I cleavage site
FPHFDLSH

SEQ ID NO: 67 - protein, 8 aa, Plasmodium
  MISC_FEATURE (1)..(8): Plasmepsin I cleavage site
VNFKLLSH

SEQ ID NO: 68 - protein, 8 aa, Plasmodium
  MISC_FEATURE (1)..(8): Plasmepsin I cleavage site
LGRLLVVY

SEQ ID NO: 69 - protein, 8 aa, Plasmodium
  MISC_FEATURE (1)..(8): Plasmepsin I cleavage site
TQRFFESF

SEQ ID NO: 70 - protein, 8 aa, Plasmodium
  MISC_FEATURE (1)..(8): Plasmepsin I cleavage site
VQAAYQKV

SEQ ID NO: 71 - protein, 8 aa, Plasmodium
  MISC_FEATURE (1)..(8): Plasmepsin II cleavage site
LLVTLAAH

SEQ ID NO: 72 - protein, 8 aa, Plasmodium
  MISC_FEATURE (1)..(8): Plasmepsin II cleavage site
STVLTSKY

SEQ ID NO: 73 - protein, 8 aa, Plasmodium
  MISC_FEATURE (1)..(8): Plasmepsin II cleavage site
GRLLVVYP

SEQ ID NO: 74 - protein, 8 aa, Plasmodium
  MISC_FEATURE (1)..(8): Falcilysin cleavage site
TTKTYFPH

SEQ ID NO: 75 - protein, 8 aa, Plasmodium
  MISC_FEATURE (1)..(8): Falcilysin cleavage site
AHVDDMPN

SEQ ID NO: 76 - protein, 8 aa, Plasmodium
  MISC_FEATURE (1)..(8): Falcilysin cleavage site
HAHKLRVD

SEQ ID NO: 77 - protein, 8 aa, Plasmodium
  MISC_FEATURE (1)..(8): Falcilysin cleavage site
WTQRFFES

SEQ ID NO: 78 - protein, 8 aa, Plasmodium
  MISC_FEATURE (1)..(8): Falcilysin cleavage site
AFSDGLAH

SEQ ID NO: 79 - protein, 8 aa, Plasmodium
  MISC_FEATURE (1)..(8): Falcilysin cleavage site
LAHLDNLK

SEQ ID NO: 80 - protein, 8 aa, Plasmodium
  MISC_FEATURE (1)..(8): Falcilysin cleavage site
AYQKVVAG

SEQ ID NO: 81 - protein, 8 aa, Schistoma
  MISC_FEATURE (1)..(8): Cathepsin cleavage site
AEALERMF

SEQ ID NO: 82 - protein, 8 aa, Schistoma
  MISC_FEATURE (1)..(8): Cathepsin cleavage site
FLSFPTTK

SEQ ID NO: 83 - protein, 8 aa, Schistoma
  MISC_FEATURE (1)..(8): Cathepsin cleavage site
TPEEKASV

SEQ ID NO: 84 - protein, 8 aa, Schistoma
  MISC_FEATURE (1)..(8): Cathepsin cleavage site
VTALWEKV

SEQ ID NO: 85 - protein, 8 aa, Schistoma
  MISC_FEATURE (1)..(8): Cathepsin cleavage site
LGRLLLVV

SEQ ID NO: 86 - protein, 11 aa, mammalian
  MISC_FEATURE (1)..(11): cAMP-dependent protein Kinase phosphorylation site
YLRRASLAQLT

SEQ ID NO: 87 - protein, 8 aa, mammalian
  MISC_FEATURE (1)..(8): cAMP-dependent protein Kinase phosphorylation site
FRRLSIST

SEQ ID NO: 88 - protein, 11 aa, mammalian
  MISC_FEATURE (1)..(11): cAMP-dependent protein Kinase phosphorylation site
AGARRKASGPP

SEQ ID NO: 89 - protein, 8 aa, mammalian
  MISC_FEATURE (1)..(8): cAMP-dependent protein Kinase phosphorylation site
GRGLSLSR

SEQ ID NO: 90 - protein, 11 aa, mammalian
  MISC_FEATURE (1)..(11): Casein Kinase I phosphorylation site; Ser (location4) phosphorylated
RTLSVSSLPGL

SEQ ID NO: 91 - protein, 10 aa, mammalian
  MISC_FEATURE (1)..(10): Casein Kinase I phosphorylation site; Ser (location4 and 6) phosphorylated
DIGSESTEDQ

SEQ ID NO: 92 - protein, 10 aa, mammalian
  MISC_FEATURE (1)..(10): Casein Kinase II phosphorylation site
ADSESEDEED

SEQ ID NO: 93 - protein, 11 aa, mammalian
  MISC_FEATURE (1)..(11): Casein Kinase II phosphorylation site
LESEEEGVPST

SEQ ID NO: 94 - protein, 11 aa, mammalian
  MISC_FEATURE (1)..(11): Casein Kinase II phosphorylation site
EDNSEDEISNL

SEQ ID NO: 95 - protein, 9 aa, mammalian
  MISC_FEATURE (1)..(9): Glycogen Synthase Kinase 3 phosphorylation site Ser (location9) phosphorylated
SVPPSPSLS

SEQ ID NO: 96 - protein, 9 aa, mammalian
  MISC_FEATURE (1)..(9): Glycogen Synthase Kinase 3 phosphorylation site Ser (location 5 and 9) phosphorylated
SVPPSPSLS

SEQ ID NO: 97 - protein, 7 aa, mammalian
  MISC_FEATURE (1)..(7): Cdc2 protein Kinase phosphorylation site
PAKTPVK

SEQ ID NO: 98 - protein, 10 aa, mammalian
  MISC_FEATURE (1)..(10): Cdc2 protein Kinase phosphorylation site
HSTPPKKKRK

SEQ ID NO: 99 - protein, 11 aa, mammalian
  MISC_FEATURE (1)..(11): Calmodulin-dependent protein Kinase II phosphorylation site
NYLRRRLSDSN

SEQ ID NO: 100 - protein, 10 aa, mammalian
  MISC_FEATURE (1)..(10): Calmodulin-dependent protein Kinase II phosphorylation site
KMARVFSVLR

SEQ ID NO: 101 - protein, 13 aa, mammalian
  MISC_FEATURE (1)..(13): Insulin receptor phosphorylation site
RRLIEDAEYAARG

SEQ ID NO: 102 - protein, 4 aa, mammalian
  MISC_FEATURE (1)..(4): Mitogen-activated protein Kinase (Extracellular Signal-regulated Kinase) phosphorylation site
PLSP

SEQ ID NO: 103 - protein, 4 aa, mammalian
  MISC_FEATURE (1)..(4): Mitogen-activated protein Kinase (Extracellular Signal-regulated Kinase) phosphorylation site
PSSP

SEQ ID NO: 104 - protein, 4 aa, mammalian
  MISC_FEATURE (1)..(4): Mitogen-activated protein Kinase (Extracellular Signal-regulated Kinase) phosphorylation site
VLSP

SEQ ID NO: 105 - protein, 21 aa, mammalian
  MISC_FEATURE (1)..(21): Mitogen-activated protein Kinase (Extracellular Signal-regulated Kinase) phosphorylation site
KRELVEPLTPSGEAPNQALLR

SEQ ID NO: 106 - protein, 11 aa, mammalian
  MISC_FEATURE (1)..(11): cGMP-dependent protein Kinase phosphorylation site
GKKRKRSRKES

SEQ ID NO: 107 - protein, 8 aa, mammalian
  MISC_FEATURE (1)..(8): cGMP-dependent protein Kinase phosphorylation site
FRRLSIST

SEQ ID NO: 108 - protein, 7 aa, mammalian
  MISC_FEATURE (1)..(7): cGMP-dependent protein Kinase phosphorylation site
RKRSRAE

SEQ ID NO: 109 - protein, 12 aa, mammalian
  MISC_FEATURE (1)..(12): Phosphorylase Kinase phosphorylation site
DQEKRKQISVRG

SEQ ID NO: 110 - protein, 10 aa, mammalian
  MISC_FEATURE (1)..(10): Phosphorylase Kinase phosphorylation site
PLSRTLSVSS

SEQ ID NO: 111 - protein, 9 aa, mammalian
  MISC_FEATURE (1)..(9): Protein Kinase C phosphorylation site
HEGTHSTKR

SEQ ID NO: 112 - protein, 10 aa, mammalian
  MISC_FEATURE (1)..(10): Protein Kinase C phosphorylation site
PLSRTLSVSS

SEQ ID NO: 113 - protein, 11 aa, mammalian
  MISC_FEATURE (1)..(11): Protein Kinase C phosphorylation site
QKRPSQRSKYL

SEQ ID NO: 114 - protein, 12 aa, mammalian
  MISC_FEATURE (1)..(12): Protein Kinase C phosphorylation site
PLSRTLSVAAKK

SEQ ID NO: 115 - protein, 7 aa, mammalian
  MISC_FEATURE (1)..(7): Protein Kinase C phosphorylation site
LKFSKKF

SEQ ID NO: 116 - protein, 8 aa, mammalian
  MISC_FEATURE (1)..(8): Protein Kinase C phosphorylation site
RKRTLRRL

SEQ ID NO: 117 - protein, 21 aa, mammalian
  MISC_FEATURE (1)..(21): p34 cdc2 protein Kinase phosphorylation site
AKAQHATPPKKKRKVEDPKDF

SEQ ID NO: 118 - protein, 9 aa, mammalian
  MISC_FEATURE (1)..(9): Meiosis-activated myelin basic protein Kinase phosphorylation site
APRTPGGRR

SEQ ID NO: 119 - protein, 11 aa, mammalian
  MISC_FEATURE (1)..(11): Smooth Muscle Myosin Light Chain Kinase phosphorylation site
KKRARTSNVFA

SEQ ID NO: 120 - protein, 11 aa, mammalian
  MISC_FEATURE (1)..(11): Epidermal Growth Factor Receptor Kinasephosphorylation site
RENAEYLRVAP

SEQ ID NO: 121 - protein, 10 aa, mammalian
  MISC_FEATURE (1)..(10): Epidermal Growth Factor Receptor Kinase phosphorylation site
AEPDYGALYE

SEQ ID NO: 122 - protein, 5 aa, mammalian
  MISC_FEATURE (1)..(5): Protein Tyrosine Kinase pp60c-src phosphorylation site
IYGEF

SEQ ID NO: 123 - DNA, 52 bp, Artificial
  Synthetic Sequence
atggaagtatatggaagtatatggaagtattcgtggggttttgcagtcgtag

SEQ ID NO: 124 - DNA, 14 bp, Artificial
  Synthetic Sequence
gactgcaaaacccc

SEQ ID NO: 125 - DNA, 52 bp, Artificial
  Synthetic Sequence
atggaagtatatggaagtatatggaagtattcgtggggntttgcagtcgtag

SEQ ID NO: 126 - DNA, 16 bp, Artificial
  Synthetic Sequence
gactgcaaaaccccac

SEQ ID NO: 127 - DNA, 36 bp, Artificial
  Synthetic Sequence
gcagcctaggaaacaccaaagatgatatttggctgc

SEQ ID NO: 128 - DNA, 38 bp, Artificial
  Synthetic Sequence
gcagctctaggaaacaccaaagatgatatttgagctgc

SEQ ID NO: 129 - DNA, 30 bp, Artificial
  Synthetic Sequence
aaatatcatctttggtgtttcctaggctgc

SEQ ID NO: 130 - DNA, 14 bp, Artificial
  Synthetic Sequence
gactgcaaaacccc

SEQ ID NO: 131 - DNA, 12 bp, Artificial
  Synthetic Sequence
ctacgactgcaa

SEQ ID NO: 132 - DNA, 52 bp, Artificial; identical to SEQ ID NO: 123
  Synthetic Sequence

SEQ ID NO: 133 - DNA, 14 bp, Artificial
  Synthetic Sequence
gactgcaaaacccc

SEQ ID NO: 134 - DNA, 16 bp, Artificial
  Synthetic Sequence
gactgcaaaaccccac

SEQ ID NO: 135 - DNA, 52 bp, Artificial; identical to SEQ ID NO: 123
  Synthetic Sequence

SEQ ID NO: 136 - DNA, 16 bp, Artificial
  Synthetic Sequence
gactgcaaaaccccac

SEQ ID NO: 137 - DNA, 52 bp, Artificial; identical to SEQ ID NO: 125
  Synthetic Sequence

SEQ ID NO: 138 - DNA, 14 bp, Artificial
  Synthetic sequence
gcaggactacctgc

SEQ ID NO: 139 - DNA, 16 bp, Artificial
  Synthetic sequence
gcaggactttacctgc

SEQ ID NO: 140 - DNA, 18 bp, Artificial
  Synthetic sequence
gcaggactcattacctgc

SEQ ID NO: 141 - DNA, 25 bp, Artificial
  Synthetic sequence
gcaggatactcattaccatacctgc

SEQ ID NO: 142 - DNA, 35 bp, Artificial
  Synthetic sequence
gcaggatactcattagcgacgaacaccatacctgc

SEQ ID NO: 143 - DNA, 45 bp, Artificial
  Synthetic sequence
gcaggatacttagaccaacacattagcgacgaacaccatacctgc

SEQ ID NO: 144 - DNA, 25 bp, Artificial
  Synthetic sequence
cgaccatcctcattaccataggtcg

SEQ ID NO: 145 - DNA, 25 bp, Artificial
  Synthetic sequence
gcagcatcctcattaccctagctgc

SEQ ID NO: 146 - DNA, 25 bp, Artificial
  Synthetic sequence
cgacgatcctcattaccatacgtcg

SEQ ID NO: 147 - DNA, 25 bp, Artificial
  Synthetic sequence
ggaggataatcattaccatacctcc

SEQ ID NO: 148 - DNA, 25 bp, Artificial
  Synthetic sequence
ccaccatactcattaccctaggtgg

SEQ ID NO: 149 - DNA, 23 bp, Artificial
  Synthetic sequence
gcagatactcattaccatactgc

SEQ ID NO: 150 - DNA, 25 bp, Artificial
  Synthetic sequence
gcaggatactgcttaccatacctgc

SEQ ID NO: 151 - DNA, 25 bp, Artificial
  Synthetic sequence
gcaggactctcattacactgcctgc

SEQ ID NO: 152 - DNA, 25 bp, Artificial
  Synthetic sequence
agcgcatcctcattaccctagcgct

SEQ ID NO: 153 - DNA, 25 bp, Artificial
  Synthetic sequence
gcgcaatcctcattaccctatgcgc

SEQ ID NO: 154 - DNA, 25 bp, Artificial
  Synthetic sequence
gcagcatcctcattaccctagctgc

SEQ ID NO: 155 - DNA, 25 bp, Artificial
  Synthetic sequence
gcagcatcctcattaccctagctgc

SEQ ID NO: 156 - DNA, 25 bp, Artificial
  Synthetic sequence
gcagcatcctcattaccctagctgc

SEQ ID NO: 157 - DNA, 25 bp, Artificial
  Synthetic sequence
gcagctaggagtaatgggatgctgc

SEQ ID NO: 158 - DNA, 15 bp, Artificial
  Synthetic sequence
atcccattactccta

SEQ ID NO: 159 - DNA, 13 bp, Artificial
  Synthetic sequence
atcccattactcc

SEQ ID NO: 160 - DNA, 15 bp, Artificial
  Synthetic sequence
tagggtaatgaggat

SEQ ID NO: 161 - DNA, 25 bp, Artificial
  Synthetic sequence
gcagcatcctcattaccctagctgc

SEQ ID NO: 162 - DNA, 25 bp, Artificial
  Synthetic sequence
gcagcatcctcattaccctagctgc

SEQ ID NO: 163 - protein, 7 aa, Artificial
  Synthetic sequence
KRTLRRC

SEQ ID NO: 164 - protein, 6 aa, mammalian
  MISC_FEATURE (1)..(6): Protein Kinase C phosphorylation site
KRTLRR

SEQ ID NO: 165 - DNA, 25 bp, Artificial
  Synthetic sequence
gcagcatcctcattaccctagctgc

SEQ ID NO: 166 - protein, 7 aa, Artificial
  Synthetic sequence
KRTLRRC

SEQ ID NO: 167 - DNA, 25 bp, Artificial
  Synthetic sequence
gcagcatcctcattaccctagctgc